SEQUENCE LISTING (136 sequences)

SEQ ID NO: 1 - protein, 662 aa, Unknown
  Pseudomonas specie
MAPVCWQFAGSSSATEARPRMIRIFLLLIVCLTTLPWTALVMAAAQAPVLQTRTEAPLQVGAMEYLLGEPAADFVDIAAADRQWQILTKPNLGKQREGAWLRFTLHNHGDDAERWYLLLKWPVLDRVAVRLHYPQSERWGTAMLAGDALALSVRPLADHHFVYPLELPPGEPVVVYMHVQAREILALPLELIDEKQLIEGKLRDVTLVSLFFGGILVIVLYNCSLLIFTRDRSYFLYVLYLLSAVFYVLTITGFGQLYLWPEIPELSARFYGLSAALCFFTPMLFALRFLGIRRYGGWVWTVSITLTCYWGLVTLTILLAPLLARYLFMEHVALLHCVLTMAVTLNLWARGNPSARLFSIAWSTLLAFTVINLLALNGTLPLNAWTLNGQLIGMFTEFVLLSMALAERINVERNRRIAAQQLALQASESLAEERALHLQAKQAALDFQLHTNELLEARVFERTRALEEVRQGLEAANAELMRLSTTDALTQLANRRRFDRLLDEEIRRARRSGSPLSVLLADIDHFKRVNDSYGHPFGDECLRQVAAVLAAHCQRAGDVAARYGGEEFVVLLPGLDAEQAFALAESIRCTIANLQLQHGDQPVALTISLGVATLTPALVAPAELLAAADSALYQAKNGGRNQVVLAPDCVTEYAKAISPQPV

SEQ ID NO: 2 - protein, 526 aa, Unknown
  Pseudomonas specie
MRLNQPVSGREVVVGETANILSTTDLKGTITYANPDFVAISGFSEDELLGQPHNIVRHPDMPEAAFADLWQTLQANRSWMGMVKNRCKNGDHYWVSAFATPVSRDGKVKEYQSVRTRPQSEQIRAAEALYAQLRDGRGMPRLRGTVDLRERVALLAAAGAAGGVIVGTVLATSPIAATLAGLALGGTVAVAVAMTLLPLSRLARQARQVGDNPVSQLIYTGRKDEIGQIEFAMKMLETEAGAMVGRIADSSRQLSDHARDLLGAMHGSTQSAARQHQETDLIATAIQQMSASVQEVALNAQRTAEVAARADSEAARGREVVGRTGSSIIRLASDIQQAAEVIHQLENHSQEISRVLEVIHGIAEQTNLLALNAAIEAARAGEQGRGFAVVADEVRQLASRTSLATTDIQRMIGSLQTGAREAVDVMQRSREQAEHSVSHADQAEHSLSGINSRVNEICAMSSQIAAAVDQQSSASEEISQSIVSIRGSSDDHVASGLHSQRNATGVALLADGMLELVQQFWARRRG

SEQ ID NO: 3 - protein, 80 aa, Unknown
  Pseudomonas specie
MTNRCSVLLFFPRIGFQLFKQIDQASAQLAELPRLERTAGTLQYLDGFFQGIAQGLFAQHWFFFVHSYAGWVPLPSVRTG

SEQ ID NO: 4 - protein, 534 aa, Unknown
  Pseudomonas specie
MFTRPAMRMNLPVTGRDVSFSETANILSTTDLNGDITYVNPDFIKISGFEESELIGQHHNIVRHPDMPTEAFADLWGSVRGGKSWMGMVKNRCKNGDHYWVSAFVTPISRNGRVMEYQSVRTKPQPAQIAAAEQLYAQLRDKRAPAALRRKPLSARSRVALFAALGAAPCVVAGGLLGGAGFAIALATAAVAALGAGGLAYLALAPLQQLAEQARQIGDNPVGQLIYAGRRDEYGQIAFAMKMLETEAGAMVGRIGDASRQLSQHAHELLGAMESSTQSASRQQSETDQVATAINQMAASVQEVANNAQRTAEAASRADSETATGTEVVNRTGAAIGQLAQDIQQAGDVIHELEAHSNDITKVLDVIRGIAEQTNLLALNAAIEAARAGEQGRGFAVVADEVRSLASRTQQSTQEINSMIGALQGGARQAVEVMQRSREQAMQSVEHAAQAARSLQGINSRVNEISEMSQQIAAAVEQQSAVSENINQNIVSIRGGSDHHVANGLRSRHSASGVAELANSMEVLVQQFWTRRRS

SEQ ID NO: 5 - protein, 30 aa, Unknown
  Pseudomonas specie
MRLKSYAFGRFLQPAMNGHILYKKDKMYNL

SEQ ID NO: 6 - protein, 75 aa, Unknown
  Pseudomonas specie
MHSFLVVFGFPGIPCESARNLSLAGARLGNSPELWTRLSDPLVSAPTARTGSAGSGCVRLAVFYCSQVVHLVFLV

SEQ ID NO: 7 - protein, 183 aa, Unknown
  Pseudomonas specie
MKGKSLAWIFTAALAGTGLSGVVQAQEKFVTIGTGGQTGVYYVAGQSICRFVNRNAENIKCNAPASGGGVANVNGLRSGEFNFGIMQSDHQYKAMEGVAPFEKEGKMDDIRAVFSLQSEVFTVLARRSANIKGLDDLKGKWDPRVPSSNSPYSESYYNSLAVVLQRRDWENPGVTQLNRLAAH

SEQ ID NO: 8 - protein, 202 aa, Unknown
  Bradyrhizobium species
MNRRNLLLILATTTALSALALSAARARDLPKVATGFVANILCSETFVSGQQPDRIFAETTAAMPGAGLITWALDYHVDRARKDTTVTFLGLSRSHAVYREGFGCYLDHGDAVADLSVPPPDAKPLPALLPDIAGPAPVAPVNPQLAAALDRAFAEPEQPPLRATKAVVIVKDGHVVAERYAVGYGIDTPILGFSATKSVISA

SEQ ID NO: 9 - protein, 119 aa, Unknown
  Bradyrhizobium species
MIVEEDIEIAVAEPLIDQPPQSVFGDPELGRERTDRDVVVTVDALIAHQPHQRIDQLLIVCVLCRRRLSRNEHDGCSFRCSRKQVGAVDAHHRVANRRHDTGQNGGIHRVGDLSSRPTG

SEQ ID NO: 10 - protein, 138 aa, Unknown
  Bradyrhizobium species
MLATSVVAPPGNSICNTAFMPPYNAEAPTMKILTRRRRSTRSADHEEMVWVTAIVMKPSASRLIAMMEIRGLPAGAIHMQLTKLNVATAAQTSPCHKRNLKASSNSARVNSQNTTTLPVDSAINGACPIETGDVAPIE

SEQ ID NO: 11 - protein, 190 aa, Unknown
  Bradyrhizobium species
MIPSLKPGARLMRVDGPVIETGRLILRQWRSDDVAPNTAMLSDPGTARFIAADGKPITSELAGWRNAAVMSGHWALHGFGMFAVEEKSSGKYIGRVGPWSPPGWPGFEVGWGIAREYRGKGYAVEAARASIDWVFATFETDEIIHCIDLVNEPSQAVARRLGARKDREVDLFGHAADIWVTSRASWAVQN

SEQ ID NO: 12 - protein, 305 aa, Unknown
  Bradyrhizobium species
MSWQPSNDPVLGDPKSCDALELIIVPRTRDLGDGFEVRRALPHGKRQMVGPFIFFDHFGPVQFMAGKGMDVRPHPHIGLATVTYLFDGSIMHRDSEGNVQEIQPGAMNLMTAGRGIAHSERSPDVQRREGQKMLGLQSWIALPAGSEEIAPSFQHYAAESLPTVNDGGFTARIIAGSGFGVKSPVTMVSPWFYTEVTAVAGTSVPLDADHEERAIYVVEGEVEIAGERYESPRLLIFRPGDRITVKAVKPTRMMFLGGDALEGPRHIWWNFVSSSKERIEQAKQDWKTGRFAHVPQEHEFIPLPE

SEQ ID NO: 13 - protein, 342 aa, Unknown
  Bradyrhizobium species
MFPRNTSSFRCRSELTLAYRYAAARASPRARLPESKTQMNAMLASDLPLPRIGRGKVRDIYAVGDDRVLLLTTDRISAFDVVMAETIPMKGAVLTQISAWWFRQLKGVVPHHMISADADEIIKLVPALKDHRADIVGRAMLCRRTTVFPVECVIRGYISGSAWKEYAASGTLAGEKLAAGLVESEKLEPAIFSPATKAETGHDENITVAGVREILGAEVAQTLERMARTVYDFGEKISRRQGIIIADTKFEFGRDGDGRVILIDEVMTPDSSRFWAVDVYKPGQPQPSFDKQPLRDYLDTERRAGRWNGDAPPPPLPASVVDATSKRYLEAYRRVTGNALAI

SEQ ID NO: 14 - protein, 269 aa, Unknown
  Bradyrhizobium species
MTETDNLLVERDGPVTIVSINRPHCRNAVDGATARKLYDAFLAFDADEAASVAVFTGTGGYFCAGADLKAVASGDPNKKREIGGHDSIAPMGPSRLRLSKPVIAAVEGFAVAGGMELALWADMRVVAEDATFGIFCRRFGVPLIDLGTIRLPRLIGHSQAIDLILTGRPVGGPEALRMGLANRLVPKGEARAHAIALAKDIARFPQTCLRADRLSTLRQWDLEEEEAIANEMRGGLKVIASGETLSGATRFASGIGRHGAFGGDVVGNG

SEQ ID NO: 15 - protein, 725 aa, Unknown
  Bradyrhizobium species
MKPGIRPALSDIIGGSAASVLTVTFGLSYALLIFTGPLTPYLSYGVAATFIASAVLGAVVALGSSFPFAIAAPESSTAAMTGILASSLVEHMTTRDPSASLLAPVLITLGLSAIITGIVLCGFGVTRMGRAIRYVPYPVVGGFLGATGCLIVLGAIRVITGQRLQLATLDRFANILTLSELAAACAMALVLYLTWHRSRTAFGLPAILVGGIIVAHIAFRLAGITSTEAQASGWTFQPPPAVNIMLPWSAAEISHYPWYALPDLIGNLIAVIFVTASSTLFNTTGVEVASHREANLERELNVTGLANILSGTFGGFTGCISVSRSMLNLNSGGTSRLSGLTGAAISTIMLAAAPALLGYMPKFVLGGLLLYLGADQLHKWIVQSRRRLSFIEYLSLLAIIIIIVQWGFIAGTLIGVVIGCTTFALSASRIDSIKFGFDGSEYRSSLDRARDDQAVLSAHGGKIQGLNLQSYLFFGSANRLYERVKALLARHPECRFLIFDFKLVTGIDSSAAYSFAQIKRTAHDVGVKLVLVHLPAAAEKALRSSEFLSSEVNIVPELDHALEWCENEIILQHKGLEREEASLRDWFTEILGSERDAMELIHRCQRLEVEAGEIIVSAGDAADSMHFILDGRVGIMISNGEGGSTRVRSLGRYTTIGEMGLVSRSPRSATIQAEVASTLYVLTVEQFEAIKQDDPALGQKLLTYFVSVMAERLTFANRMIAVLRR

SEQ ID NO: 16 - protein, 498 aa, Unknown
  Bradyrhizobium species
MNPPVAARASQSSTPSLRDRLKDSSLLREQCYIDGAWTGKASRPVTNPVNGIELAKIPVMSTAETIQAVEAAGRAFPAWAKLTAKARSNILRKWFDLIIANREDLALILTSEQGKPLAEALGEVDIGGAYVEFFAEEARRVYGETIPTQRADARLLAIKQPIGVCGAITPWNFPCSMITRKVSPALAAGCTVVLKPANETPLTALALVALAEKAGVPKGVFNILTGNSSAIGKVLCEHPAVRFVGFTGSTEVGKILYQQAAVGVKKLGLELGGNAPFVVFDDADIDAAVEGAIVSKYRNMGQTCVCANRIYAQDKIYDQFVEKLSNKVAAMKVGDGTEAGVTQGPLINMDAIDKVEKHIADAVKGGAKIITGGKRHALGGSFFEPTVLANVKPDALVAHEETFGPLAPVFRFKDEAEVIAMCNNSPFGLASYFYSRDLGRVWRVAEALESGMVGVNTGLITTEVAPFGGVKESGLGREGSHHGMEEYVEIKYVMMAGV

SEQ ID NO: 17 - protein, 106 aa, Unknown
  Bradyrhizobium species
MISTWSEWKRYPRAGRGENIEAPVSPGLYEVRYAGTGALHSFAAVDNVAQALAQLPVGSKSWFLRRDPAKLPDLEYRTCATSSRADAKAAAERMIGRRETYMNGAA

SEQ ID NO: 18 - protein, 397 aa, Unknown
  Bradyrhizobium species
MQSKAQIDQILRQKCEAKEIPGVVAMAATGNEVIYQGAFGKRDLSKDDPMTTDSVFWIASMTKAITTAAGMQLVEQGKLSLDEPIGKVLPDLASPQVLKGFDAKGEPKLRPAKKPITLRHLMTHTAGFCYHLWNGDMANYLEKTGTPDITSCKNAALKTPIMSDPGTRWEYGTNIDFVGKAIEAVSGKRLDAYLRDHMLTPLGMTDTSFKLGDAQRKRLVGMHARGADGTLASIPFELEQDPEFHMGGGGLYGTAGDYIKFTQMILNKGKGNGNQVLKPETVAMMGQNHVGELSMTKLTSAVAWATNDVDLYPGIEKKWGLSFMINTAKTPEGRSPGSLAWAGLANTYFWIDPARNVSGVILMQLLPFVDQKCLEAYAGFERGIYAGLDAGSGQKAA

SEQ ID NO: 19 - protein, 583 aa, Unknown
  Bradyrhizobium species
MHVIRVMSNSRAPSHGEADLTDIAGSYVCGTADAPLLGETIGASLDLAVQRWGNREALVSPSHDVRWIWREFAERVDTLAAGFLALGLERGARIGVWSLNRPEWTLTQFAAAKAGIILVTINPAYRLSELEFALAKVGCAAIVTATAFKTSNYMEMLNTLLPELAKSVPGELQAARLPQLRAVIQIGGPASPGTIAFEDVTRMGGARHRDTLAELAKTLQFDDAVNIQFTSGTTGSPKGVTLTHHNILNNGYFVGRAMRLTEQDRICIPVPLYHCFGMVMGNLAAVTLGATMVYPGEGFDPLATLQTIEKEKCTTLYGVPTMFIAQLDHPEFSRFDLKSLRTGIMAGAPCPVEVMKRVNNEMNMSEVTIAYGMTETSPVSFQSSTDDPLERRVSTVGRIHPHVEVKVVDLEGRVVPRGERGELCTRGYSVMLGYWDEAEKTRDVLDANGWMHTGDIAIIDAAGYCNIVGRIKDMVIRGGENLYPREIEEFLYRHPKIQDVQIFGVADDRYGEELCAWIRTRPGETLTAEEVRGFCQGQIAHNKIPRYVEFVDEFPMTVTGKIQKFLMRDAVEAKLGLKAAKTA

SEQ ID NO: 20 - protein, 84 aa, Unknown
  Bradyrhizobium species
MIMNAGPAASFHSRRQYGERRRDKLTRRANYFRLSEIVSSPKIKNISLFPKRETGHICCHPVPLRGASAVVTNEGRVAVDAMRA

SEQ ID NO: 21 - protein, 70 aa, Unknown
  Bradyrhizobium species
MAQRGRPPGPERVPFPLRAEKAILEAVKRCAAADLRSVNAEIEILLREALARRGVRLKPANEDGEDSSDK

SEQ ID NO: 22 - protein, 297 aa, Unknown
  Bradyrhizobium species
MLALGLAGIVFLCCLSWIAFDPNNSSLVRAWIALSPVIIAALLAIGVLKGFVVLQPNESLVCLLFGSYVGTEHRPGFWWVNPFNSKHKVSRRLETLESGPLKVNDAVGNPIDIGAVIVWRVEDAAKASLEVASYASYVKSQSETALRRMVSVHPYDQVETEEAERQGDRPASAAISAVSLRDGGDAVIEALLGELRARMEPIGVAVLEARISHLAYSSEIASAMLRKQAASAVVAARRMIVRGAVSIVEDALNELDRKKLADALDPERKAAMISNLLVVLVGDREVSPVINTGTLYS

SEQ ID NO: 23 - protein, 316 aa, Unknown
  Bradyrhizobium species
MLVQTDEGFLARHGMTKGGMALIDEARATAIYADMGPATEMSGGSAANTIVGIANLGARAAYVGKVRDDQIGRLYTHDIRAAGVAFETKAASGGPATGCSYILVTPDGERTMNTYLGAAQELMPSDIDEAQIAASSIIYLEGYLWDPKSAKQAFVKAATIAHGAGRQVALTLSDSFCVDRYRDEFLDLMRKGTVDLVFANEAELHSLYQTSDFDIALKQLRADTKLGVVTRSEKGCVVASTDGVVAVPAFPIQKLVDTTGAGDLFAAGFLFGLVRNAGHEAAGRLGGLAAAEVIQHIGARPQVSLKELAQQNGLPV

SEQ ID NO: 24 - protein, 437 aa, Unknown
  Bradyrhizobium species
MFSHIRPIFQPYRNAATTASLAFGGARDQSRSTTAIGGKRTSLSQVSKSDQAEHCTSPNSGYRLGQARLSEVFWMGGEVVSDFLAGGGEMGALTRGFDWSKTSLGPPETWPQSLRVTVRLVLTSRHPMFIWWGPELIQFYNDAYRETMGPERHPSALGARGRECWDEIWDIIGPQIEYVMAGKGATWHEDQLVPVTRHGRREDVWWTYSFGPIDLEGKVGGVLVVCKDVTSEHMSKEALRLINEELKHRVKNTLTVLGAVATQTFRDVSSKADLEKYHGRLAAFGRAHDLLTAANWAAAPLQDVISTALIPYRTGEGRFVTSGPPLVVKSRQGLALSLAIHELATNALKYGALTVPSGHVSITWTSEDEVGQPKFCFEWREVGGPPASEPAGAGFGSRLISRVLKDDFGGSVEVSYGPTGLICRLTAPLENLATSLE

SEQ ID NO: 25 - protein, 518 aa, Unknown
  Bradyrhizobium species
MIRSLLTVSTGTLASRLLGFVRDSMIAALLGAGPVADAFLAAFQMVNVVRRLLTEGGLNASLVPAWLRVRDTDGAAAAAAFAGRVFGTITAALFAGTLLLALLMPLVLMVLAPGFSGGETLQLAVDDARLMLPYLAFAGPVTVMMALLNAQGRFALTAFSPLLFNVALIAVLFVLLASNANAVRAAQILAATVGVAGLLQLAILVLRRGSNIATPLRVSFDKEIRGFLAKAVPGMIASSTPQLLMVAGAIVASSSPSSVSWLYFANRLVELPLGIVGIAMGTVLIPEMTRALRGEDRTAVAHAESRGLELAVGLALPATLGLILLSEPVVRFLFEHGAFTATDTAATAHALMWLVLGLPANVLVKALSPAFFAREDTMTPLLAMLKGTAFALVLAVLLGHWFGASGVAAAIALGAWSTAFTLIRRGAETFGFSIDAAARRRLPGILAAALAMGAALWLAVRWLTPVLATSSHGLVIVLPILIIAGIAIYGLFLRLFGITGWREAIKAIRQNRAADLRD

SEQ ID NO: 26 - protein, 350 aa, Unknown
  Bradyrhizobium species
MAMVERVFSGVQPTGNLHLGNYLGAIVNFVKLQETHNCIYCVVDMHALTQPVEVWGGPTELARNTREVTAAFIASGIDPKKHIVFNQSQVSGHAELAWIFNCVARVGWLNRMTQFKDKAGKDREAASVGLYDYPVLMAADILVYRATHVPVGEDQKQHLELSRDIAQKFNNDFVDSIRGHGFNDGLFFPLPEPVITGPATRVMSLRDGTKKMSKSDASDNSRINLTDDADTIAQKIRKAKTDPEPLPSEEKGLEARPEAENLVGIYAALSGRSKQEVLTEFGGGQFSSFKNALVELCVAKLAPIASEMKRLVADPGHVDSILIDGADRARAIAAETMKAAKDIVGFIRPR

SEQ ID NO: 27 - protein, 164 aa, Unknown
  Bradyrhizobium species
MTTQRRSYETGHNPKCLVIVDDTAEWDRAVYYASRWAMRVGGGVVMLRIIELEDQNQQWLGVADIMRAEAEDAANEALDRAAGRANGIAAITPERVIREGDPTEQILDVIDKDVDISMLVLAANPGPEGPGPLISMMAQAAGSFPIPVTIVPGDLSDADIDGLS

SEQ ID NO: 28 - protein, 189 aa, Unknown
  Bradyrhizobium species
MFIQTEATPNPATLKFIPGRTVLDTGTMEFANREAAARSPLAERLFAVQGVTGVFYGADFVTVTKDDSEWQHLKPAILGAIMEHYMSGAPLLADGTAGNDEALDEEDEFFSEADAETVATIKDLIETRVRPAVANDGGDITFRGFKDGVVYLNMKGSCAGCPSSTATLQHGIQNLLRHFVPDVVEVRPM

SEQ ID NO: 29 - protein, 232 aa, Unknown
  Bradyrhizobium species
MLILAIDTALDACAAAVLDTDASKIIAQESQAMKRGHAEALMPLIARVMKASGIAFAALDRIAVTTGPGSFTGLRVGLSAARGIALAADKPVVGLTTLTAYAAPFVGEHSEHPILSAIDARHDHVYFQVVSGDGGSLIKPKVAPIEEALEASCFGVPHLVGNAAKILADRWPSDAPAPIRVDAQPAPDISWVAWVGAAVDPETAPARPYYLRAPDAKPPKDPLPNASQPSAS

SEQ ID NO: 30 - protein, 161 aa, Unknown
  Bradyrhizobium species
MMTWLSEWWGGGTAVVEAARPRDAARLAQLHGASFHRGWGEGEFETMLTERNTLVHRLRLGRKVIGFAVSRMAADEAEILSIAIDADQRGRGLSRNLLLTHLGHLAGRGVRSVFLEVEENNRPARRLYEWAGFGVVGRRERYYQQAGGEQLNALLMRRDLS

SEQ ID NO: 31 - protein, 163 aa, Unknown
  Bradyrhizobium species
MAKHPCFALQEPETMPVLKSPPAHKNTGIEARCAATGMRMTEQRRVIARVLAESVDHPDVEELYRRCVAVDDKISISTVYRTVKLFEDAGIIERHDFREGRARYEQMPDSHHDHLINLRDGKVIEFTSEEIEKLQAEIARKLGYKLVDHRLELYCVPLDDDKS

SEQ ID NO: 32 - protein, 451 aa, Unknown
  Bradyrhizobium species
MNVYDAQRMVDTLAPEGFVETDRAEDADLVILNTCHIREKASEKVYSELGRLRVAKDEAARQGREMRIAVAGCVAQAEGQEIIRRAPTVDVVVGPQSYHHLPQLLAHAKSYGRALETEFPVEDKFGFLAPPKPDAIRARGISSFVTVQEGCDKFCTFCVVPYTRGAEVSRPVTKIIDDVMRLADNGVREITLIGQNVNAYHGEGPDGATWPLGKLLHRLAKIPGIVRLRYSTSHPRDVDDSLIEAHRDLPELMPFVHLPVQSGSDRVLAAMNRKHTADDYRRVIDRFRTARQDIAFSSDFIVGFPGESEGEFAATLALVMQIGYAGAYSFKYSPRPGTPAADMRETVSAAAMDERLVRLQELIDSQQSAFNKAMIGNTVDVLFERAARNPGQIVGRTAYLQPAHVMASPDIIGKVLPVTIASLERYSLLGELARPAARPTPSTLSPMTTGA

SEQ ID NO: 33 - protein, 351 aa, Unknown
  Bradyrhizobium species
MPKSASDSPSLAPSRKFDRDMQIPPETQVVIDFDDNRAASALVGPYGQNLAQIERRLGVVVDSRGNHITIAGSRDGCDAARRVLETLYAQAVQGHDLAQGDVEGAIRAVIAQGSLFEFDPKAAKNAFETINLRKRPVRARTAAQDSYIRALKRHEMVFGIGPAGTGKTWLAVAHAAQLFERKEVDRIILSRPAVEAGERLGFLPGDLREKVDPYLRPIYDALYDLMDARVVERALQGGEIEIAPLAFMRGRTLTNAVIILDEAQNTTSMQMKMFLTRLGENSRMIITGDPSQVDLPHGQTSGLAEAVRLLDGVEGISQVKFTAEDVIRHELVARIVAAYEGLPQKPATGKS

SEQ ID NO: 34 - protein, 194 aa, Unknown
  Bradyrhizobium species
MARSSAAGLPIAGRRGNPIAGMQNTGTKLSSALPLTEVLVVADCWQAEPDAEAVIHRAVRAAAEIADSDVGEAELAVMLTDDSGIQTLNRNWRGIDKPTNVLSFPALQPTGSATSDDVPRMLGDIAIAYQTTRKEADDEQKPFDHHLSHLAVHGFLHLIGYDHEKDDDAETMESLEQEILAQLGIPDPYADRVD

SEQ ID NO: 35 - protein, 379 aa, Unknown
  Bradyrhizobium species
MRTGWTDMPDSDPTHDNPRNTRNLPAVVQDGEVLRPAADNWLTRAIRTLFGWKPGSVRDDLQVVLDASTPDEVGFSAIERTMLRNILGLHERRIADVMIHRADIVAVKRDIPLGELMSLFESAAHSRLVVYNETLDDPEGIVHIRDLLAFMTAKARVGETGKAKRKKPFPAGLDLRAVDLALPLSEANIIRKLLYVPPSMRAIDLLAQMQASRIHLALVVDEYGGTDGLVSIEDIVEQIVGEIDDEHDSDEPPAIVRQADNSFIADARASLDDARTVIGEDFVTGEAGEEVATLGGYLVSHVGRLPVRGEVISGPGNYEIEVLDADPRRVKRLRIAVRKERPAARAQRESRRREAAPDSNSPQANDNAPPSSSDGAGSQ

SEQ ID NO: 36 - protein, 536 aa, Unknown
  Bradyrhizobium species
MIPASKFRTAGLATILAWGWKRAALALVAGALSALAMAPINAWPVLFLTFPVAVWLIDGAGAGRWRGVPAAAMSGFWFGLGYFVAGLYWIGYAFLVDAPTFAWLMPFAVVGLPAYLALFTAAGFGLARLLWTRDASRLLALAISLTVTEWLRGHLLSGFPWNSFGYALSEPLALAQTASLVGLWGMTFLSVAIFASPAVLIDGSSRGRKPWLAPVAALVLLAAMAVFGAVRLSLQPTLLTKVKLRIMQPNLQQDVKFNYAAKAEVMQKYLTLSDRASGPQSTGVRDTQILIWPESAFPFFLTREADAMAQIAELLPKGTVLITGSVRAPDLPPGVRITRAYNSIYVIDHDGSVLQVYDKLHLVPFGEYLPFQDWMEKLGFVQLTRVQGGFIPGTRRRTMEIPDAPRALPLICYEAIFPGDIAARDDRPGWIINLTNDGWFGISTGPYQHLQQARLRAVEEGLPVVRAANTGISAVIDPSGRIVAQLGLGIEGVLDANLPQALPPTIYARIKDIPAAVIMAAALLLVIRRRFARRKA

SEQ ID NO: 37 - DNA, 259 bp, Unknown
  Pseudomonas specie
aatgggcttctgtgtgatgatggttgagatgtgtataagagacagatgtcgaggtgcgccacatcggcgaccaggtacgcaagatggtgcaggaagcgctcgacgccttcgcccgcttcgatgcggaccttgcgctgtcggtggcgcagtacgacaagaccgtcgaccgcgagtacaagaccgccctgcgcgagctggtcacctacatgatggaagacccgcgctcgatctcccgcgtgctcagcgtgatctgggcggt

SEQ ID NO: 38 - DNA, 6988 bp, Unknown
  Pseudomonas specie
gggtgcgctgggcggattgctggtgcgggtgcgcttcataccggttgaggactgatcgccttggcgtattcggtaacgcagtcgggcgccaacaccacctgattgcggccgccattcttggcttgatagagggccgaatccgccgctgccagcagctcggccggcgcgacgagcgcgggtgtcagcgtggcgacgccgaggctgatagtcagcgccaccggctggtcgccatgctgcagctgcaggttcgcgatcgtgcagcggatcgactccgccagggcgaaggcctgctcggcgtcaagccccggcagcaacacgacgaattcctccccgccataacgcgccgccacatcaccggcacgctggcaatgcgccgcgagcaccgccgccacctggcgcaggcactcgtcaccaaaaggatgcccatagctgtcgttgacccgcttgaagtgatcgatatcggcaagcagtactgacagcgggctgccgctgcgtctggcgcggcggatttcctcgtccagcaggcgatcgaaacgccggcggttggccagttgcgtcagcgcgtcggtggtgctcaggcgcatcagttcagcattggcggcctccagcccctgcctcacctcctccagcgctcgggtgcgctcaaagacccgagcctcgagcagctcgttggtgtgcagctggaagtccagcgccgcctgcttggcctgcagatgcagggcgcgctcttcggcaagcgactccgaggcctgcagggccagctgctgcgcggcgatacgtcgattgcgctcaacgttgatacgctcggccagcgccatggagagcaggacgaattcggtgaacatgccgatcagctggccattcagcgtccaggcattgagcggcagggtgccgttgagtgccagcaggttgatcaccgtgaacgccagcaaggtgctccaggcgatgctgaacaaccgcgccgacgggttgccgcgcgcccacagattgagcgtcaccgccatggtcagcacgcaatgcagcagcgcgacgtgttccatgaacaggtagcgcgccagcagcggcgccagcaggattgtcagggtgaccagcccccagtagcaggtcagcgtgatggacaccgtccagacccagccgccgtagcgtcgaatgccgagaaagcgcagggcgaagagcatcggcgtgaaaaagcacaacgccgcggaaagtccgtagaagcgcgccgacagctcgggtatttccggccacaggtagagctgaccgaagccggtgatggtcagcacgtagaacacggcgctcagcaggtagagcacgtagagaaaatagctgcgatcacgggtgaagatcagcaggctgcagttgtacagcacgatcaccaggatgccgccgaagaacaggctgaccagggtgacgtcgcgcaacttgccttcgatcagctgcttctcgtcgatcagctccagcggcagcgccaggatttctctggcctgcacatgcatgtagaccaccaccggctcacccggaggcagctcgagcggatagacgaagtgatgatcggccagcggacgcacgctcaatgccagcgcatcgccggcgagcatcgccgtaccccagcgctcgctctgcgggtaatgcagacgtaccgcgacgcgatcgagcactggccacttcagcagcaggtaccagcgctcggcgtcatcgccatggttgtgcagagtgaagcgcagccacgcaccctcgcgctgcttgccgaggttgggcttggtcagtatctgccactgccgatctgccgcggctatatcgacgaaatccgctgcgggctcgccgagcagatactccattgcaccgacctgcagcggcgcttcggtgcgcgtctgcagcaccggcgcctgcgcggccgccatcaccaaggcggtccacggcaacgtagtcaggcagactatcaacagcaagaagatgcggatcatgcgcgggcgtgcctcggtggcgctggaactgccggcaaactgccagcaaaccggcgccattatctaagcaccgacacgcgcatgatgccacctagccatcacgctgtcctgtccgggcgacagactgcttccttcagccccgtcgcctggcccagaactgctgcaccagctcgagcatgccatcggccagcagggcaacgccggttgcattgcgctggctgtgcagcccgctggcgacatggtcgtcggagctgccgcggatactgacaatgctctgactgatctcctcactggccgagctctgctggtcgacggccgcggcgatctggctgctcatcgcacagatctcgttgacccggctgttgatcccgctgagcgaatgctcggcctgatcagcatggctgacactgtgctctgcctgctcccggctgcgctgcatgacgtctacggcttcgcgggcgccagtctgcaacgagccgatcatccgctggatgtcggtagtcgccagggatgtgcgtgacgccagctggcgcacttcgtcggcgacaacggcaaagcctcggccctgttcgccggctcgtgccgcttcgatggcagcgttgagcgccagcagattggtctgctcggcgatgccgtggatcacctccagtacccggctgatttcctgactgtggttttccagctggtggatgacctcagcggcctgctgaatatcactggccaggcggatgatgctgctgccggtgcggccgaccacctcacgccccctggccgcttcgctgtcggctcgcgcagccacttcggcggtgcgctgcgcattcagcgcaacctcctgcacgctggcgctcatctgctggatggcggtggcgatcagatcagtctcctggtgctggcgtgcagcgctctgggtactgccatgcatggcgccaagcaggtcacgggcgtggtcgctgagctgccggctactgtcggcgatgcgtccgaccatcgcacccgcctcggtttccagcatcttcatggcgaattcgatctggccgatttcgtccttgcgtccggtatagatcagctggctgaccgggttgtcgccgacttgccgtgcctgccgcgccaggcgcgacagcggtaacagggtcatggctaccgccacggcgaccgtgccaccaagcgcaagccctgccagcgttgctgcgattggcgacgtagccagcacggtgcccacgatcaccccgccggcagcgccagcggcggcgagcagcgccacccgttcgcgcagatcaaccgtgccgcgtagtctcggcatgccgcggccgtcacgcagctgagcgtaaagcgcctccgccgcacgaatctgctcggactgcggacgggtgcgtaccgactgatattccttgaccttgccgtcacggctgaccggcgtggcaaaggcactcacccagtagtgatcgccgttcttgcagcggtttttcaccatccccatccacgagcggttcgcctgcagcgtttgccagagatcggcaaaggccgcttcgggcatgtccggatgacgcacgatgttgtgcggctggccaagcagttcatcttcgctgaagccggatatcgcgacgaaatcggggttggcgtaggtgattgtgcccttcaggtcagtggtggagaggatattcgcggtttcaccgacgacgacttcgcggccgctgaccggttggttgagacgcatagacagatcttccttggaaatgggtctgaatacgcaaggcaggactggcgccgcttctggtggcggcgctctcgaagaatgcgatcagcatcctgtaacagtggtgtgacgaatcggtgctcagtcctcctctttttcccccgcatcggtttccagttgttcaagcagatcgatcaggcgtcggcgcaactggccgagctcccgcgacttgaacgaacggcgggtacgctccagtatctggatggtttcttccagggcatcgcgcaaggtttgttcgctcagcattggttcttcttcgtgcactcatatgcaggttgggtgccgcttccgtcagtccgaaccggctgattttcaagccaaaaaaaaagacgcccggcgtggtacatccctggcggggtgtcacggcgggcgccttagctatcaggtccggttgtcctgagcggtaaagcaactttcgcaaaatgcgaaactggtggcattttgcctacgcctgatcgccggaacaagcctgtcgattcaggcatacaacggtagctctgccgatgcaccgttgcggcgcacggcagtacccatcaactgcgacggcgggtccagaactgttgcaccagcacctccatgctgttggccagttcggcgacaccggaggcactgtggcggctgcgcaggccgttggcgacgtgatgatcggaaccgcctcggatactcacgatgttctgattgatgttctcgctgaccgcgctctgctgctcgaccgccgcggcgatctgctggctcatttcactgatctcattgacgcggctattgatgccttgcagcgagcgcgccgcctgggccgcgtgctcgacactctgcatcgcctgctcgcggctgcgctgcatgacctccaccgcctgacgcgccccgccctgcagcgcgccgatcatgctgttgatttcctgggtcgattgctgggtgcgcgaagccaggctgcgcacctcatcggcaaccaccgcaaaacctcgcccctgctccccggcacgcgcagcttcgatggctgcgttgagcgccagtaggttggtctgctcggcaatgccacggatgacgtccagcaccttggtaatgtcgttgctgtgcgcttccagttcatgaatcacatcacctgcctgctggatatcctgggccagctggccgatggcggcccccgtgcgattgaccacctcggtgccggttgcggtttcgctatctgcacgactggcagcttcggcggtgcgctgggcgttgttcgccacttcctgcacgctggcggccatctggttgatcgccgtagccacctgatcggtttcgctctgctggcgggacgcgctttgcgtgctgctttccattgcaccgagcagttcatgagcatgctggctgagctggcgggaggcatcgccgatccggccgaccatggcgccggcctcggtttccagcattttcatggcgaaggcgatctggccgtactcgtcacgccggccggcgtagatcaactgacccaccgggttgtcgccaatctgacgtgcctgctcggcgagttgctgcaagggcgccagtgccaggtaggccaagccgccggcacccaaggcggctaccgctgcggtagccagggcgatggcaaagccagcgccgcccagcaggccgcccgcaacgacgcatggcgctgcgcccaacgcggcgaacaatgcgacccgactgcgggcactcagcggcttgcggcgcaatgcggccggtgcgcgcttgtcgcgcagctgcgcatacaactgctcggcggcggcgatctgggccggttgcggcttggtgcgtaccgactggtactccatgacccgcccattgcggctgatcggcgtgacgaacgcactgacccagtagtgatcaccgttcttgcagcggttcttcaccatgcccatccaggacttgccgccacgtacgctgccccacagatcggcgaaggcttcggtgggcatgtccggatgccggacgatgttgtggtgctggcctatcagctcgctttcttcgaagccgctgatcttgatgaaatccggattcacataggtgatgtcaccgttgagatccgtggtggagaggatattggcggtctcgctgaaggaaacgtcgcggccggtgaccgggagattcatgcgcatggcgggtctcgtgaacaaggcgaacggttgttctattccgccgaggacgtctcgatcgatgcaccacggtgcttcgtgcgttccttgcgttgctcgccttgcgacgattccctgctgtagacggccaagcgggtctgtgtatcgtttttgttcttatgcgacggggcgcaaaaaaagcgccagcaatgactcacctcctgcggtgatccctgttggcgcttcgttcgtcaggcccctggccctgtccgacagcacgccttcatgcctgaagactgatggcatgatgctatcgctcaggaaaccccacaatgcgactgaagtcctacgcctttggtcggtttcttcagccagccatgaacgggcacatcctatacaagaaagacaagatgtacaacttgtgaacaataaaatacggccagccgcacgcaaccactgccagccgatccggtcctagctgtgggcgcggaaactaacggatcggagagacgggtccaaagctccgggctgttcccgagccgggcgcctgctagactcaaattccgcgcagactcacaagggatgcccgggaatccgaaaacaaccaaaaaggagtgcatccatgaaaggcaaatccttggcatggatctttaccgctgctctggcgggaaccggtctgagcggagtggtacaggcgcaagagaagttcgtcaccatcggcactggcggccagaccggcgtttattacgttgccgggcaatcgatctgccgcttcgtcaaccgcaatgcggaaaacatcaagtgcaacgccccggccagcggcggcggcgtagccaacgtcaacggcctgcgcagtggcgaattcaacttcggcatcatgcaatccgaccaccagtacaaggcaatggaaggcgtcgcccccttcgagaaagaagggaaaatggacgacatccgtgcagtgttctccctgcagagcgaggttttcaccgtactcgcccgccgcagcgccaacatcaagggcctcgacgacctcaagggcaagtgggatccccgggtaccgagctcgaattcgccctatagtgagtcgtattacaattcactggccgtcgttttacaacgtcgtgactgggaaaaccctggcgttacccaacttaatcgccttgcagcacatcccc

SEQ ID NO: 39 - DNA, 3316 bp, Artificial Sequence
  biosensor construct
ctgtctcttatacacatctcaaccatcattcacacaggaaagtttctaatgaccatgattacggattcactggccgtcgttttacaacgtcgtgactgggaaaaccctggcgttacccaacttaatcgccttgcagcacatccccctttcgcaagctggcgtaatagcgaggaggcccgcaccgatcgcccttcccaacagttgcgcagcctgaatggcgaatggcgcgcgggcggcagcgaaggcggcggcagcgaacatcatcatcatcatcatggcagcgaacgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactaccctgacctatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaagtaagggcccaagttcacttaaaaaggagatcaacaatgaaagcaattttcgtactgaaacatcttaatcatgctaaggaggttttctaatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataataattcagggttgcaaccatcatcgatgaattcgagctcggtacccggggatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaagatgtgtataagagacag

SEQ ID NO: 40 - DNA, 8139 bp, Artificial Sequence
  Plasmid pCC1fos
gcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcccacgtgggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgat

SEQ ID NO: 41 - DNA, 191 bp, Unknown
  Pseudomonas specie
atcctatacaagaaagacaagatgtacaacttgtgaacaataaaatacggccagccgcacgcaaccactgccagccgatccggtcctagctgtgggcgcggaaactaacggatcggagagacgggtccaaagctccgggctgttcccgagccgggcgcctgctagactcaaattccgcgcagactcacaag

SEQ ID NO: 42 - DNA, 29 bp, Unknown
  Pseudomonas specie
tagactcaaattccgcgcagactcacaag

SEQ ID NO: 43 - DNA, 46 bp, Unknown
  Pseudomonas specie
atcctatacaagaaagacaagatgtacaacttgtgaacaataaaat

SEQ ID NO: 44 - DNA, 28 bp, Unknown
  Pseudomonas specie
atcctatacaagaaagacaagatgtaca

SEQ ID NO: 45 - DNA, 42 bp, Unknown
  Pseudomonas specie
tatacaagaaagacaagatgtacaacttgtgaacaataaaat

SEQ ID NO: 46 - DNA, 28 bp, Unknown
  Pseudomonas specie
tatacaagaaagacaagatgtacaactt

SEQ ID NO: 47 - DNA, 28 bp, Unknown
  Pseudomonas specie
aagacaagatgtacaacttgtgaacaat

SEQ ID NO: 48 - DNA, 28 bp, Unknown
  Pseudomonas specie
aagatgtacaacttgtgaacaataaaat

SEQ ID NO: 49 - DNA, 8 bp, Unknown
  Pseudomonas specie
ttatctaa

SEQ ID NO: 50 - DNA, 8 bp, Unknown
  Pseudomonas specie
cctatagt

SEQ ID NO: 51 - DNA, 283 bp, Unknown
  Pseudomonas specie
aacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaa

SEQ ID NO: 52 - DNA, 30 bp, Unknown
  Pseudomonas specie
aacatacgagccggaagcataaagtgtaaa

SEQ ID NO: 53 - DNA, 30 bp, Unknown
  Pseudomonas specie
tcgaatttctgccattcatccgcttattat

SEQ ID NO: 54 - DNA, 29 bp, Unknown
  Pseudomonas specie
tttaagggcaccaataactgccttaaaaa

SEQ ID NO: 55 - DNA, 28 bp, Unknown
  Pseudomonas specie
tttacggtctttaaaaaggccgtaatat

SEQ ID NO: 56 - DNA, 28 bp, Unknown
  Pseudomonas specie
ttgaatatgccttcaggaacaatagaaa

SEQ ID NO: 57 - DNA, 28 bp, Unknown
  Pseudomonas specie
ttgactcttttttatttagtgtgacaat

SEQ ID NO: 58 - DNA, 30 bp, Unknown
  Pseudomonas specie
attttgcttaccaaatacagcaatagtaat

SEQ ID NO: 59 - DNA, 30 bp, Unknown
  Pseudomonas specie
tttactcttgaagtgctccggggtgatagt

SEQ ID NO: 60 - DNA, 34 bp, Unknown
  Pseudomonas specie
agactacgattccatcaatgcctgtcaagggcaa

SEQ ID NO: 61 - DNA, 29867 bp, Unknown
  Bradyrhizobium specie
gtcagcgccgagatcaccgacttggtcgcagagaagccgaggatcggcgtatcgatgccgtagcccacagcgtagcgctcggcgacgacgtggccgtccttcacgatgacgacagccttggtcgcgcgtaacgggggttgttccggctcggcgaacgcacgatcgagcgccgcggccagttgcggattaacaggcgcgaccggcgcgggccccgcgatatcgggcaacagcgccggcaagggtttcgcatcgggtggcggcaccgagagatcggccacggcatcaccatggtcgagatagcagccaaaaccttcgcgatagaccgcgtggctgcgactgagaccaaggaatgtcaccgtggtatccttgcgcgcgcggtcgacatgatagtcgagcgcccaggtgatcaggccggcgccgggcatcgcggcggtggtctcggcaaagatccgatccggctgctggccggacacaaaagtttccgaacagaggatgttggcgacaaagccggtggcgaccttcggcaggtcgcgcgcccgcgcggctgacagagccagcgcactaagggcggtagtggtggcgaggatgagaagcaggtttcgtctgttcatgttggcctccggcatgacgcatcagcgcgtggcggccatgttgacgaagcgacccctctacgctcgccggatttcaaaatcgggctggccgaaaccacaccggcctcaccgatttcaaaattacggtgatattgcaatgatttgcgtctcaggcgacaggagcgttgagtcgccggtattcggtcggtgtcacgccggtggtcgccttgaatgcccggttgaacggcccgagtgactggaaaccggcgtccatcgcgatggtgatgacggggacttcggcctggctcggatcggatagcgcggccttggcttcctcgatccggtgattgttgaggaagacattgaaattgcggtagccgagccgctgattgatcagccgccgcaatcggtattcggggatcccgagcttggtcgcgagcgtaccgatcgtgatgttgtcgtgacggtagatgcgctcatcgcccatcagccgcatcagcgcatcgatcagcttctgatcgtttgcgtgctctgccgccgtcgattgagccgaaacgagcacgacgggtgcagcttcaggtgcagccggaaacaggtcggcgccgtcgacgcgcatcatcgcgtagcaaatcgccgccacgacaccggccagaacggcggaattcaccgtgttggcgacctcagtagtcgccccaccgggtaacagtatctgcaacacggcgttcatgccgccatacaatgcagaagcaccgacgatgaagatcctgacgcggcggcggcgctcgaccagatccgccgaccacgaggaaatggtctgggtcaccgccattgtgatgaagccgagcgccagcagattgatcgcgatgatggagatccgtgggttgcccgccggtgcaatccacatgcaattgacgaagctgaacgtggcaaccgccgcccagaccagcccatgccataagcgcaatttgaaggcatcgtcaaacagcgcgcgcgtgaacagccagaacaccaccacattgccggtcgacagcgcgatcaacggcgcgtgtccgatcgagaccggtgatgtcgcgccgatcgaatagctcacggcgtgcgccgccgatcccaaggcaaaggctgccgccagccgtccggccagcactgtgccaaagccgcgaaacagcgccgcggccagcaccagcagcagcgctacggtggcacctcgcaatgcgatgtcgagagtaggaagcagcatcaagtgagtccggtccgcgcgatgatgcgcgtcaggcgtatttaatccagatcggcgatcgattcaattttggaccgcccacgacgcgcgcgaggtcacccagatgtccgcggcatggccgaacagatcaacttctcgatccttccttgccccaagccgccgggcgacggcttgcgatggctcattcaccaaatcaatgcaatggatgatctcgtcggtctcaaaggtcgcaaatacccagtcgatcgaggcccgcgccgcttccaccgcgtagcctttgccgcggtactccctggcgatgccccagccaacctcgaagccgggccagcccggcggcgaccacggccccacccgcccgatatatttgccgctcgacttttcttcgaccgcgaacatgccgaagccatgcagcgcccaatgcccggacatcacggcggcgttgcgccagccagcgagttcactcgtgatcggcttgccgtcggccgcgatgaagcgcgcggtaccgggatcggacagcatcgccgtatttggggcaacatcatcgctgcgccattgtcgcaggatcaggcgcccggtttcgatcacggggccatcgacccgcatcagtctggcgcctggcttgagcgaagggatcatcggtttcctcatcctcaatacgtgtttggacgcgaccctgccattgttgctatgatcgctgcaaatccaaaaggagttttccatgagctggcaaccttccaatgatcccgtattgggcgatccgaaatcgtgcgacgcgctcgaactcatcatcgtgccgcgcacgcgcgatctcggcgacgggtttgaggtgcggcgcgcgctgccgcacggcaagcggcagatggtcggccccttcatcttcttcgaccatttcggaccggtgcagttcatggccggcaagggcatggacgtccgcccgcatccgcatatcgggcttgccaccgtcacctatctgttcgacggcagcatcatgcaccgcgatagcgagggtaacgtccaggagatccagcccggcgcgatgaacctgatgaccgccggccgcggcattgcccactccgagcgctcgcccgacgtgcagcgccgcgaaggccagaagatgctgggcctgcagagttggatcgcgttaccggcaggctcggaggagatcgcgccctcgttccagcactacgccgccgaaagcctgccgaccgtcaacgacggcggcttcaccgcgcgcatcatcgcaggcagtggctttggcgtgaaatcgccggtgacgatggtctcgccctggttctataccgaggtgacggcggttgccggcaccagcgtgccgctcgatgccgatcacgaggagcgcgcgatctatgtggtcgagggtgaagtcgagatcgcgggcgagcgatacgaaagcccgcggttgctgattttccggcccggcgaccgcatcacagtcaaggcggtcaagccgacgcggatgatgttcctcggcggtgacgcgctggaaggcccgcggcatatctggtggaatttcgtctcctccagcaaggagcgaatcgaacaggccaaacaggactggaaaaccggccgttttgcccatgttccccaggaacacgagttcattccgctgccggagtgagctaaccctcgcctatcgatacgccgccgcccgcgccagcccgcgggcgcgccttcctgaaagcaagacccaaatgaacgcgatgctcgccagcgacctgcctctgccacggatcgggcgcggcaaggtccgtgatatctatgccgtcggcgacgaccgcgtgctgctgctcaccaccgaccgcatcagcgccttcgacgtcgtgatggccgaaaccattccgatgaagggcgcggtgctgacccagatcagcgcctggtggttcaggcagctcaaaggcgtggtgccgcaccacatgatcagcgccgatgccgacgagatcatcaagctggtgcccgcactgaaggaccatcgcgcagacattgtcgggcgcgcgatgctgtgccggcgcaccaccgtcttcccggtcgaatgcgtgatccgcggctacatctcgggttcggcctggaaggaatatgccgcatcgggcacgctcgccggcgaaaagctcgcggctggcctggtcgaaagcgaaaaactcgagcccgcgattttcagcccggcgacaaaggccgagaccggccacgacgagaacatcacggtagcaggcgtgcgcgaaatcctgggggccgaggtcgcgcaaacgctcgagcggatggcgcgcacggtctatgatttcggcgagaagatcagccgccgacagggcatcatcatcgccgataccaagttcgaattcggccgcgacggcgatggccgcgtcatcctgatcgacgaggtgatgacccccgacagttcgcggttctgggcggtcgacgtctacaagcccggccagccgcagccgagcttcgacaagcagccgctgcgcgactacctcgacaccgagcgccgcgccggccgctggaacggcgacgcgccacccccgccgctgccggccagcgtcgtcgacgccaccagcaagcgctatctggaggcctatcggcgagtgacggggaatgcgcttgcgatctgactccgaagcagccgtcattgcgagcgaagcgaagcaatccattgcgcaacagaaagtatggattgcttcgtcgctccgctcctcgcaatgacgagtaatatgcgatcgcattacccatttcgctcgaaaacgctgtaggctcgccccatcaaagcgaaatggaggacgcctgacatgaccgagaccgacaatttgctggtcgaacgcgatggcccggtgaccatcgtttccatcaaccgtccgcattgccgcaacgccgtcgacggcgccaccgcgcgcaaactctacgatgcgtttctggcgttcgacgccgacgaggcggcgtcggtcgcggtgttcaccggcaccggcggctatttctgtgccggcgccgacctcaaggccgtggcatcgggcgatccgaacaaaaagcgcgagatcggcggccacgacagtatcgcgccaatgggcccgagccggctgcggctgtcaaaacccgtgatcgccgcggtcgaaggctttgcggtcgccggcggcatggagctcgcattatgggccgacatgcgcgtcgtcgccgaggatgccaccttcggcatcttctgccgccgcttcggcgtgccgctgatcgacctcggcaccatccgtctgccgcggctgatcggccattcccaggccatcgacctgatcctgaccggacgcccggtcggcgggccagaggcgttgcgcatgggccttgccaaccggctggtgcccaagggagaggcccgcgcgcatgccattgcgctggcgaaagacatcgcccgcttcccgcagacctgcctgcgcgccgataggctgtcgacgctgcggcaatgggacctcgaggaggaagaggccatcgccaacgagatgcgcggcggcctcaaggtgattgcgtcaggcgaaaccttgtcgggtgccacgcgatttgcctcgggtatcggccgccacggcgcgttcggcggtgatgtggtggggaacggctgaccatcgtgtccggcggttggcggagtgccgattccgccggccgacccaccatgaattctgttgtttccgaatgccacagtcgtctaccactgggatcggcggtgggggtaagcgcaggtggccagccaactattccatttgaaaccgggtatacgcccggcgcttagcgacatcatcggcggcagcgccgccagcgtcctcacggtgacattcggcctgtcatacgcgctgctgattttcacgggccccctgacgccgtacctgtcctacggggttgccgcgacctttatcgcctcggccgtcctcggcgcggttgtcgcgctcggaagttcatttccattcgcgatcgcagcccccgagagttcgacggcagcgatgaccggaatattggcatcgtcgctggtcgagcacatgacgacgcgcgatccatcggcatcgctgctggccccggttctcataacgctcggtctttcagccattatcacgggcatcgtgctgtgcggcttcggggtaacgcggatgggccgtgcgatccgctatgtgccctatcccgtcgtgggtgggttcctcggtgctaccggctgtctgatcgttttgggagcgatccgcgttatcaccggccaacgccttcaattggcgacgctcgatcggttcgccaatatcctgacattgtccgaactggcggcagcctgcgccatggcgctggtcctgtatttgacgtggcatcgctcccgcaccgcgttcggcctgccggcgattctggtcggcggcatcatcgttgcccacatcgcgttccggctggcgggcatcacctcgaccgaggcgcaggcgtccggatggacatttcagccaccgccggccgtcaacatcatgttgccgtggagcgcagctgaaataagccactatccctggtacgcgctgccggacctgatcggcaatttgatcgccgtgatctttgtcaccgcgtcgagcacgctgttcaacaccacgggcgtcgaagtggcttcgcaccgggaagccaatctcgaacgggaattgaacgttaccggcctcgccaacatcctgtcgggcacattcggaggcttcaccggctgcatttcggtcagccgctccatgctcaacctgaacagcggcgggaccagccgactctccggtctgaccggcgccgcgatctcgacgatcatgctggctgccgcgcccgcgcttctcggttacatgccgaaattcgttctcggcggcttgctgctctatctcggcgccgaccaacttcacaaatggatcgtccagtcacggcgccgcttgtccttcatcgaatatctgtcgctgctcgcgattataatcatcatcgtgcaatggggcttcatcgccggcaccctgatcggcgtcgtgatcggctgcacgacctttgcgctgagcgcctcgcgcatcgattccatcaaattcggtttcgacggatccgagtatcgcagttcgctcgaccgggcacgcgacgatcaggcggtgctctccgcgcacgggggaaagattcagggcctgaacctgcagagctatctgttctttggctctgccaaccggctttacgagcgtgtcaaggcgctgctcgcacggcaccccgaatgccgttttctgatcttcgacttcaagctcgtcaccggcatcgattcgtcggccgcctacagtttcgctcagatcaagcggacagcgcatgatgtgggcgtcaagctggtactggtgcatctgcctgccgcggccgagaaggcgttgcgatcgagcgagttcctttcgagcgaggtgaacatcgtgcccgaactggaccacgcgctcgagtggtgcgagaacgagatcatcctgcagcacaagggcctggagcgtgaggaagccagcctgcgcgactggttcacggagatactgggcagtgaacgcgacgccatggaactgatccaccgctgccagcgcctcgaggtcgaggccggcgaaattatcgtcagcgccggcgatgccgccgattcgatgcatttcattctggacgggcgcgtcgggatcatgatttccaacggtgagggtggcagcacgcgggtgcgaagtcttggccgctacacgacgatcggcgagatgggcctggtttcgcgttcgccgcgcagcgcgacgatccaggccgaggtcgccagcacgctttatgtgctgaccgtcgagcaattcgaggcgatcaagcaagacgaccctgcactcggtcagaagcttctgacctatttcgtctccgtcatggccgagcgcctgaccttcgccaaccgcatgatcgcggtgctgcggcgatagacgctcaggcctctgccacgcccgcgtcgcgcgagttacacccccgccatcatcacgtatttgatctcgacatattcttccatgccgtgatgcgagccttcgcgacccaggccactctccttgacgccgccgaagggcgcgacttcggtggtgatgagcccggtgttgacgccgaccatgccggactccagcgcttcggcgacccgccagacccggccgaggtcgcgcgaatagaaatacgacgccagcccaaacggcgaattgttgcacatcgcgatcacttcggcttcatccttgaagcggaacaccggcgccagcggaccaaaggtctcctcgtgcgcgaccagcgcgtcgggtttgacgttcgcaagcaccgtcggctcgaagaagctgccgccgagcgcgtggcgcttgccgccggtaatgatcttggcgccgcccttgaccgcatccgcgatgtgcttctcgaccttgtcgatggcatccatgttgatcagcgggccttgcgtcacgccggcttcggtgccgtcgccgaccttcatcgccgcgaccttgttcgagagcttttcgacgaactggtcgtagatcttgtcctgcgcgtagatgcggttggcgcagacgcaggtctggcccatgttgcggtatttcgagacgatcgcgccttcaaccgcggcatcgatatcggcatcgtcgaacaccacgaacggggcgttgccgccgagctcgagcccgagcttcttcacaccgacggccgcctgctgatacaggatcttgccgacttcggtcgatccggtgaagccgacgaagcgcaccgcgggatgctcgcacagcaccttgccgatcgccgaggaattgccggtgaggatgttgaacacgcccttcggcacgccggccttttccgccagcgccaccagcgccagcgccgtcaatggggtttcattggcgggcttgagcaccaccgtgcagcccgcagccagcgccggcgacactttgcgggtgatcatggagcaggggaaattccatggcgtgatcgcgccgcagacgccgatcggctgcttgatcgcgagcaaccgcgcatccgcacgctgggtcggaatggtttcgccatagacgcgccgcgcctcttcggcaaaaaattcgacataggcgccgccgatgtcgacttcaccgagtgcttccgccagcggtttgccctgctccgaggtcaggatcaaggcgaggtcttcgcggttagcgatgatcaggtcgaaccatttgcgcaaaatgttggagcgcgctttggcggtgagcttggcccaggccggaaacgcgcgcccggcggcttccacggcttggatggtttccgccgtgctcatgaccggaattttcgcaagctcgatgccattgaccggattggtcacgggcctcgatgcctttccagtccaggcgccgtcgatgtagcactgctcgcgcagcagcgacgaatccttcaggcggtcgcgcagcgagggggtggaagattgcgaagcacgggcggcaacgggcgggttcatggcgttactcctcggattctgggtattgcctccgaatataggccgaagcccctcacaatgcatcggccgcaaatgcgcggctgattggacctattttgagggtttgtcgcggtgtgttcaggccgcgccgttcatgtaggtttcgcggcggccgatcatgcgttcggcggccgccttggcgtcggcgcgggaggaggtcgcgcaggtccggtattcgagatcgggcagttttgccgggtcgcggcgcaagaaccaggatttggaaccgaccggcagctgcgccagcgcctgggcgacattgtcgaccgccgcgaaggaatgcagcgcaccggtgccggcatagcggacttcatagaggccgggcgagaccggcgcctcgatgttttcaccgcgtccggcgcggggatatcgcttccattcactccaggtcgaaatcatcgcgttaccctcgcaatatcgaaagctacaaccattttcaccaattcgtgcagccggggaggcggcatttccgttgtaatctgaatgtcttactgtaaaaaatcggcacaggaaatccgtgtgaaatcacgttatcgtggattgccccacgcggttaccggtattgcgatcaatcgaccgtggactgtcgcgacgtgttgcggtttgtgtcgcccgcgtcccaagccctctcccgcaccatcggcccatcacgcgatcgcgacatcggcgggccgccgcccgcgcttgcgggatcgcgctgccgggaggaagatcggtgcactccataaaaaataaaccggaggcgaccccatgcaaagcaaagctcagatcgatcagattctgcgtcagaagtgcgaggccaaggagattcccggcgtagtcgcgatggccgcgaccggcaatgaggtgatctatcagggcgcgttcggcaaacgcgacctgtccaaggacgatccgatgaccaccgacagcgtgttctggatcgcctcgatgaccaaggcgatcacgaccgcggcgggcatgcagctcgtcgagcagggcaagctgtcgctcgatgaaccgatcggcaaggtgctgcccgatcttgcttccccgcaggtgctgaaaggttttgacgccaagggcgaaccgaaactgcgcccggcgaaaaagccgatcacgctgcgccatctcatgacccataccgccggcttctgctaccatttgtggaacggcgacatggcgaactatctggagaagaccggcacgcccgacattaccagttgcaagaacgccgcgttgaaaaccccgatcatgtccgatcccggcacgcgctgggaatacggcaccaacatcgatttcgtcggcaaggcgatcgaggccgtgagcggcaagcggctcgacgcctatttgcgcgatcacatgctcacgccgctcggcatgaccgataccagcttcaagctcggcgatgcgcaacgcaagcgcctggtcggcatgcatgcgcgcggggcggacggcacgctggcatcgatcccgttcgagctcgagcaggatccggaattccacatgggtggcggcggtctctatggcaccgcgggtgattacatcaaattcacccagatgatcctcaacaagggaaaaggcaacggcaaccaggtgctgaagcccgagacggtcgcgatgatgggccagaaccacgtcggcgaactcagcatgaccaaattgacctcggcggtggcgtgggccaccaacgatgtcgatctctatcccgggatcgagaaaaagtgggggctgagcttcatgatcaataccgccaagacgccggagggccgcagtcccggcagcctcgcctgggcgggcctcgccaacacctatttctggatcgacccggcgcgcaacgtcagtggcgtgatcctgatgcagctgttgccattcgtggatcagaagtgcctggaagcctacgcaggcttcgagcgcggcatctatgccgggctcgatgccggcagcgggcagaaggcggcgtaaggcaagttcgttagagatcgtcattgcgagcgaagcgaagcaatccatcccacggcataacggataggtggattgcttcgtcgctgcgctcctcgcaatgacggagcgcgctgggagattgctcccatgcatgtgatacgcgtaatgtccaattcgcgcgccccttcgcatggagaagcagatttgacggacattgcaggcagttacgtttgcggcacggccgatgcgccgctcctgggcgagaccatcggcgcgagcctcgatctcgcggtccagcgctggggcaaccgcgaggcgctggtctcgccaagccatgatgtcaggtggatatggcgcgaattcgctgaacgtgtcgatacgctggcggccggctttcttgcgctcgggctggaacgcggtgcgcggatcggcgtctggtcgctcaatcgccccgagtggacgctgacgcagtttgccgcggcgaaagccggcatcattctcgtcaccatcaaccccgcctatcgcctcagcgaactcgaattcgcattggcaaaagtcggatgcgccgcgatcgtcacggcgaccgccttcaagacctcgaactacatggagatgctgaacacgctgctgcccgagcttgccaaatccgtgcccggcgaattgcaggcggcgcggctgccgcagctgcgcgcggtgatccagatcggcgggccggcatcgcccggcaccatcgcattcgaggacgtgacgcgaatgggcggcgcgcgccaccgggacacgctggcggagctcgccaaaaccttgcagttcgacgatgcggtcaatatccagttcaccagcggcacgaccggatcgccgaagggcgtgacgctgacccaccacaatatcctcaacaacggctattttgtcggccgcgccatgcgcctgaccgaacaggaccgcatctgcattccggtgccgctgtatcactgcttcggcatggtgatgggtaacctcgccgccgtgaccctcggcgccaccatggtctatcccggcgagggctttgatccgctggcgacattgcagaccatcgaaaaggagaaatgcacgacgctctatggcgttccgaccatgttcatcgcccagctcgatcacccggaattctcgcgcttcgacctgaaatcgctgcgcaccggcatcatggccggcgcgccctgcccggtcgaggtgatgaagcgcgtcaacaacgagatgaacatgagcgaggtgacgatcgcctacggcatgaccgaaaccagcccggtcagcttccagagctcgaccgatgatccgctggagcgccgcgtctccaccgtcgggcgcattcatccgcatgtcgaggtcaaggtggtcgatctcgagggccgcgtcgtgccgcgcggcgaacgcggcgaactctgcacccgcggctacagcgtcatgctgggctattgggacgaggcggaaaaaacccgggacgtgctcgacgccaatggctggatgcacaccggcgacatcgccatcatcgacgccgcgggctattgcaacatcgtcggccgcatcaaggacatggtgatccgcggcggcgagaacctctatccgcgcgagatcgaggaattcctctatcgccatcccaaaatccaggacgtgcagattttcggcgtcgccgatgatcgctacggcgaggagctctgcgcctggatccggacgcggccgggcgagacgctgaccgcggaagaggtccgcggtttctgccaggggcagatcgcccacaacaagattccgcgctatgtcgagttcgtcgacgaatttccgatgacggtgacgggcaagatccagaaattcctgatgcgcgatgcggtcgaggcgaagttgggattgaaggcggcgaagacagcgtgagctagagatcgtcattgcgagcgaagcgaagcaatccattctctctttccgcggcgccatggattgcttcgctacgctcgcaatgacgaacaaacacaccttcgcgatcccgcggcacgctgtgcccgaggtttgcaagtaatcttcgccctcaccaaaatcagagggcgcagggaatgccggatgcgcgctgcacccgcggtctcatgcgcaatgtgcacagaaagtgcgcgcatgagcatacagggcagcggaggcaatccgacattccctgcgcaatggctttacggcttataacgagctctccctggtgatcggctttcttgccaccatcgccggcggaaatttctttccgccgacttgacgccagcaccggggcgtcaggaccacacgtcttcgccgtacgcaatccgcccgttcgtctcacgcgcgcatcgcgtccaccgcaacccgtccctcgttggtaacgacggccgacgcccctctgagcgggacgggatggcaacagatatgccctgtttcgcgcttcgggaaaagcgaaatattttttattttagggcttgacacgatttccgataaccggaagtaatttgcccgtcgggtcaatttgtcgcgccttctctctccatattgtcgtcgcgagtgaaacgacgcagcaggtccagcgttcattatcatttgtcggagctgtcctcgccgtcttcgttcgcaggcttgagtcgaacgccgcggcgcgccagtgcctcgcgaagcagaatctcgatttcggcgttgacgctgcgcaagtccgccgcggcgcaacgcttgactgcctcgagaatcgctttctcggcgcggagcggaaaggggacgcgttcgggtccgggcggccggcctcgttgagccatggccggtcacgagtacagagtgccggtattgatcacgggcgagacctctctgtcgccaaccaggaccacgagcaggttggagatcattgcagccttgcgttccggatcgagcgcgtccgcgagtttcttccggtcgagttcgttgagggcatcttccacgatactcacggcgccccgcacgatcatgcgccgggcggcaaccacggcgctggccgcctgcttccggagcatggcgctcgcgatctcggacgagtaggcgaggtgcgagattcgcgcttcaagcaccgcgacgccgatcggctccatgcgtgcccggagctctcccagcaaggcctctatgacagcgtcgccaccgtcgcgcaaactgaccgccgatatcgccgcacttgccggccgatcgccctgccgctccgcttcctcggtctcgacctgatcataggggtgcacgctcaccatccgccgcagcgcggtttcgctctgcgactttacgtaactggcgtaggaggcgacctccagcgacgcctttgcggcgtcctccacgcgccagacgatgaccgcaccgatatcgatcggattgccgacggcatcattgaccttgagtgggccgctctccagcgtctccagacggcgcgagactttatgcttggaattgaaaggattgacccaccaaaaaccaggccggtgctcggtgccgacgtagcttccgaacaacaggcagacaaggctttcgttaggctgaagcacaacgaagcccttgagaacgccgatggcgaggagagcggcaatgatgaccggtgaaagagcaatccaggcgcgcaccagcgaggagttgttggggtcgaatgcgatccaggaaagacagcaaagaaacacgatcccggccaagccgagcgcaagcatcacatagccgttgacggtatggaactgcttgtcggcaaacgtgctgcgaactggtggggacgaaatggtcgtggctgacataatgggcctccgttcacaattcgtgatatatttatgatatcaaataatcgacgcgagtcaagcgcgttcgcttgcctaacaaatttgaaaatgagtccgttatggccgggcttgacccggccatccacggctttcttgcgtgatgctgccaagaacgtggatgcccgggcccaagcccgggcatggcgagtgaagagattgcgccgtatcgcgccagccctaaaccggcagcccgttctgctgcgccaattccttcagcgacacctgcggccgggcgccgatgtgctggatcacttcagccgcggcgagcccgccgagtcggccggcggcctcatggccggcgttgcgcaccaggccgaacaggaatccggcggcaaacagatcgccggcgccggtggtgtccacgagtttttgaatcggaaacgccggcaccgcgacgacgccgtccgtggacgccaccacgcagcccttctcgctgcgggtgacgacgccgagtttggtgtcggcgcgcagctgcttcagcgcgatgtcgaaatccgaggtctggtacagcgagtgcagttcggcctcgttggcgaacaccaggtcgaccgtgcccttgcgcatcaaatcgagaaattcgtcgcggtagcgatcgacgcaaaatgagtccgacaacgtcagcgcgacctggcggcccgcgccatgcgcaatggtcgccgccttgacgaaggcctgcttcgcgcttttcggatcccagagataaccttcgagatagatgatgctggatgccgcgatctgcgcctcgtcgatatcggacggcatcagctcctgcgccgcgccaagataggtgttcatggtgcgctcgccgtccggcgttaccagaatgtaggagcagccggtggccggaccgcccgatgccgctttggtttcgaaagctacgcccgcggcgcggatgtcatgggtatagaggcggccgatctgatcgtcccggaccttgccgacataggccgcgcgcgcgccgagattggcgatgccgacaatcgtgttggcggccgagcctccggacatttcggtcgcaggccccatgtctgcgtagatcgcggtggcgcgcgcctcgtcgatcagcgccatgccgcccttggtcatgccgtggcgggcaagaaagccctcatcggtctgcaccagcacgtcgaaaatcgcgttgccgattccgagaacgtcgtattttgcgtcggccatctcaccgtcctgtttaccggcagccacgaaaggctgcggcctatcacgaccagtgcaaggcgtgaacccgtaaattcagattggtcggcaccgtctgccgacaattgaaaagtgcctcgataccaaaaccgccggccccgcgcggcgtccgagatttttgtcgtgctttcacactattcgagggaagttgccagattttccagcggagccgtcaggcggcaaatgagtccggttgggccatacgacacttcaaccgagcctccgaaatcgtccttgagcacccgtgaaatgaggcgcgaaccaaagcctgcaccagcgggttccgacgccggcgggccaccaacctcccgccactcgaaacagaattttggctgaccaacctcatcttcggatgtccacgtaatcgacacatgaccgctcggcaccgtcaaggcgccatacttcaacgcgttggtcgcgagttcgtggattgccagcgacagagcgagaccctgtcgcgatttgacgacaagtgggggacctgaggtcacaaaccgaccttcgccggtccgataagggatcagggcggtgctgatcacatcctgcagcggcgctgcggcccaatttgccgcggtgagcaagtcgtgcgcccgcccgaacgcagcaagccgtccatgatatttttcgagatcggccttgctcgatacgtcgcgaaatgtctgggttgccacggcaccaagcacggtgagcgtgttcttgacgcggtgcttcagctcctcattgatgagcctcagcgcttcctttgacatgtgctcggacgtgacatccttgcagacgacgagcacgccgccgaccttgccttcgagatcgatcgggccaaagctgtacgtccaccaaacatcctcgcgacgaccgtgacgcgtgaccggcaccagttggtcctcatgccaggtcgctcctttacccgccatgacgtattcgatctggggaccaatgatgtcccatatctcatcccagcactcgcggccccgcgctcccaaagcgctcggatgcctttcgggccccatcgtctcgcggtaggcatcgttatagaactggatgagttccgggccccaccagatgaacatcggatggcgcgaagtgagcactaaacgcaccgtaacccggagactttgcggccatgtttcgggcgggccgagagacgtttttgaccagtcgaatcctcgcgtcagagcgcccatctcgccgccccccgccaagaagtcgctgactacctccccgcccatccagaaaacctcactcagacgcgcctggccaagcctatagccggagttcggtgatgtgcaatgttcagcttggtcagacttggacacctgactgagagacgtccgcttacccccgatggccgtcgtcgatcgcgattgatcccgggcgccgccgaaggcgaggctcgccgtggttgctgcgtttcggtatggttggaaaatggggcgaatgtggctgaacatatcgggcttgattatctgcctcgtcggtttcgatgattgccgtcgatcgtcccgatgatccgctcgttgctcacggtctcgacgggaacgctggcctcgcgcctgctcggattcgtgcgcgattcgatgatcgcagcattgctcggcgccggtccggtcgcggatgccttcctcgcggcctttcaaatggtcaacgtcgtgcggcggctactgaccgaaggtgggctgaatgcgtcgctggtgccggcgtggctgcgcgtgcgcgacaccgacggggccgcggcggccgcggccttcgccggccgcgtgttcggcaccatcacggccgcgctgttcgcgggcacgttgctgctcgcgttgctgatgccgctcgtgctgatggtgttggcgccggggttctccggaggggaaacgctgcagcttgccgtcgacgacgcgcgtctgatgctgccgtatctcgcctttgcgggtcccgtcaccgtcatgatggcgctgttgaacgcacaaggccgttttgcgctgacggcgttctcgccgctgttgttcaatgtcgcgctgatcgcagtgttgttcgtgctgctggcatcgaacgcgaatgccgtgcgcgccgcgcagatcctcgccgccaccgtcggcgtcgccggcctgctgcagctcgccatcctggtgctgcgccgcggcagtaacatcgcaacgccgcttcgggtttcattcgacaaggagatacgcggctttctggccaaggccgtccccggcatgatcgcgagcagcacgcctcaattgctgatggtcgctggcgcgattgtcgcgtcgtcatcgccatcctcggtgtcgtggctctatttcgccaaccggcttgtcgagttgccgcttggcatcgtcggcatcgccatgggcacggttttgattccggaaatgacgcgcgccctgcgcggcgaggatcgaaccgccgtcgcgcatgcggaatcgcgcggcctcgaactcgcggttggactggctctgccggcgacgctcgggctgatcctgctcagcgaacccgtggtgcggtttttgttcgagcacggcgccttcaccgctactgataccgccgcaaccgcgcacgcgttgatgtggctggtgctcgggctgccggccaatgtgctggtgaaagcattgtcgccggcgttcttcgcgcgcgaggacacgatgacgccgctgctcgcgatgctcaagggcacagcgttcgcacttgtgcttgccgtcctgctcggtcactggtttggcgccagcggcgttgccgccgccatcgcgctcggggcatggagcacggccttcacgttgatccgccggggcgccgagaccttcgggttttcgatcgatgcagccgcgcgaaggcggctgcccggcatcctcgcggccgcactcgccatgggcgcggcgctgtggctcgcggtgcgctggctgacgcccgtgctcgccacgagctcgcacggtctcgtcatcgtgctgccgatcctgattatcgccgggattgcgatttatgggctgtttctgcggctttttggcatcaccggctggcgcgaagcgatcaaggcgatcaggcaaaacagggccgccgacttgcgcgactagcacttaaatggcaaacgacggcgccaaagcaggaaattcgtgaggcaagcataccatggccatggttgaacgggttttttcaggcgtccagccgaccggcaatctgcatctcggcaattacctcggtgcgatcgtcaacttcgtgaagctgcaggagacgcacaactgcatctattgcgtggtcgacatgcacgcgctgacgcagccggtggaggtgtggggcggcccgacagaactcgcgcgcaatacccgcgaagtgaccgcggccttcatcgccagcggcatcgatccaaagaagcatatcgtgttcaaccagagccaggtttccggccacgccgagctcgcctggatcttcaattgcgtggcgcgggtcggctggctcaaccgcatgacgcagttcaaggacaaggccggcaaggatcgcgaggcggcttccgtcggactctacgattatccggtgctgatggcggccgacattctggtctatcgcgccacccacgtgccggtcggcgaggaccagaagcagcatctcgaattgtcgcgcgatatcgcgcagaagttcaacaatgacttcgtcgattcgatccgcggccacggttttaacgacggcctgttttttccgctgccggagcccgtgatcaccggacctgcgacgcgggtgatgagcctgcgcgacggcaccaagaaaatgtcgaagtcggatgcgtcggacaattcgcggatcaacctgaccgacgatgccgacaccatcgcgcagaagatccgcaaggcgaagaccgatccggagccgttgccgtcggaggaaaaagggctcgaggcgcggcccgaggccgaaaatctcgtcggcatctacgcggcattgtcgggccgcagcaagcaagaggtgctgaccgagttcggcggcgggcagttctccagcttcaagaatgcgctggtggaactttgcgtcgcaaaactcgcgccgatcgcctcggagatgaagcggctggtcgccgatcccggccatgtcgattccatcctgatcgacggcgccgaccgcgcgcgggcgatcgccgccgaaaccatgaaggcggccaaggacatcgtcggctttatccgcccgcggtagtacaaacttacccgcgggcacgatctcacctctcccatcgggagaggtcggcgcgcagcgccgggtgaggggttaggaaccatcgatagaccttatcccctcacccgctcgcaagatgctcgcgacctctcccgatgggagaggtaagaacactgcagttgcggcaagactcaagcaaacacaatagcgacgccgcttgtcgaaggcggctgcgccgtggcagcatgcgccggtttggcaagcaccgggacatgcatgaccacccagcgacgaagctacgagacgggtcacaatccaaaatgcctggtcatcgtcgacgacaccgcggagtgggaccgcgcggtctactatgccagccgctgggcgatgcgggtcggcggcggcgtcgtgatgctgcgcatcatcgaactcgaggaccagaaccagcaatggctgggggtcgccgacatcatgcgcgccgaggccgaggacgccgccaatgaggcgctcgatcgcgccgccggccgcgccaacggcattgccgcgatcacgccggagcgggtgatacgcgagggcgaccccaccgagcagatcctcgacgtcatcgacaaggacgtcgatatttcgatgctggtgctggcggccaatcccggccccgaaggccccggcccgctgatctccatgatggcccaggccgccggttccttcccgatccccgtcaccatcgttcccggcgatctcagcgacgccgatatcgacgggctttcctagtatttccatacagatacaggcctggaacggccgatccgccccttgatcgtgcgttggccgtcgccatctgctaagggaaccccacgcgccggccttgaaccggcgacgccggagaaaagcagatgttcatccagaccgaagcaacccccaatcccgccaccctgaagttcattccgggccggaccgtgctcgacaccggcacgatggaattcgccaaccgcgaggccgccgcgcgttcgccgctcgccgaacggctgttcgccgtgcaaggcgtcacgggcgtgttttacggcgccgatttcgtcaccgtgaccaaggacgacagcgagtggcagcatctgaagccggcgatcctcggcgccatcatggaacactatatgtccggcgcgccgctattggccgacggcaccgctggcaatgacgaggcgctcgacgaagaagacgagttcttcagcgaggccgacgccgagacggttgcgaccatcaaggacctgatcgagacgcgggtgcggcctgcggtcgccaatgatggcggcgacatcaccttccgcggcttcaaggacggcgtggtctatctcaacatgaagggctcctgcgccggctgcccgtcatcgacggcaaccctgcagcacggcatccagaacctgctccggcatttcgtgcccgacgtggtcgaagtccggccgatgtaggggggaaacgcgaatggcgagtagcgaatggcgaatagggattggttctgacactccattcgctattcgccactcgctattcgcccgcaccccatgctgatcctcgctatcgataccgcgcttgatgcctgcgccgcggccgtgctcgacacggacgccagcaaaatcatcgcgcaggaatcgcaggccatgaagcgcggccatgccgaagccctgatgccgctgatcgcgcgcgtgatgaaagcatccggcatcgcgttcgctgctctggatcgcatcgcggtgaccacgggacccggcagctttacaggacttcgtgtcggcctttccgccgcccgcggcattgcgctcgcggccgacaagccggtggtgggactgacgacgctgaccgcatacgcggcgccgtttgtcggcgagcacagcgagcatccgattctatccgccatcgatgcgcggcacgatcacgtctatttccaggtggtgagcggcgacggcggttcgctgatcaagccgaaagtggcgccgatcgaagaagccctcgaggcatcgtgcttcggcgtgccgcatctggtcggcaatgctgcgaaaattctcgccgaccgctggccttccgatgcaccggcgccgataagggtcgatgcgcagccggcccccgacatcagctgggtggcgtgggtgggcgccgcggtcgatccggagacggcgccggcgcgaccgtattatttgcgcgcgcccgacgccaagccgccaaaggatccgctgccgaacgcgtcgcagccatcggcatcatgatgacgtggttgtcggaatggtggggcggcgggaccgccgtggtcgaggcggcgcgcccgcgcgatgcggcgcggctggcgcagctgcatggcgcatcgtttcaccgcggctggggcgaaggcgaattcgaaaccatgctgaccgagcgtaacacgctggtgcatcgcttgagactgggccgcaaggtgatcggttttgcagtctcgcgcatggccgcggacgaggctgaaatattgtcgatcgcgatcgacgccgaccagcgcggccgcggcctgtcgcgcaatttgttgctgacgcatttaggccatcttgccgggcgcggcgtgcgctcggttttcctggaagtcgaggaaaacaaccggccggcgcggcgattgtatgaatgggccggattcggcgtcgttggccgccgcgagcgctattatcagcaggcgggcggggagcaattgaacgcacttctgatgcggcgcgacttgtcgtaatatcccgccggtggcaaaacatccctgttttgctctccaagagcccgagaccatgcccgtactgaaatccccgcctgcgcacaagaataccggtatcgaggcccgctgcgccgccaccggcatgcgcatgaccgaacagcgccgcgtcatcgcgcgggtgctggcggagtccgtcgatcatcccgacgtcgaggaactgtaccggcgctgcgtcgcggtcgacgacaagatctcgatctcgaccgtgtaccgcacggtgaaactgttcgaggatgccggcatcatcgagcggcacgatttccgcgagggccgggcgcgctacgagcagatgcccgacagccaccacgaccatctgatcaatctgcgcgacggcaaggtgatcgagttcacctccgaggagatcgagaaactgcaggccgagatcgcccgcaagctcggctacaaactggtcgatcaccggctcgaattgtattgcgtgccgctggacgacgataagtcctgatagtcgccgtcattccggggcacgcgtctgcgcgtgaacccggaatctcgagattacgggttcgcttcgcgccccggaacgacaaggtaatagctggattttctgcgctttagcctataattgacgcgtcaaccgacgctggcgccgcattggcgccatgccaaccccgcattcaggttccatgaagccgccgcgcaagctgcacatcaagtcatatggctgccagatgaacgtctacgatgcgcagcgcatggtggacacgctggcgccggagggctttgtcgagaccgatcgcgccgaggatgcggacctggtgatcctcaacacctgtcacatccgcgagaaggcgtccgagaaggtctactccgaactcggccggctgcgtgtcgcgaaggatgaagccgcgcgccagggtcgcgagatgcgcatcgcggtcgccggctgcgtcgcgcaggccgagggccaagagatcatccgccgcgcgccgaccgtcgatgtcgtggtcggcccgcagagctatcatcatctgccgcagctattggcgcacgcgaaaagctatggccgcgcgctggagaccgaattcccggtcgaggacaagttcggattcctggcgccgccgaaaccggatgcgatccgcgcgcgcggcatctcttcgttcgtcaccgtgcaggaaggctgcgacaaattctgcaccttctgcgtagtcccttatacgcgcggcgccgaagtctcgcgcccggtgacaaagatcatcgacgacgtgatgcggctcgccgacaatggcgtgcgcgagatcactttgatcgggcagaacgtcaacgcctatcacggcgaaggccctgacggtgcgacatggccgctcggcaagctgctacatcggctcgccaagattcccggcatcgtgcggctgcgctattcgaccagccatccccgcgacgtcgacgattcactgatcgaggcgcatcgcgacctgcccgaactaatgccgttcgtgcaccttccggtgcaatccggttccgaccgggtactggccgccatgaaccggaagcataccgccgatgattaccgccgcgtcatcgaccgattccgtaccgcgcggcaagatattgcgttttcttcggattttatcgtcggcttccccggcgaaagcgagggagaatttgccgccaccctcgcgcttgtcatgcaaatcggatacgctggcgcctattcgttcaaatattcgccgcggccaggcacgccggcggcggacatgcgggagacggtgtcagcggcagcgatggacgagcgattggtgcggctccaggaattgatcgacagccagcaatcggccttcaacaaggccatgattggcaacaccgtcgatgtgctgttcgaacgcgcggcccgcaatcccggccagatcgtcggccgcaccgcctatctgcagccggcgcacgtgatggcgtcgccggacatcatcgggaaagtgctgccggtcaccatcgcaagcctcgagcgctacagcctgctcggcgaactcgccaggccagcggcgcggcccaccccatcaacgctttcaccaatgacgacgggagcctgaacccttgccaaaaagcgcatcggattcaccttcactcgctcccagccgcaaatttgaccgcgacatgcaaattccgcccgagacccaggtcgtcatcgacttcgacgacaaccgcgccgcttccgccctggtcggcccctacgggcaaaacctggcgcaaatcgaacgccggctcggcgtggtcgtggattcgcgcggcaaccacatcaccatcgccgggtcgcgcgacggctgcgacgccgcgcggcgcgtgctggagacgctctacgcccaggcggtgcaggggcacgatctggcgcagggcgacgtcgaaggcgcgatccgcgccgtgatcgcgcagggctcgctgttcgaattcgatcccaaggccgccaagaacgctttcgagaccatcaacctgcgcaagcgcccagtgcgggcgcgtactgccgcgcaggattcctacatccgcgcgttgaagcgccacgagatggtgttcggcatcggccccgccggcaccggcaagacctggcttgcggtggcgcatgccgcgcaattgttcgaacgcaaggaagtcgaccgcatcatcctgtcgcggcccgcggtcgaggccggcgaacgcctcggcttcctgcccggcgatttgcgcgaaaaggtcgatccctatctgcggccgatctacgacgcgctgtacgatctgatggacgcacgcgtcgtcgagcgcgcgctgcagggcggcgaaatcgagatcgcgccgctcgccttcatgcgcggccgcacgctgaccaatgccgtcatcatcctcgacgaggcgcaaaacaccacctcgatgcagatgaagatgttcctgacccggcttggcgagaacagccgcatgatcatcaccggcgatccctctcaggtcgacctgccgcatggccagacctcggggcttgccgaagccgtcaggctgctcgacggcgtcgagggcatctcgcaggtgaaattcaccgctgaggacgtcatccgccacgaactggtggctcggatcgtcgctgcctatgagggcttgccgcaaaagccggcaaccggcaaatcctgatggccagatcttcggccgcgggcctgccgatagcaggccgccgcggcaacccaatcgctggaatgcaaaacactggaacgaaattgtcttccgcccttccgctcaccgaagttctcgtcgtcgccgattgctggcaggcggagcccgatgccgaggcggtgatccatcgcgccgtccgtgcggcggccgagatcgctgattccgatgtcggcgaagccgaactcgcggtgatgctgaccgacgattccgggattcaaacgctgaaccgcaactggcgcggcatcgacaagccgaccaacgtgctgtcgtttccggcgttgcagccgacggggtcggcgacatccgacgatgtgccgcgcatgctcggcgatatcgccattgcctaccagacaacgcgcaaagaggccgacgacgaacagaagccgttcgaccatcacctcagccatctggcggtgcacggcttcctgcatctgatcggctacgatcacgagaaagacgacgacgccgaaaccatggaaagtctcgaacaggagattttggcgcagctcggcattcccgacccctatgcggaccgggtggactgacatgccggactccgatcccacccacgacaatccgcgcaatacgcgcaaccttcccgccgtggtgcaggacggcgaggtgctgcgcccggccgccgataactggctgacccgcgccatccgcacactgttcggctggaagccgggatcggtccgcgacgatttgcaggtcgtgctcgatgcgtcgacgccggacgaggtcggcttttccgccatcgagcgcaccatgctgcgcaatatcctcggcctgcacgaacgccgcatcgccgacgtcatgatccatcgcgccgacatcgtcgcggtcaagcgcgacatcccgctcggcgaattgatgagcctgttcgagagcgcggcgcattcgcgcctcgtcgtctacaacgagaccctcgacgatcccgaaggcatcgtgcacatccgcgacttgctggcgttcatgaccgcgaaggcacgagtcggcgagaccggcaaggccaagcgcaagaagccgttcccggcgggtcttgacctgcgcgccgtcgacctggcgctgccgctcagcgaggccaacatcatccgcaagctgctttacgtaccgccctcgatgcgggcgatcgacctgctggcgcagatgcaggcctcgcgcatccacctggcgctggtggtcgacgaatatggcggcaccgacgggctggtgtcgatcgaggacatcgtcgaacagatcgtcggcgaaatcgacgatgagcacgacagcgacgagccgccggcgatcgtccgccaggcggataattccttcatcgcggatgcgcgcgccagcctcgatgatgcccgcaccgtgatcggcgaggacttcgtcactggcgaagccggggaagaggtcgccacgcttggcggctatctggtgtcccatgtcggccgcctgccggtgcgcggcgaagtgatttcaggccccggcaattacgagatcgaggtgcttgacgccgatccgcgaagggtgaaacggctgcgcattgccgtccgcaaggagcgtcccgcagcccgcgcccagcgcgaaagccgccgccgcgaggccgcgcccgacagcaattcgccacaagccaatgacaatgcgccgccatcgtccagcgatggagccggctcgcagtgataccagccagcaaattccgcactgcgggtcttgcgaccatcctggcctggggatggaaacgcgctgctctcgcgctggtggccggcgcgttgtcggcgctcgcgatggcgccgatcaatgcatggccggtgctgttcctgacctttccggtcgcggtctggctgatcgatggcgccggtgccggacgctggcgcggcgtcccggcggcggcgatgtcgggcttctggttcgggctcggctatttcgtggccggactttactggatcggctacgccttcctggtcgatgcgccgacctttgcgtggctaatgccgtttgcggtggtgggcttgccggcctatctcgcgctgttcacggccgcaggctttgggctggcccgcctgctctggacccgcgatgcgtcgcgcctgctcgcgctcgcgatcagtctgacggtcaccgagtggctgcgcggccacctgctctccggatttccgtggaattcattcggctacgcgctgtccgagccgctggcactggcgcagacggcttcgctggtcgggctatggggcatgacgttcctcagcgtggcgatcttcgccagcccggctgtcctgatcgatggaagctcgcgcgggcgaaaaccctggctcgcgccggtggcggcgctggtactgctcgccgccatggccgtgttcggcgccgtccggctgtcgctgcagccgacgctgctcaccaaggtcaagctgcgcatcatgcagcccaatctgcagcaggacgtcaaattcaactacgccgccaaggcggaggtgatgcagaaatatctgacgctgtccgaccgcgcctccgggccgcaatcgaccggcgtgcgcgatacccagatcctgatctggccggaatcggcattcccgtttttcctcacccgcgaggccgatgcgatggcgcagatcgccgaattgctgcccaaggggacggtgctgatcacaggctcggtgcgcgcgcccgacctgccgcccggcgtccgcatcacgcgcgcctataactcgatctatgtgatcgaccatgacggaagcgtgctgcaggtctacgacaagctgcatctggtgccgttcggggagtatctgccgttccaggattggatggaaaaactcggcttcgtgcagctcacgcgcgtccagggtggattcattcccggcacgcgccgccgcaccatggagataccggacgctccgcgggcgctacctttgatttgctatgaggcaatttttcccggcgacattgccgcgcgcgacgatcgcccgggctggatcatcaacctgaccaatgacggctggtttggaatttcaaccggcccctaccagcatctgcagcaggcgcggctgcgcgcagtcgaggagggcctgcctgtcgttcgtgccgccaacaccggaatttcggcggtcatcgatccgtccggacgtattgtcgcacagctcggcctcggcattgaaggcgttttggacgccaacctgccccaggctttgccgccaacgatctacgcacgcatcaaggatattcccgcggccgtaatcatggccgccgcgttgctgctcgtcatcagacgccggtttgcaaggcgaaaggcctgaggtgtgggatccccgggtaccgagctcgaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagctgaatggcgaatggcgcctgatgcggtattttctccttacgcatctgtgcggtat

SEQ ID NO: 62 - DNA, 28 bp, Unknown
  Bradyrhizobium specie
ttcaaaattacggtgatattgcaatgat

SEQ ID NO: 63 - DNA, 32 bp, Unknown
  Bradyrhizobium specie
tatttaatccagatcggcgatcgattcaattt

SEQ ID NO: 64 - DNA, 29 bp, Unknown
  Bradyrhizobium specie
tatttaatccagatcggcgatcgattcaa

SEQ ID NO: 65 - DNA, 30 bp, Unknown
  Bradyrhizobium specie
tttaatccagatcggcgatcgattcaattt

SEQ ID NO: 66 - DNA, 28 bp, Unknown
  Bradyrhizobium specie
tttaatccagatcggcgatcgattcaat

SEQ ID NO: 67 - DNA, 29 bp, Unknown
  Bradyrhizobium specie
tttaatccagatcggcgatcgattcaatt

SEQ ID NO: 68 - DNA, 155 bp, Unknown
  Bradyrhizobium specie
attgtcgcaggatcaggcgcccggtttcgatcacggggccatcgacccgcatcagtctggcgcctggcttgagcgaagggatcatcggtttcctcatcctcaatacgtgtttggacgcgaccctgccattgttgctatgatcgctgcaaatccaa

SEQ ID NO: 69 - DNA, 30 bp, Unknown
  Bradyrhizobium specie
attgtcgcaggatcaggcgcccggtttcga

SEQ ID NO: 70 - DNA, 44 bp, Unknown
  Bradyrhizobium specie
tggacgcgaccctgccattgttgctatgatcgctgcaaatccaa

SEQ ID NO: 71 - DNA, 30 bp, Unknown
  Bradyrhizobium specie
tggacgcgaccctgccattgttgctatgat

SEQ ID NO: 72 - DNA, 28 bp, Unknown
  Bradyrhizobium specie
attgttgctatgatcgctgcaaatccaa

SEQ ID NO: 73 - DNA, 33 bp, Unknown
  Bradyrhizobium specie
atgaattctgttgtttccgaatgccacagtcgt

SEQ ID NO: 74 - DNA, 30 bp, Unknown
  Bradyrhizobium specie
atgaattctgttgtttccgaatgccacagt

SEQ ID NO: 75 - DNA, 29 bp, Unknown
  Bradyrhizobium specie
attctgttgtttccgaatgccacagtcgt

SEQ ID NO: 76 - DNA, 28 bp, Unknown
  Bradyrhizobium specie
tcgaatatctgtcgctgctcgcgattat

SEQ ID NO: 77 - DNA, 28 bp, Unknown
  Bradyrhizobium specie
tttatgtgctgaccgtcgagcaattcga

SEQ ID NO: 78 - DNA, 30 bp, Unknown
  Bradyrhizobium specie
tagacgcgccgcgcctcttcggcaaaaaat

SEQ ID NO: 79 - DNA, 29 bp, Unknown
  Bradyrhizobium specie
attttgagggtttgtcgcggtgtgttcag

SEQ ID NO: 80 - DNA, 28 bp, Unknown
  Bradyrhizobium specie
attttgagggtttgtcgcggtgtgttca

SEQ ID NO: 81 - DNA, 113 bp, Unknown
  Bradyrhizobium specie
tcgaaatcatcgcgttaccctcgcaatatcgaaagctacaaccattttcaccaattcgtgcagccggggaggcggcatttccgttgtaatctgaatgtcttactgtaaaaaat

SEQ ID NO: 82 - DNA, 29 bp, Unknown
  Bradyrhizobium specie
tcgaaatcatcgcgttaccctcgcaatat

SEQ ID NO: 83 - DNA, 30 bp, Unknown
  Bradyrhizobium specie
ttgtaatctgaatgtcttactgtaaaaaat

SEQ ID NO: 84 - DNA, 28 bp, Unknown
  Bradyrhizobium specie
ttgtaatctgaatgtcttactgtaaaaa

SEQ ID NO: 85 - DNA, 29 bp, Unknown
  Bradyrhizobium specie
tgtaatctgaatgtcttactgtaaaaaat

SEQ ID NO: 86 - DNA, 28 bp, Unknown
  Bradyrhizobium specie
tgtaatctgaatgtcttactgtaaaaaa

SEQ ID NO: 87 - DNA, 28 bp, Unknown
  Bradyrhizobium specie
aggaattcctctatcgccatcccaaaat

SEQ ID NO: 88 - DNA, 30 bp, Unknown
  Bradyrhizobium specie
tttgcaagtaatcttcgccctcaccaaaat

SEQ ID NO: 89 - DNA, 29 bp, Unknown
  Bradyrhizobium specie
ttgcaagtaatcttcgccctcaccaaaat

SEQ ID NO: 90 - DNA, 47 bp, Unknown
  Bradyrhizobium specie
tttttattttagggcttgacacgatttccgataaccggaagtaattt

SEQ ID NO: 91 - DNA, 30 bp, Unknown
  Bradyrhizobium specie
tttttattttagggcttgacacgatttccg

SEQ ID NO: 92 - DNA, 29 bp, Unknown
  Bradyrhizobium specie
ttgacacgatttccgataaccggaagtaa

SEQ ID NO: 93 - DNA, 30 bp, Unknown
  Bradyrhizobium specie
ttgacacgatttccgataaccggaagtaat

SEQ ID NO: 94 - DNA, 29 bp, Unknown
  Bradyrhizobium specie
tggaattgaaaggattgacccaccaaaaa

SEQ ID NO: 95 - DNA, 49 bp, Unknown
  Bradyrhizobium specie
ttcacaattcgtgatatatttatgatatcaaataatcgacgcgagtcaa

SEQ ID NO: 96 - DNA, 28 bp, Unknown
  Bradyrhizobium specie
ttcacaattcgtgatatatttatgatat

SEQ ID NO: 97 - DNA, 30 bp, Unknown
  Bradyrhizobium specie
ttatgatatcaaataatcgacgcgagtcaa

SEQ ID NO: 98 - DNA, 28 bp, Unknown
  Bradyrhizobium specie
atgatatcaaataatcgacgcgagtcaa

SEQ ID NO: 99 - DNA, 51 bp, Unknown
  Bradyrhizobium specie
agatttttgtcgtgctttcacactattcgagggaagttgccagattttcca

SEQ ID NO: 100 - DNA, 30 bp, Unknown
  Bradyrhizobium specie
agatttttgtcgtgctttcacactattcga

SEQ ID NO: 101 - DNA, 28 bp, Unknown
  Bradyrhizobium specie
atttttgtcgtgctttcacactattcga

SEQ ID NO: 102 - DNA, 30 bp, Unknown
  Bradyrhizobium specie
actattcgagggaagttgccagattttcca

SEQ ID NO: 103 - DNA, 29 bp, Unknown
  Bradyrhizobium specie
atgacgtattcgatctggggaccaatgat

SEQ ID NO: 104 - DNA, 30 bp, Unknown
  Bradyrhizobium specie
ttgattatctgcctcgtcggtttcgatgat

SEQ ID NO: 105 - DNA, 30 bp, Unknown
  Bradyrhizobium specie
attgtcgcgcgatatcgcgcagaagttcaa

SEQ ID NO: 106 - DNA, 29 bp, Unknown
  Bradyrhizobium specie
attgtcgcgcgatatcgcgcagaagttca

SEQ ID NO: 107 - DNA, 37 bp, Unknown
  Bradyrhizobium specie
tcgacgtcatcgacaaggacgtcgatatttcgatgct

SEQ ID NO: 108 - DNA, 29 bp, Unknown
  Bradyrhizobium specie
tcgacgtcatcgacaaggacgtcgatatt

SEQ ID NO: 109 - DNA, 28 bp, Unknown
  Bradyrhizobium specie
tcgacaaggacgtcgatatttcgatgct

SEQ ID NO: 110 - DNA, 30 bp, Unknown
  Bradyrhizobium specie
attgtatgaatgggccggattcggcgtcgt

SEQ ID NO: 111 - DNA, 28 bp, Unknown
  Bradyrhizobium specie
ctggattttctgcgctttagcctataat

SEQ ID NO: 112 - DNA, 97 bp, Unknown
  Bradyrhizobium specie
attctgcaccttctgcgtagtcccttatacgcgcggcgccgaagtctcgcgcccggtgacaaagatcatcgacgacgtgatgcggctcgccgacaat

SEQ ID NO: 113 - DNA, 29 bp, Unknown
  Bradyrhizobium specie
attctgcaccttctgcgtagtcccttata

SEQ ID NO: 114 - DNA, 29 bp, Unknown
  Bradyrhizobium specie
tcgacgacgtgatgcggctcgccgacaat

SEQ ID NO: 115 - DNA, 29 bp, Unknown
  Bradyrhizobium specie
tggaatgcaaaacactggaacgaaattgt

SEQ ID NO: 116 - DNA, 30 bp, Unknown
  Bradyrhizobium specie
attacaattcactggccgtcgttttacaac

SEQ ID NO: 117 - DNA, 28 bp, Unknown
  Bradyrhizobium specie
attacaattcactggccgtcgttttaca

SEQ ID NO: 118 - DNA, 19 bp, Unknown
  Bradyrhizobium specie
cactggccgtcgttttaca

SEQ ID NO: 119 - DNA, 8 bp, Unknown
  Bradyrhizobium specie
tttcaaaa

SEQ ID NO: 120 - DNA, 8 bp, Unknown
  Bradyrhizobium specie
ttttcacg

SEQ ID NO: 121 - DNA, 13 bp, Unknown
  Bradyrhizobium specie
tattttttatttt

SEQ ID NO: 122 - DNA, 8 bp, Unknown
  Bradyrhizobium specie
ctctccca

SEQ ID NO: 123 - DNA, 8 bp, Unknown
  Bradyrhizobium specie
ctctccca

SEQ ID NO: 124 - DNA, 8 bp, Unknown
  Bradyrhizobium specie
gaccccac

SEQ ID NO: 125 - DNA, 8 bp, Unknown
  Bradyrhizobium specie
gttcgagg

SEQ ID NO: 126 - DNA, 8 bp, Unknown
  Bradyrhizobium specie
ccggccta

SEQ ID NO: 127 - DNA, 8 bp, Unknown
  Bradyrhizobium specie
atttttcc

SEQ ID NO: 128 - DNA, 8 bp, Unknown
  Bradyrhizobium specie
cctatagt

SEQ ID NO: 129 - DNA, 70 bp, Unknown
  Bradyrhizobium specie
tttcaaaatcgggctggccgaaaccacaccggcctcaccgatttcaaaattacggtgatattgcaatgat

SEQ ID NO: 130 - DNA, 193 bp, Unknown
  Bradyrhizobium specie
atgaattctgttgtttccgaatgccacagtcgtctaccactgggatcggcggtgggggtaagcgcaggtggccagccaactattccatttgaaaccgggtatacgcccggcgcttagcgacatcatcggcggcagcgccgccagcgtcctcacggtgacattcggcctgtcatacgcgctgctgattttcacg

SEQ ID NO: 131 - DNA, 48 bp, Unknown
  Bradyrhizobium specie
tattttttattttagggcttgacacgatttccgataaccggaagtaat

SEQ ID NO: 132 - DNA, 582 bp, Unknown
  Bradyrhizobium specie
ctctcccatcgggagaggtcggcgcgcagcgccgggtgaggggttaggaaccatcgatagaccttatcccctcacccgctcgcaagatgctcgcgacctctcccgatgggagaggtaagaacactgcagttgcggcaagactcaagcaaacacaatagcgacgccgcttgtcgaaggcggctgcgccgtggcagcatgcgccggtttggcaagcaccgggacatgcatgaccacccagcgacgaagctacgagacgggtcacaatccaaaatgcctggtcatcgtcgacgacaccgcggagtgggaccgcgcggtctactatgccagccgctgggcgatgcgggtcggcggcggcgtcgtgatgctgcgcatcatcgaactcgaggaccagaaccagcaatggctgggggtcgccgacatcatgcgcgccgaggccgaggacgccgccaatgaggcgctcgatcgcgccgccggccgcgccaacggcattgccgcgatcacgccggagcgggtgatacgcgagggcgaccccaccgagcagatcctcgacgtcatcgacaaggacgtcgatatttcgatgct

SEQ ID NO: 133 - DNA, 359 bp, Unknown
  Bradyrhizobium specie
gttcgaggatgccggcatcatcgagcggcacgatttccgcgagggccgggcgcgctacgagcagatgcccgacagccaccacgaccatctgatcaatctgcgcgacggcaaggtgatcgagttcacctccgaggagatcgagaaactgcaggccgagatcgcccgcaagctcggctacaaactggtcgatcaccggctcgaattgtattgcgtgccgctggacgacgataagtcctgatagtcgccgtcattccggggcacgcgtctgcgcgtgaacccggaatctcgagattacgggttcgcttcgcgccccggaacgacaaggtaatagctggattttctgcgctttagcctataat

SEQ ID NO: 134 - DNA, 446 bp, Unknown
  Bradyrhizobium specie
atttttcccggcgacattgccgcgcgcgacgatcgcccgggctggatcatcaacctgaccaatgacggctggtttggaatttcaaccggcccctaccagcatctgcagcaggcgcggctgcgcgcagtcgaggagggcctgcctgtcgttcgtgccgccaacaccggaatttcggcggtcatcgatccgtccggacgtattgtcgcacagctcggcctcggcattgaaggcgttttggacgccaacctgccccaggctttgccgccaacgatctacgcacgcatcaaggatattcccgcggccgtaatcatggccgccgcgttgctgctcgtcatcagacgccggtttgcaaggcgaaaggcctgaggtgtgggatccccgggtaccgagctcgaattcgccctatagtgagtcgtattacaattcactggccgtcgttttacaac

SEQ ID NO: 135 - DNA, 1355 bp, Unknown
  Bradyrhizobium specie
ccggcctatctcgcgctgttcacggccgcaggctttgggctggcccgcctgctctggacccgcgatgcgtcgcgcctgctcgcgctcgcgatcagtctgacggtcaccgagtggctgcgcggccacctgctctccggatttccgtggaattcattcggctacgcgctgtccgagccgctggcactggcgcagacggcttcgctggtcgggctatggggcatgacgttcctcagcgtggcgatcttcgccagcccggctgtcctgatcgatggaagctcgcgcgggcgaaaaccctggctcgcgccggtggcggcgctggtactgctcgccgccatggccgtgttcggcgccgtccggctgtcgctgcagccgacgctgctcaccaaggtcaagctgcgcatcatgcagcccaatctgcagcaggacgtcaaattcaactacgccgccaaggcggaggtgatgcagaaatatctgacgctgtccgaccgcgcctccgggccgcaatcgaccggcgtgcgcgatacccagatcctgatctggccggaatcggcattcccgtttttcctcacccgcgaggccgatgcgatggcgcagatcgccgaattgctgcccaaggggacggtgctgatcacaggctcggtgcgcgcgcccgacctgccgcccggcgtccgcatcacgcgcgcctataactcgatctatgtgatcgaccatgacggaagcgtgctgcaggtctacgacaagctgcatctggtgccgttcggggagtatctgccgttccaggattggatggaaaaactcggcttcgtgcagctcacgcgcgtccagggtggattcattcccggcacgcgccgccgcaccatggagataccggacgctccgcgggcgctacctttgatttgctatgaggcaatttttcccggcgacattgccgcgcgcgacgatcgcccgggctggatcatcaacctgaccaatgacggctggtttggaatttcaaccggcccctaccagcatctgcagcaggcgcggctgcgcgcagtcgaggagggcctgcctgtcgttcgtgccgccaacaccggaatttcggcggtcatcgatccgtccggacgtattgtcgcacagctcggcctcggcattgaaggcgttttggacgccaacctgccccaggctttgccgccaacgatctacgcacgcatcaaggatattcccgcggccgtaatcatggccgccgcgttgctgctcgtcatcagacgccggtttgcaaggcgaaaggcctgaggtgtgggatccccgggtaccgagctcgaattcgccctatagtgagtcgtattacaattcactggccgtcgttttacaac

SEQ ID NO: 136 - DNA, 692 bp, Unknown
  Bradyrhizobium specie
atcctatacaagaaagacaagatgtacaacttgtgaacaataaaatacggccagccgcacgcaaccactgccagccgatccggtcctagctgtgggcgcggaaactaacggatcggagagacgggtccaaagctccgggctgttcccgagccgggcgcctgctagactcaaattccgcgcagactcacaagggatgcccgggaatccgaaaacaaccaaaaaggagtgcatccatgaaaggcaaatccttggcatggatctttaccgctgctctggcgggaaccggtctgagcggagtggtacaggcgcaagagaagttcgtcaccatcggcactggcggccagaccggcgtttattacgttgccgggcaatcgatctgccgcttcgtcaaccgcaatgcggaaaacatcaagtgcaacgccccggccagcggcggcggcgtagccaacgtcaacggcctgcgcagtggcgaattcaacttcggcatcatgcaatccgaccaccagtacaaggcaatggaaggcgtcgcccccttcgagaaagaagggaaaatggacgacatccgtgcagtgttctccctgcagagcgaggttttcaccgtactcgcccgccgcagcgccaacatcaagggcctcgacgacctcaagggcaagtgggatccccgggtaccgagctcgaattcgccctatagt

(19) United States
(12) Patent Application Publication
(10) Pub. No.: US 2019/0024193 A1
(43) Pub. Date: Jan. 24, 2019

(54) Sesquiterpene Biosensors and Uses Thereof

(71) Applicant: Prospect Bio, Inc., Brisbane, CA (US)

(72) Inventors: Cameron Strachan, Vienna (AT); Nina Patrick, San Francisco, CA (US)

(73) Assignee: Prospect Bio, Inc., Brisbane, CA (US)

(21) Appl. No.: 16/037,709

(22) Filed: Jul. 17, 2018

Related U.S. Application Data
(60) Provisional application No. 62/534,115, filed on Jul. 18, 2017.

(51) Int. Cl. C12Q 1/6897; C12N 15/70

(52) U.S. Cl. CPC C12Q 1/6897; C07C 33/14; C12N 15/70

(57) ABSTRACT

In an aspect, the invention relates to nucleic acids encoding new bisabolol responsive biosensor polypeptides useful for detecting responses to stimuli, identifying new enzymes, and novel pathways in cells. In other aspects, the invention relates to genes which induce expression in response to bisabolol and the gene products encoded by those genes. In still another aspect, the invention relates to control regions that induce expression in response to bisabolol. In an aspect, the invention also relates to the use of the bisabolol responsive biosensors, bisabolol responsive control regions, and the genes and encoded polypeptides that are expressed in response to bisabolol.

### CROSS-REFERENCES TO RELATED APPLICATIONS

This application claims priority to U.S. Provisional application Ser. No. 62/534,115 filed Jul. 18, 2017.

### REFERENCE TO SEQUENCE LISTING, TABLE OR COMPUTER PROGRAM

The official copy of the Sequence Listing is submitted concurrently with the specification as an ASCII formatted text file via EFS-Web, with a file name of “PB0014_ST25.txt”, a creation date of Jul. 18, 2017, and a size of 179 kilobytes. The Sequence Listing filed via EFS-Web is part of the specification and is incorporated in its entirety by reference herein.

### BACKGROUND OF THE INVENTION

Environmental microorganisms are an excellent source for making new enzymes, which can be used to engineer novel pathways into cells. However, environmental microorganisms can be difficult to culture in the laboratory let alone on an industrial scale. Accordingly, a number of metagenome screening methods have been developed to isolate useful genes from metagenomes, for example, metagenomic nucleotide sequencing methods (Okuta et al. Gene (1998) 212:221-228), and enzyme activity based screening (Henne et al. Appl. Environ. Microbiol. (1999) 65:3901-3907). Specific enzyme activity based screening methods for metagenome screening, include Substrate-Induced Gene-Expression (SIGEX) screening (Uchiyama et al. Nature Biotechnology (2005) 23(1):88-93) and more recently Product-Induced Gene-Expression (PIGEX) screening (Uchiyama and Miyazaki Appl. Environ. Microbiol. (2010) 76(21):7029-7035). Furthermore, several screening strategies have been developed to discover genetic elements that are activated in response to a metabolite, including intragenic genomic libraries and promoter traps (Uchiyama and Miyazaki PLOS ONE (2013) 8(9):e75795).

Sesquiterpenes are a class of terpenes which are hydrocarbons often found in the essential oils of plants. Sesquiterpenes may be acyclic or contain rings, and may include many combinations. Sesquiterpenes can be derived from isoprene units and many consist of three such isoprene units. Sesquiterpenes can be found naturally in plants and insects as defensive agents or pheromones.

One such sesquiterpene is bisabolol which includes (+)-α-bisabolol, (−)-α-bisabolol also known as levomenol, racemic α-bisabolol, (+)-β-bisabolol, (+-β-bisabolol, racemic β-bisabolol, and combinations of α-bisabolol and β-bisabolol. Bisabolol is a colorless viscous oil that is the primary constituent of the essential oil from German chamomile and Myoporum crassifolium. Bisabolol has a weak floral aroma and is used in various fragrances. It is also often used in cosmetics for its perceived skin healing properties. Bisabolol is known to have anti-irritant, anti-inflammatory, and anti-microbial properties.

### SUMMARY OF THE INVENTION

In an aspect, novel bisabolol biosensors are provided and uses of these biosensors are disclosed. Nucleic acids and polypeptides that encode the novel bisabolol biosensors of the invention are also disclosed. In other aspects, novel bisabolol control regions, and genes and gene products associated with the novel bisabolol control regions are disclosed. Nucleic acids can be comprised of a control region that responds to bisabolol operably linked to a nucleic acid encoding a reporter polypeptide and/or a selection marker. The nucleic acid encoding the reporter and the nucleic acid encoding the selection marker can be organized in an operon on the biosensor with each having a ribosome binding site. The reporter can be a moiety capable of being detected including, for example, a fluorescent reporter, a bioluminescent reporter, an X-ray reporter, a photoacoustic reporter, and/or an ultrasound reporter. The selection marker can encode a polypeptide that confers a trait upon the host cell that is suitable for artificial selection. Examples of selection markers include nucleic acids encoding polypeptides that confer an antibiotic resistance to the host cell, nucleic acids encoding a polypeptide that complements an auxotrophic mutation in the host cell, or a polynucleotide that encodes a polypeptide that detoxifies a molecule. The ribosome binding site can be compatible with the host cell and can initiate translation at a desired rate. The biosensors can also be transcribed from a promoter that is compatible with the host cell and initiates transcription at a desired rate.

In an aspect, the invention relates to control regions, genes, and gene products that respond to or are made in response to bisabolol. The genes whose expression is induced by bisabolol and gene products that are made in response to bisabolol may originate from Pseudomonas and include, for example, a sensor domain-containing diguanylate cyclase (SEQ ID NO: 1), a PAS domain-containing protein (SEQ ID NO: 2 or 4), a CadC family transcriptional regulator (SEQ ID NO: 3), SEQ ID NO: 5, SEQ ID NO: 6, and a C4-dicarboxylate ABC transporter substrate-binding protein (SEQ ID NO: 7). The genes whose expression is induced by bisabolol and gene products made in response to bisabolol can be a sensor domain-containing diguanylate cyclase (SEQ ID NO: 1), and a PAS domain-containing protein (SEQ ID NO: 2). The gene whose expression is induced by bisabolol and gene product made in response to bisabolol can be a sensor domain-containing diguanylate cyclase (SEQ ID NO: 1).

The genes whose expression is induced by bisabolol and gene products that are made in response to bisabolol may also originate from Bradyrhizobium and include, for example, a serine hydrolase (SEQ ID NO: 8), SEQ ID NO: 9, SEQ ID NO: 10, a N-acetyltransferase (SEQ ID NO: 11), a glycoside-hydrolase-family-3-domain-protein-K05349-beta-glucosidase (SEQ ID NO: 12), a phosphoribosylaminoimidazole-succinocarboxamide synthase (SEQ ID NO: 13), a enoyl-CoA hydratase (SEQ ID NO: 14), a sulfate permease from the SulP family (SEQ ID NO: 15), a succinate semialdehyde dehydrogenase (SEQ ID NO: 16), SEQ ID NO: 17, a 1,4-butanediol diacrylate esterase (SEQ ID NO: 18), an AMP-binding protein or fatty-acyl-CoA synthase (SEQ ID NO: 19), SEQ ID NO: 20, an Arc family DNA binding domain-containing protein (SEQ ID NO: 21), a regulator of protease activity HflC from the stomatin/prohibitin superfamily (SEQ ID NO: 22), an adenosine or carbohydrate kinase (SEQ ID NO: 23), a signal transduction histidine kinase (SEQ ID NO: 24), a murein biosynthesis integral membrane protein MurJ (SEQ ID NO: 25), a tryptophanyl-tRNA synthetase (SEQ ID NO: 26), an universal stress protein UspA (SEQ ID NO: 27), an Fe—S cluster biogenesis protein NfuA, 4Fe-4S-binding domain (SEQ ID NO: 28), an adenosine(37)-N6)-threonylcarbamoyltransferase complex dimerization subunit type 1 TsaB (SEQ ID NO: 29), a ribosomal-protein-alanine N-acetyltransferase (SEQ ID NO: 30), a transcriptional repressor, ferric uptake regulator, Fur family (SEQ ID NO: 31), a N6-isopentenyl adenosine(37)-C2)-methylthiotransferase MiaB (SEQ ID NO: 32), a phosphate starvation-inducible protein PhoH (SEQ ID NO: 33), a rRNA maturation RNase YbeY (SEQ ID NO: 34), a CBS domain containing DNA-binding protein (SEQ ID NO: 35), and an apolipoprotein N-acyltransferase (SEQ ID NO: 36). The genes whose expression is induced by bisabolol and gene products made in response to bisabolol can be a 1,4-butanediol diacrylate esterase (SEQ ID NO: 18), an AMP-binding protein or fatty-acyl-CoA synthase (SEQ ID NO: 19), SEQ ID NO: 20, an Arc family DNA binding domain-containing protein (SEQ ID NO: 21), a regulator of protease activity HflC from the stomatin/prohibitin superfamily (SEQ ID NO: 22). The genes whose expression is induced by bisabolol and gene products made in response to bisabolol can be a 1,4-butanediol diacrylate esterase (SEQ ID NO: 18) and an AMP-binding protein or fatty-acyl-CoA synthase (SEQ ID NO: 19). The gene whose expression is induced by bisabolol and gene product made in response to bisabolol can be an AMP-binding protein or fatty-acyl-CoA synthase (SEQ ID NO: 19). The genes whose expression is induced by bisabolol and gene products made in response to bisabolol can be SEQ ID NO: 20, an Arc family DNA binding domain-containing protein (SEQ ID NO: 21), and a regulator of protease activity HflC from the stomatin/prohibitin superfamily (SEQ ID NO: 22). The gene whose expression is induced by bisabolol and gene product made in response to bisabolol can be SEQ ID NO: 20.

A nucleic acid of the invention may encode the polypeptide of any one or more of SEQ ID NOs: 1-36. Nucleic acids may include those that hybridize under stringent hybridization conditions to a nucleic acid encoding one or more of SEQ ID NOs: 1-36. The nucleic acids may encode a polypeptide that has 70%, 80%, 90%, 95% or 99% sequence identity with one of SEQ ID NOs: 1-36. The nucleic acids may encode one of SEQ ID NOs: 1-36, hybridize under stringent hybridization conditions with a nucleic acid encoding one of SEQ ID NOs: 1-36, or encode a polypeptide that has 70%, 80%, 90%, 95% or 99% sequence identity with one of SEQ ID NOs: 1-36. A nucleic acid may encode the polypeptide of one of SEQ ID NOs: 1-2 or 18-22. Nucleic acids may include those that hybridize under stringent hybridization conditions to a nucleic acid encoding one or more of SEQ ID NOs: 1-2 or 18-22. The nucleic acids may encode a polypeptide that has 70%, 80%, 90%, 95% or 99% sequence identity with one of SEQ ID NOs: 1-2 or 18-22. The nucleic acids may encode one of SEQ ID NOs: 1-2 or 18-22, hybridize under stringent hybridization conditions with a nucleic acid encoding one of SEQ ID NOs: 1-2 or 18-22, or encode a polypeptide that has 70%, 80%, 90%, 95% or 99% sequence identity with one of SEQ ID NOs: 1-2 or 18-22.

Polypeptides may include polypeptides that have 70%, 80%, 90%, 95% or 99% sequence identity with one of SEQ ID NOs: 1-36. Polypeptides may include polypeptides encoded by nucleic acids that hybridize under stringent hybridization conditions with a nucleic acid encoding one of SEQ ID NOs: 1-36. The polypeptides may include one of SEQ ID NOs: 1-36, polypeptides encoded by nucleic acids that hybridize under stringent hybridization conditions with a nucleic acid encoding one of SEQ ID NOs: 1-36, or polypeptides that have 70%, 80%, 90%, 95% or 99% sequence identity with one of SEQ ID NOs: 1-36. Polypeptides may include polypeptides that have 70%, 80%, 90%, 95% or 99% sequence identity with one of SEQ ID NOs: 1-2 or 18-22. Polypeptides may include polypeptides encoded by nucleic acids that hybridize under stringent hybridization conditions with a nucleic acid encoding one of SEQ ID NOs: 1-2 or 18-22. The polypeptides may include one of SEQ ID NOs: 1-2 or 18-22, polypeptides encoded by nucleic acids that hybridize under stringent hybridization conditions with a nucleic acid encoding one of SEQ ID NOs: 1-2 or 18-22, or polypeptides that have 70%, 80%, 90%, 95% or 99% sequence identity with one of SEQ ID NOs: 1-2 or 18-22.

The bisabolol responsive control region can include at least one of SEQ ID NOs: 41-50 and 136 from Pseudomonas, SEQ ID NOs: 62-135 from Bradyrhizobium, and/or SEQ ID NOs: 51-60. The bisabolol responsive control region can include one or more of SEQ ID NOs: 41-47 and 136 from Pseudomonas and can include at least one of the promoters of SEQ ID NOs: 43, 45-47. The bisabolol responsive control region can include one or more of SEQ ID NOs: 99-102 from Bradyrhizobium and can include one or more of the promoters at SEQ ID NOs: 100-102. The bisabolol responsive control region can include at least one of SEQ ID NOs: 41-60, and 62-136 and can include 1000 base pairs 5′ and 3′ of the sequence in the SEQ ID NO. The bisabolol responsive control region can be at least one of SEQ ID NOs: 41-60, and 62-136 and can include 500 base pairs 5′ and 3′ of the sequence in the SEQ ID NO. The bisabolol responsive control region can be at least one of SEQ ID NOs: 41-60, and 62-136 and can include 100 base pairs 5′ and 3′ of the sequence in the SEQ ID NO. The bisabolol responsive control regions can include those nucleic acids that hybridize under stringent hybridization conditions with one or more of SEQ ID NOs: 41-60, and 62-136. The bisabolol responsive nucleic acids can include control elements in the RNA transcript which are encoded in SEQ ID NO: 37.

The bisabolol responsive control regions and/or biosensors can respond to a range of bisabolol from 50 μM to 2 mM. The bisabolol responsive control regions and/or biosensors can respond up to the solubility limit of bisabolol or to the toxicity limit of bisabolol. The bisabolol responsive control regions and/or biosensors can have a 3-100 fold change in expression in response to bisabolol (expression rate with no bisabolol to max expression rate with bisabolol). The bisabolol responsive control regions and/or biosensors can have a 3-10 fold change in expression in response to bisabolol. The bisabolol responsive control regions and/or biosensors can detect at least 50 μM bisabolol. Host cells with the bisabolol responsive control regions and/or biosensors can have a coefficient of variation of about 1-5%. Host cells with the bisabolol responsive control regions and/or biosensors can have a coefficient of variation of about 2%.

Host cells can contain the nucleic acids and/or polypeptides described herein. The host cells can be prokaryotic cells. The prokaryotic cell can be a species from Acidovorax, Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. The host cell can be E. coli. The host cell can be a eukaryotic cell. The eukaryotic cell can be an algae specie and/or a photosynthetic microorganism from Agmenellum, Amphora, Anabaena, Ankistrodesmus, Botryococcus, Boekelovia, Borodinella, Carteria, Chaetoceros, Chlamydomonas, Chlorella, Chlorococcum, Chlorogonium, Chrysosphaera, Cricosphaera, Cryptomonas, Cyclotella, Dunaliella, Ellipsoidon, Eremosphaera, Euglena, Fragilaria, Gleocapsa, Gloeothamnion, Hymenomonas, Isochrysis, Lepocinclis, Monoraphidium, Nannochloris, Nannochloropsis, Navicula, Nephrochloris, Nitschia, Nitzschia, Ochromonas, Oocystis, Oscillatoria, Pascheria, Phagus, Phormidium, Platymonas, Pleurochrysis Prototheca, Pyrobotrys Scenedesmus, Spirogyra, Tetraedron, Tetraselmis, or Volvox. The host cell can be Botryococcus braunii, Prototheca krugani, Prototheca moriformis, Prototheca portoricensis, Prototheca stagnora, Prototheca wickerhamii, or Prototheca zopfii. The eukaryotic cell can be a fungi specie from Aspergillus, Candida, Chlamydomonas, Chrysosporium, Cryotococcus, Debaromyces, Fusarium, Hansenula, Kluyveromyces, Neotyphodium, Neurospora, Penicillium, Pichia, Saccharomyces, Schizosaccharomyce, Trichoderma, Xanthophyllomyces, Yarrowia, and Zygosaccharomyces. The fungi can be one of Pichia pastoris, Saccharomyces cerevisiae, Schizosaccharomyces pompe, Kluyveromyces lactic, Hansenula polymorpha, or a filamentous fungi, e.g. Trichoderma, Aspergillus sp., including Aspergillus niger, Aspergillus phoenicis, Aspergillus carbonarius.

The bisabolol biosensors can be used in a host cell to monitor bisabolol in an environment, e.g., in the host cell, in a media containing the host cell, or in a subject containing the host cell. The bisabolol biosensors can be used to monitor metabolism involving bisabolol, discover genes, enzymes and other polypeptides involved in metabolism involving bisabolol, and/or monitor recombinant expression of polypeptides involved in metabolism involving bisabolol. The bisabolol biosensor can be used to select for host cells that can make or transport into the cell sufficient bisabolol to induce the biosensor to express protective amounts of the selection marker. The host cells can be grown in the presence of a selective agent so that cells expressing the selection marker can grow and cells that do not induce expression of the selection marker die or grow poorly. The bisabolol can be added to or present in a media and/or complex mixture (e.g., food, water, soil, waste, plant, or air/gas samples or extracts).

The bisabolol responsive control regions and/or biosensors can be used to discover host cells that make bisabolol. The bisabolol biosensors can be used in high-throughput screening to detect production of bisabolol from a host cell. Nucleic acids encoding the bisabolol responsive control regions can be operably linked to a nucleic acid encoding a reporter, and/or a bisabolol biosensor is placed into a plurality of host cells. A plurality of host cells can be different host cells. The plurality of host cells can be the same host cell that is subjected to different conditions. The plurality of host cells can be tested individually (as individual clones). The plurality of host cells can be tested in a multiplex fashion where multiple clones are grouped together and tested for activity. The host cells in the positive wells (or tubes/containers) can then tested on an individual cell basis or subjected to other testing to de-convolute the mixture of cells and identify the positive clone(s). The bisabolol responsive control regions and/or biosensors can be used to screen 10,000 to 1,000 clones per day. The bisabolol responsive control regions and/or biosensors can be used to screen hundreds of millions of clones per day, 1,000,000 to 100 clones per day, or 1,000 to 100 clones per day.

The bisabolol responsive control regions can be used to express polypeptides in a host cell in response to bisabolol. The polypeptide expressed in the host cell can be a heterologous polypeptide. The polypeptide expressed in the host cell can be one that can be used in a metabolic pathway that includes bisabolol. The bisabolol responsive control region can be SEQ ID NO: 37, SEQ ID NO: 44, or SEQ ID NO: 62-69.

### BRIEF DESCRIPTION OF THE DRAWINGS

FIG. 1 shows an exemplary biosensor construct.

FIG. 2 shows the response of a PB2 bisabolol biosensor to (−)-α-bisabolol when the construct with the PB2 biosensor has a high copy number.

FIG. 3 shows the response of a PB2 bisabolol biosensor to (−)-α-bisabolol when the construct with the PB2 biosensor has a low copy number.

FIG. 4 shows the response of a PB3 bisabolol biosensor to (−)-α-bisabolol when the construct with the PB3 biosensor has a high copy number.

FIG. 5 shows the response of a PB3 bisabolol biosensor to (−)-α-bisabolol when the construct with the PB3 biosensor has a low copy number.

FIG. 6 shows the response of a PB2 bisabolol biosensor to (−)-α-bisabolol when the construct with the PB2 biosensor has a low copy number and is grown with 200 μg/ml spectinomycin.

FIG. 7 shows the response of a PB3 bisabolol biosensor to (−)-α-bisabolol when the construct with the PB3 biosensor has a low copy number and is grown with 500 μg/ml spectinomycin.

FIG. 8 shows the response of a PB2 bisabolol biosensor to stimulation with (−)-α-bisabolol, squalene, nerolidol, cintonellal, linalool, carvone, and cineole.

FIG. 9 shows the response of a PB3 bisabolol biosensor to stimulation with (−)-α-bisabolol, squalene, nerolidol, cintonellal, linalool, carvone, and cineole.

FIG. 10 shows the DNA sequence for the biosensor insert. Underlined and italicized sequences are the mobile genetic element (ME), bold underlined sequences are the shine-dalgarno sequences, and underlined sequences are the open reading frames for the Gemini reporter and Spectinomycin resistance.

FIG. 11 shows a MFE structure for PB2 upstream (SEQ ID NO: 37).

FIG. 12 shows a centroid structure for PB2 upstream (SEQ ID NO: 37).

### DETAILED DESCRIPTION OF THE INVENTION

Before the various embodiments are described, it is to be understood that the teachings of this disclosure are not limited to the particular embodiments described, and as such can vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Numerical limitations given with respect to concentrations or levels of a substance are intended to be approximate, unless the context clearly dictates otherwise. Thus, where a concentration is indicated to be (for example) 10 it is intended that the concentration be understood to be at least approximately or about 10 μg.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings.

As used herein, “codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome.

As used herein, “consensus sequence” and “canonical sequence” refer to an archetypical amino acid sequence against which all variants of a particular protein or sequence of interest are compared. The terms also refer to a sequence that sets forth the nucleotides that are most often present in a DNA sequence of interest among members of related gene sequences. For each position of a gene, the consensus sequence gives the amino acid that is most abundant in that position in a multiple sequence alignment (MSA).

As used herein, “control sequence” refers to components, which are used for the expression of a polynucleotide and/or polypeptide of the present invention. Each control sequence may be native or foreign to the nucleic acid sequence encoding the polypeptide. Such control sequences may include, but are not limited to, some or all of the following: a promoter, an enhancer, an operator, an attenuator, a ribosome binding site (e.g., shine-dalgarno sequence), a leader, a polyadenylation sequence, a pro-peptide sequence, a signal peptide sequence, and a transcription terminator. At a minimum, the control sequences include a promoter and transcriptional signals, and where appropriate, translational start and stop signals.

As used herein, an “effective amount” refers to an amount of a compound, formulation, material, or composition, as described herein effective to achieve a particular biological result.

As used herein, the terms “expression vector” or “expression construct” or “recombinant DNA construct” refer to a nucleic acid construct, that has been generated recombinantly or synthetically via human intervention, including by recombinant means or direct chemical synthesis, with a series of specified nucleic acid elements that permit transcription and/or translation of a particular nucleic acid in a host cell. The expression vector can be part of a plasmid, virus, or nucleic acid fragment. Typically, the expression vector includes a nucleic acid to be transcribed operably linked to a promoter. The expression vector can exist in a host cell as either an episomal or integrated vector/construct.

As used herein, “exogenous gene” refers to a nucleic acid that codes for the expression of an RNA and/or protein that has been introduced (“transformed”) into a cell. A transformed cell may be referred to as a recombinant cell, into which additional exogenous gene(s) may be introduced. The exogenous gene may be from a different species (and so heterologous), or from the same species (and so homologous), relative to the cell being transformed. Thus, an exogenous gene can include a homologous gene that occupies a different location in the genome of the cell or is under different control, relative to the endogenous copy of the gene. An exogenous gene may be present in more than one copy in the cell. An exogenous gene may be maintained in a cell as an insertion into the genome or as an episomal molecule.

As used herein, “heterologous” polynucleotide or polypeptide refers to any polynucleotide that is introduced into a host cell by laboratory techniques, or a polynucleotide that is foreign to a host cell. As such, the term includes polynucleotides that are removed from a host cell, subjected to laboratory manipulation, and then reintroduced into a host cell. The introduced polynucleotide can express a heterologous polypeptide. Heterologous polypeptides are those polypeptides that are foreign to the host cell being utilized.

As used herein, “isolated polypeptide” refers to a polypeptide which is substantially separated from other components that naturally accompany it, e.g., protein, lipids, and polynucleotides. The term embraces polypeptides which have been removed or purified from their naturally-occurring environment or expression system (e.g., host cell or in vitro synthesis). The polypeptides of the invention may be present within a cell, present in the cellular medium, or prepared in various forms, such as lysates or isolated preparations.

As used herein, “naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

As used herein, “operably linked” and “operable linkage” refer to a configuration in which a control sequence or other nucleic acid is appropriately placed (i.e., in a functional relationship) at a position relative to a polynucleotide of interest such that the control sequence or other nucleic acid can interact with the polynucleotide of interest. In the case of a control sequence, operable linkage means the control sequence directs or regulates the expression of the polynucleotide and/or polypeptide of interest. In the case of polypeptides, operably linked refers to a configuration in which a polypeptide is appropriately placed at a position relative to a polypeptide of interest such that the polypeptide can interact as desired with the polypeptide of interest.

As used herein, “percentage of sequence identity” and “percentage homology” are used interchangeably herein to define to comparisons among polynucleotides or polypeptides, and are determined by comparing two optimally aligned sequences over a comparison window, where the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, Adv Appl Math. 2:482, 1981; by the homology alignment algorithm of Needleman and Wunsch, J Mol Biol. 48:443, 1970; by the search for similarity method of Pearson and Lipman, Proc Natl Acad Sci. USA 85:2444, 1988; by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Greene Publishing Associates, Inc. and John Wiley & Sons, Inc., (1995 Supplement). Examples of algorithms that are suitable for determining percent sequence identity and sequence similarity are the BLAST and BLAST 2.0 algorithms, which are described in Altschul et al., J. Mol. Biol. 215:403-410, 1990; and Altschul et al., Nucleic Acids Res. 25(17):3389-3402, 1977; respectively. Software for performing BLAST analyses is publicly available through the National Center for Biotechnology Information website. BLAST for nucleotide sequences can use the BLASTN program with default parameters, e.g., a word length (W) of 11, an expectation (E) of 10, M=5, N=−4, and a comparison of both strands. BLAST for amino acid sequences can use the BLASTP program with default parameters, e.g., a word length (W) of 3, an expectation (E) of 10, and the BLOSUM62 scoring matrix (see Henikoff and Henikoff, Proc Natl Acad Sci. USA 89:10915, 1989). Exemplary determination of sequence alignment and % sequence identity can also employ the BESTFIT or GAP programs in the GCG Wisconsin Software package (Accelrys, Madison Wis.), using default parameters provided.

As used herein, “recombinant” or “engineered” or “non-naturally occurring” refers to a cell, nucleic acid, protein or vector that has been modified due to the introduction of an exogenous nucleic acid or the alteration of a native nucleic acid. Thus, e.g., recombinant cells express genes that are not found within the native (non-recombinant) form of the cell or express native genes differently than those genes are expressed by a non-recombinant cell. A “recombinant nucleic acid” is a nucleic acid made, in general, by the manipulation of nucleic acid, e.g., using polymerases and endonucleases, or otherwise into a form not normally found in nature. Recombinant nucleic acids may be produced, for example, to place two or more nucleic acids in operable linkage. Thus, an isolated nucleic acid or an expression vector formed in vitro by ligating DNA molecules that are not normally joined in nature, are both considered recombinant for the purposes of this invention. Once a recombinant nucleic acid is made and introduced into a host cell or organism, it may replicate using the in vivo cellular machinery of the host cell; however, such nucleic acids, once produced recombinantly, although subsequently replicated intracellularly, are still considered recombinant for purposes of this invention. Similarly, a “recombinant protein” is a protein made using recombinant techniques, i.e., through the expression of a recombinant nucleic acid.

As used herein, “reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. A “reference sequence” can be based on a primary amino acid sequence, where the reference sequence is a sequence that can have one or more changes to the primary sequence.

As used herein, the term “reporter gene” refers to a polynucleotide that encodes a reporter molecule that can be detected, either directly or indirectly. Exemplary reporter genes encode, among others, enzymes, fluorescent proteins, bioluminescent proteins, receptors, antigenic epitopes, and transporters.

As used herein, a “ribosome binding site” refers to a sequence of nucleotides upstream of the start codon of an mRNA transcript that is responsible for the recruitment of a ribosome during the initiation of protein translation.

As used herein, a “selection marker” refers to a gene introduced into a host cell that confers upon the host cell a trait suitable for artificial selection.

As used herein, “stringent hybridization conditions” refers to hybridizing in 50% formamide at 5×SSC at a temperature of 42° C. and washing the filters in 0.2×SSC at 60° C. (1×SSC is 0.15 M NaCl, 0.015 M sodium citrate.) Stringent hybridization conditions also encompasses low ionic strength and high temperature for washing, for example 0.015 M sodium chloride/0.0015 M sodium citrate/O. 1% sodium dodecyl sulfate at 50° C.; hybridization with a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

As used herein, “substantial identity” refers to a polynucleotide or polypeptide sequence that has at least 80 percent sequence identity, at least 85 percent identity and 89 to 95 percent sequence identity. Substantial identity also encompasses at least 99 percent sequence identity as compared to a reference sequence over a comparison window of at least 20 residue positions or a window of at least 30-50 residues, wherein the percentage of sequence identity is calculated by comparing the reference sequence to a sequence that includes deletions or additions or substitutions over the window of comparison. Applied to polypeptides, the term “substantial identity” means that two polypeptide sequences, when optimally aligned, such as by the programs GAP or BESTFIT using standard parameters, i.e., default parameters, share at least 80 percent sequence identity, preferably at least 89 percent sequence identity, at least 95 percent sequence identity or more (e.g., 99 percent sequence identity).

As used herein, “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure polypeptide composition will comprise about 60% or more, about 70% or more, about 80% or more, about 90% or more, about 95% or more, and about 98% or more of all macromolecular species by mole or % weight present in the composition. The object species can be purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species.

### Biosensors

Biosensors can be encoded by nucleic acids comprised of a control region that responds to bisabolol which control region is operably linked to polynucleotides encoding a reporter polypeptide and/or a selection marker. The reporter is a polypeptide capable of being detected which includes, for example, a fluorescent reporter, a bioluminescent reporter, an enzyme, an X-ray reporter, a photoacoustic reporter, and an ultrasound reporter. The selection marker can encode a polypeptide that confers a trait upon the host cell that is suitable for artificial selection. Examples of selection markers include nucleic acids encoding polypeptides that confer an antibiotic resistance to the host cell, nucleic acids encoding a polypeptide that complements an auxotrophic mutation (e.g., auxotrophic mutations can inhibit the cell's ability to make an amino acid) in the host cell, or nucleic acids encoding a polypeptide that can detoxify a molecule. The antibiotic resistance can be encoded by any of the resistance genes known in the art including, for example, those described in van Hoek et al, Front. Microbiol. 2:1-27 (September 2011), which is incorporated by reference in its entirety for all purposes. The ribosome binding site(s) of the biosensor are compatible with the host cell and have a high translation initiation rate in the host cell (e.g., a strong shine dalgarno sequence for E. coli). The ribosome binding site can have a low translation initiation rate in the host cell. The biosensor can be made with a panel of ribosome binding sites of different strength to provide different windows of detection for the stimulus.

The polynucleotide encoding the reporter and the polynucleotide encoding the selectable marker can be organized in an operon so that both are expressed from the control region in the metagenomic nucleic acids. The selection marker can be expressed at low levels from the control region to produce a detectable phenotype. The selection marker can give the host cell resistance to an antibiotic. Low, constitutive expression from the control region in the metagenomic nucleic acids can express enough antibiotic resistance polypeptide so that the host cell can withstand a low amount of antibiotic in the growth media. The ribosome binding site for the selection marker can be strong and can provide a high level of translation initiation of the selection marker. The polynucleotide encoding the reporter can produce polypeptide that can be detected (directly or indirectly) when the bisabolol responsive control region expresses the reporter at a higher rate than the low constitutive level. The control region can be induced to increase transcription and this higher level of transcription can produce enough reporter polypeptide for detection.

The ribosome binding site of the biosensor can be a ribosome binding site such as ACAGGAAAG (SEQ ID NO: 3), TAAGGAGGT (SEQ ID NO: 4), or many other ribosome binding sites that are well known in the art. The strength or amount of translation initiated from the ribosome binding site can be altered by changing the ribosome binding site in ways known in the art. The reporter can be a Gemini reporter made of a fusion between a C-terminal portion of the alpha fragment of β-galactosidase and the N-terminus of full length green fluorescent protein, as described in Marin et al, PLoS ONE 4:e7569 (2009), which is incorporated by reference in its entirety for all purposes. The GFP portion of the Gemini reporter can be optically assayed to measure expression. The alpha fragment of β-galactosidase of the Gemini reporter can have enzymatic activity which can be complemented by the β-galactosidase omega fragment to produce enzymatic activity. The enzymatic activity of β-galactosidase can be more sensitive for low expression than the fluorescence from GFP because the enzyme activity amplifies the expression signal. The reporter can also be any other reporter gene described herein and/or known in the art.

The selection marker can be a Spectinomycin resistance gene as described in Clark et al., Antimicrob. Agents Chemotherap. 43:157-160 (1999), which is incorporated by reference in its entirety for all purposes. The selection marker can also be any other selection gene described herein and/or known in the art. The biosensor construct can have a nucleic acid encoding a Gemini reporter (reporter gene) and can have a nucleic acid encoding Spectinomycin resistance (selection gene) organized in an operon so that both nucleic acids can be expressed when the bisabolol control region is transcribed.

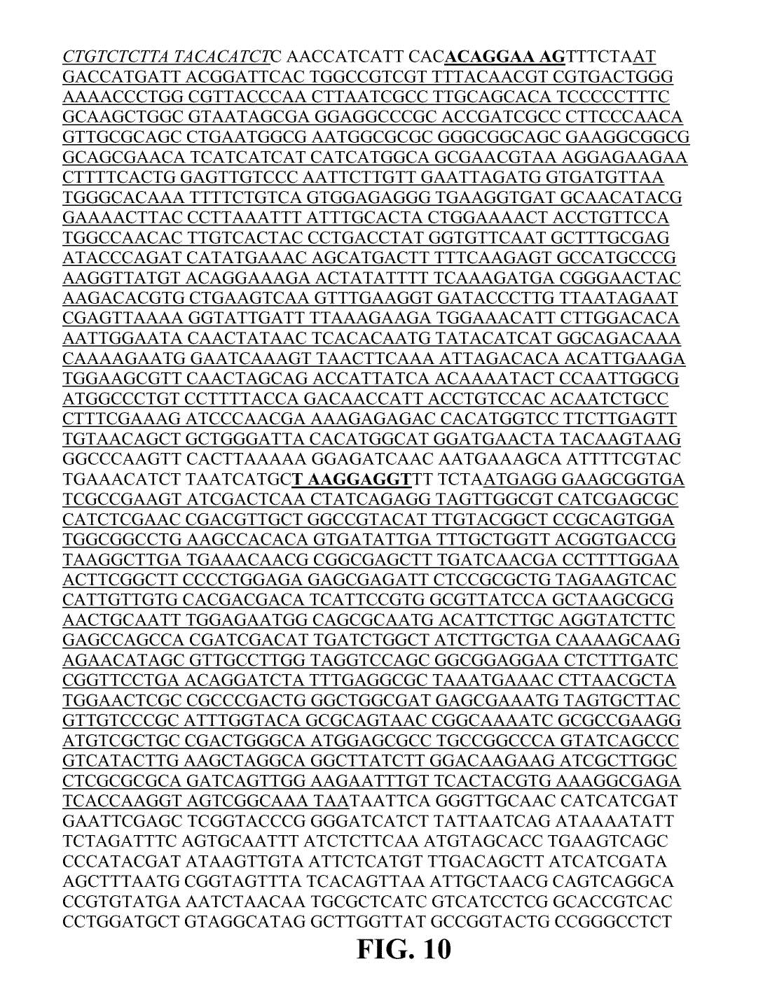

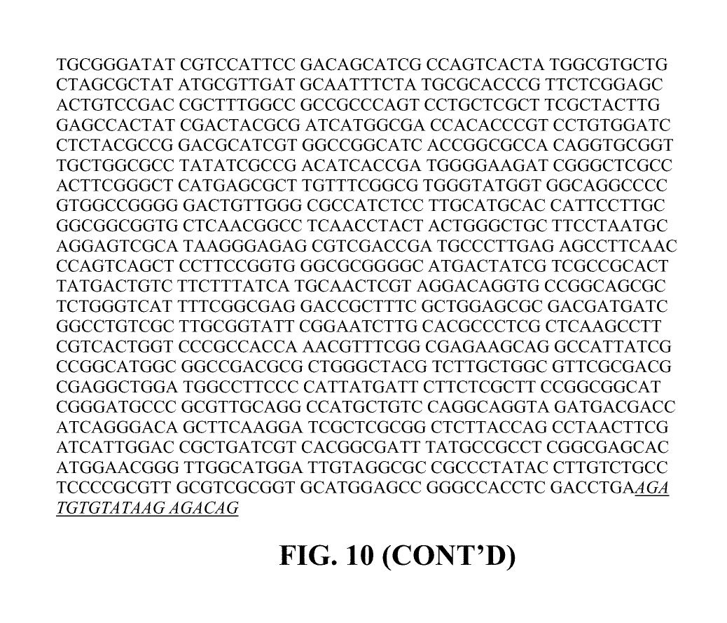

In an aspect of the invention, the bisabolol responsive control regions can be one of SEQ ID NOs: 37-44 and 46-89, and FIG. 10 shows the sequence for the Gemini reporter is (encoded at sequences 49-999), the ribosome binding site for the Gemini reporter is at sequences 34-42, the Spectinomycin resistance (encoded at sequences 1085-1873), and the ribosome binding site for the Spectinomycin resistance is at sequences 1072-1077 (SEQ ID NO: 91). These sequences are underlined (coding sequences) and bold underlined (ribosome binding sites) in FIG. 10.

The bisabolol responsive control regions and/or biosensors can have a limit of detection of about 50 μM for (−)-α-bisabolol. The bisabolol responsive control regions and/or biosensors can have a dynamic range of detection for (−)-α-bisabolol of about 50 μM to 2 mM. The bisabolol responsive control regions and/or biosensors can detect (−)-α-bisabolol up to the saturation point of (−)-α-bisabolol in solution. The bisabolol responsive control regions and/or biosensors can have a fold change in expression (induction of expression) of 3 to 100 fold. The upper end of the range can vary depending on the copy number of the control region and/or biosensor, ratio of regulators, specifics of the construct, growth conditions for the cells, etc. The bisabolol responsive control regions and/or biosensors can have a coefficient of variation of 1-5%. The bisabolol responsive control regions and/or biosensors can have a coefficient of variation of about 2%.

### Reporters

The reporter can be selected from, for example, fluorescent reporters, bioluminescent reporters, and enzyme reporters. Other reporters are well known in the art and can be used as the reporter in the biosensors of the invention. For example, the biosensor could encode a polypeptide that activates the expression of another gene which provides a detectable reporter signal. The reporter can be selected because it will produce a detectable signal when expressed at the desired level from a bisabolol responsive control region. The reporter can be selected to provide a signal that distinguishes high transcription/expression rates from the bisabolol responsive control regions from medium and low expression rates for the bisabolol responsive control regions. The reporter can be selected to provide a signal that distinguishes medium transcription/expression rates from low expression rates for the bisabolol responsive control regions. The reporter can be selected to provide a signal that distinguishes between low level rates of expression for the bisabolol responsive control regions.

The fluorescent reporter can include, for example, green fluorescent protein from Aequorea victoria or Renilla reniformis, and active variants thereof (e.g., blue fluorescent protein, yellow fluorescent protein, cyan fluorescent protein, etc.); fluorescent proteins from Hydroid jellyfishes, Copepod, Ctenophora, Anthrozoas, and Entacmaea quadricolor, and active variants thereof; and phycobiliproteins and active variants thereof.

If the reporter is a bioluminescent reporter, any number of bioluminescent proteins can be used as the reporter. These include, for example, aequorin (and other Ca⁺² regulated photoproteins), luciferase based on luciferin substrate, luciferase based on Coelenterazine substrate (e.g., Renilla, Gaussia, and Metridina), and luciferase from Cypridina, and active variants thereof.

The reporter can be an enzyme including for example, acetylcholinesterase, alkaline phosphatase, chloramphenicol acetyltransferase, peroxidase, Gemini, or β-lactamase. Many other enzymes are well-known in the art and can be used as reporters for the biosensors of the invention.

Reactants paired with aceyticholinesterase include, for example, acetylthiocholine, or ThioStar® (which is commercially available from Arbor Assays or Kamiya Biomedical Co.) Reactants paired with alkaline phosphatase include, for example, p-aminophenyl phosphate (commercially available from Sigma Aldrich), PNPP (p-Nitrophenyl Phosphate, Disodium Salt), CSPD® chemiluminescent reactant, 1,2-dioxetane chemiluminescent reactant, DynaLight™ Substrate with RapidGlow™ Enhancer, which are all commercially available from ThermoFisher Scientific. Reactants paired with chloramphenicol acetyltransferase include, for example, FAST CAT® Green (deoxy), which is commercially available from ThermoFisher Scientific. Redox reactants paired with peroxidase, include, for example, hydroquinone, hydroxymethyl ferrocene, osmium complex, p-aminophenol, m-aminophenol, and o-aminophenol (o-AP). Other reactants for peroxidase include, for example, ABTS (2,2′-Azinobis [3-ethylbenzothiazoline-6-sulfonic acid]-diammonium salt), OPD (o-phenylenediamine dihydrochloride), TMB (3,3′,5,5′-tetramethylbenzidine), SuperSignal ELISA Pico Chemiluminescent Substrate, QuantaBlu NS/K Fluorogenic Substrate, QuantaRed Enhanced Chemifluorescent HRP Substrate (ADHP), Amplex Red reagent, all of which are commercially available from ThermoFisher Scientific. Reactants paired with β-lactamase, include, for example, C3′ thiolate-substituted cephalosporins. Other reactants for β-lactamase include, for example, CCF2-FA, CCF2-AM, CCF4-AM, Fluorocillin™ Green reagent, LyticBLAzer™ h-BODIPY® FL Substrate, which is commercially available from ThermoFisher Scientific.

The reporter can be detectable by multiple imaging modalities, for example tyrosinase which has been shown to yield photoacoustic imaging (PAI), MM and PET (with a suitable radiotracer) signals (see, e.g., Qin, C. et al., “Tyrosinase as a multifunctional reporter gene for photoacoustic/MRI/PET triple modality molecular imaging,” Scientific Rep. 3:1490 (2013), which is incorporated by reference in its entirety for all purposes). Alternatively, the reporter gene can be a fusion protein comprising two or more reporters linked together (e.g., a luciferase-GFP-thymidine kinase triple fusion reporter). (Ray P. et al., “Imaging tri-fusion multimodality reported gene expression in living subjects,” Cancer Res. 64:1323-1330 (2004), which is incorporated by reference in its entirety for all purposes).

### Selectable Markers

Selectable markers are well-known in the art for prokaryotic and eukaryotic cells, including host cells of the invention. Generally, the selection gene encodes a protein necessary for the survival or growth of transformed host cells grown in a selective culture medium. Host cells not transformed with and expressing the construct containing the selection gene will not survive in the culture medium. Typical selection genes encode proteins that (a) confer resistance to antibiotics or other toxins, or (b) complement auxotrophic deficiencies.

Selectable markers may confer resistance (or ability to grow) to a number of different antibiotics or toxins including, for example, ampicillin, erythromycin, chloramphenicol, kanamycin, methotrexate, neomycin, spectinomycin, or tetracycline. Other selectable markers suitable for use in the invention may be found at the Antibiotic resistance genes database at ardb.cbcb.umd.edu, which is incorporated by reference in its entirety for all purposes.

Selectable markers may also complement auxotrophic deficiencies including, for example, amino acid auxotrophies caused by the loss of an enzyme activity needed to make the amino acid (such auxotrophs can be complemented by a nucleic acid encoding an enzyme with an activity that can replace the lost activity). Other auxotrophies that may be used in selection scheme (with the selectable marker being a nucleic acid that complements the auxotrophy) include, for example, carbon utilization auxotrophs, nucleic acid auxotrophs, vitamin or cofactor auxotrophs, etc.

### Bisabolol Responsive Genes

The genes and gene products that can be made in response to bisabolol may originate from Pseudomonas and include, for example, a sensor domain-containing diguanylate cyclase (SEQ ID NO: 1), a PAS domain-containing protein (SEQ ID NO: 2 or 4), a CadC family transcriptional regulator (SEQ ID NO: 3), SEQ ID NO: 5, SEQ ID NO: 6, and a C4-dicarboxylate ABC transporter substrate-binding protein (SEQ ID NO: 7). The genes and gene products that can be made in response to bisabolol can be a sensor domain-containing diguanylate cyclase (SEQ ID NO: 1), and a PAS domain-containing protein (SEQ ID NO: 2). The gene and gene product made in response to bisabolol can be a sensor domain-containing diguanylate cyclase (SEQ ID NO: 1). The gene can encode and gene products can be polypeptides encoded by polynucleotides that hybridize under stringent hybridization conditions to a nucleic acid encoding one or more of SEQ ID NOs: 1-7. The gene can encode and gene products can be polypeptides encoded by polynucleotides that hybridize under stringent hybridization conditions to a nucleic acid encoding one or more of SEQ ID NOs: 1-2. The gene can encode and gene products can be polypeptides encoded by polynucleotides that hybridize under stringent hybridization conditions to a nucleic acid encoding one or more of SEQ ID NO: 1. The gene can encode and gene products can be polypeptides having 70%, 80%, 90%, 95% or 99% sequence identity with one of SEQ ID NOs: 1-7. The gene can encode and gene products can be polypeptides having 70%, 80%, 90%, 95% or 99% sequence identity with one of SEQ ID NOs: 1-2. The gene can encode and gene products can be polypeptides having 70%, 80%, 90%, 95% or 99% sequence identity with one of SEQ ID NO: 1.

The genes and gene products that can be made in response to bisabolol may also originate from Bradyrhizobium and include, for example, a serine hydrolase (SEQ ID NO: 8), SEQ ID NO: 9, SEQ ID NO: 10, a N-acetyltransferase (SEQ ID NO: 11), a glycoside-hydrolase-family-3-domain-protein-K05349-beta-glucosidase (SEQ ID NO: 12), a phosphoribosylaminoimidazole-succinocarboxamide synthase (SEQ ID NO: 13), a enoyl-CoA hydratase (SEQ ID NO: 14), a sulfate permease from the SulP family (SEQ ID NO: 15), a succinate semialdehyde dehydrogenase (SEQ ID NO: 16), SEQ ID NO: 17, a 1,4-butanediol diacrylate esterase (SEQ ID NO: 18), an AMP-binding protein or fatty-acyl-CoA synthase (SEQ ID NO: 19), SEQ ID NO: 20, an Arc family DNA binding domain-containing protein (SEQ ID NO: 21), a regulator of protease activity HflC from the stomatin/prohibitin superfamily (SEQ ID NO: 22), an adenosine or carbohydrate kinase (SEQ ID NO: 23), a signal transduction histidine kinase (SEQ ID NO: 24), a murein biosynthesis integral membrane protein MurJ (SEQ ID NO: 25), a tryptophanyl-tRNA synthetase (SEQ ID NO: 26), an universal stress protein UspA (SEQ ID NO: 27), an Fe—S cluster biogenesis protein NfuA, 4Fe-4S-binding domain (SEQ ID NO: 28), an adenosine(37)-N6)-threonylcarbamoyltransferase complex dimerization subunit type 1 TsaB (SEQ ID NO: 29), a ribosomal-protein-alanine N-acetyltransferase (SEQ ID NO: 30), a transcriptional repressor, ferric uptake regulator, Fur family (SEQ ID NO: 31), a N6-isopentenyl adenosine(37)-C2)-methylthiotransferase MiaB (SEQ ID NO: 32), a phosphate starvation-inducible protein PhoH (SEQ ID NO: 33), a rRNA maturation RNase YbeY (SEQ ID NO: 34), a CBS domain containing DNA-binding protein (SEQ ID NO: 35), and an apolipoprotein N-acyltransferase (SEQ ID NO: 36). The genes can encode and gene products made in response to bisabolol can be a 1,4-butanediol diacrylate esterase (SEQ ID NO: 18), an AMP-binding protein or fatty-acyl-CoA synthase (SEQ ID NO: 19), SEQ ID NO: 20, an Arc family DNA binding domain-containing protein (SEQ ID NO: 21), a regulator of protease activity HflC from the stomatin/prohibitin superfamily (SEQ ID NO: 22). The genes can encode and gene products made in response to bisabolol can be a 1,4-butanediol diacrylate esterase (SEQ ID NO: 18) and an AMP-binding protein or fatty-acyl-CoA synthase (SEQ ID NO: 19). The gene can encode and gene product made in response to bisabolol can be an AMP-binding protein or fatty-acyl-CoA synthase (SEQ ID NO: 19). The genes can encode and gene products made in response to bisabolol can be SEQ ID NO: 20, an Arc family DNA binding domain-containing protein (SEQ ID NO: 21), and a regulator of protease activity HflC from the stomatin/prohibitin superfamily (SEQ ID NO: 22). The gene can encode and gene product made in response to bisabolol can be SEQ ID NO: 20. The gene can encode and gene products can be polypeptides encoded by polynucleotides that hybridize under stringent hybridization conditions to a nucleic acid encoding one or more of SEQ ID NOs: 8-36. The gene can encode and gene products can be polypeptides encoded by polynucleotides that hybridize under stringent hybridization conditions to a nucleic acid encoding one or more of SEQ ID NOs: 18-22. The gene can encode and gene products can be polypeptides encoded by polynucleotides that hybridize under stringent hybridization conditions to a nucleic acid encoding one or more of SEQ ID NOs: 20-22. The gene can encode and gene products can be polypeptides encoded by polynucleotides that hybridize under stringent hybridization conditions to a nucleic acid encoding one or more of SEQ ID NO: 20. The gene can encode and gene products can be polypeptides having 70%, 80%, 90%, 95% or 99% sequence identity with one of SEQ ID NOs: 8-36. The gene can encode and gene products can be polypeptides having 70%, 80%, 90%, 95% or 99% sequence identity with one of SEQ ID NOs: 18-22. The gene can encode and gene products can be polypeptides having 70%, 80%, 90%, 95% or 99% sequence identity with one of SEQ ID NOs: 20-22. The gene can encode and gene products can be polypeptides having 70%, 80%, 90%, 95% or 99% sequence identity with one of SEQ ID NO: 20.

### Nucleic Acids

Nucleic acids can encode, at least in part, the nucleic acid constructs with a bisabolol biosensors. The nucleic acids can include nucleic acids encoding polypeptides naturally expressed from the bisabolol responsive control region. The nucleic acids may be natural, synthetic or a combination thereof.

The nucleic acids can include expression constructs, such as plasmids, or viral vectors, or linear vectors, or vectors that integrate into chromosomal DNA. Expression constructs can contain a nucleic acid sequence that enables the construct to replicate in one or more selected host cells (e.g., an origin of replication). Such sequences are well known for a variety of cells. E.g., the origin of replication from the plasmid pBR322 is suitable for most Gram-negative bacteria. In eukaryotic host cells, e.g., mammalian cells, the expression construct can be integrated into the host cell chromosome and then the construct replicates with the host chromosome. Similarly, constructs can be integrated into the chromosome of prokaryotic cells.

In general, expression constructs containing replication and control sequences that are derived from species compatible with the host cell are used in connection with a suitable host cell. The expression construct ordinarily carries a replication site, as well as marking sequences that are capable of providing phenotypic selection of the construct in transformed cells. For example, E. coli is typically transformed using pBR322, a plasmid derived from an E. coli species (see, e.g., Bolivar et al., (1977) Gene, 2: 95). pBR322 contains genes for ampicillin and tetracycline resistance and thus provides easy means for identifying transformed cells.

The constructs used can be stimulated to increase (or decrease) copy number in a suitable host cell. This copy control can be used to change the window of detection/selection for the bisabolol responsive biosensors that are cloned in the constructs. For example, the CopyControl Cloning System vectors which are sold by Epicentre can be used in the invention to make biosensor clones whose copy number can be inducibly changed (using arabinose). These copy number controllable constructs may be used in conjunction with the EPI300 E. coli strain which is also sold by Epicentre. The CopyControl Cloning System can be used to induce a high copy number for bisabolol responsive biosensors.

Expression constructs also generally contain a selection gene, also termed a selectable marker. Selectable markers are well-known in the art for prokaryotic and eukaryotic cells, including host cells of the invention. Generally, the selection gene encodes a protein necessary for the survival or growth of transformed host cells grown in a selective culture medium. Host cells not transformed with the construct containing the selection gene will not survive in the culture medium. Typical selection genes encode proteins that (a) confer resistance to antibiotics or other toxins, e.g., ampicillin, neomycin, methotrexate, spectinomycin, chloramphenicol, kanamycin, or tetracycline, (b) complement auxotrophic deficiencies, e.g., the gene encoding D-alanine racemase for Bacilli unable to make D-alanine because of a mutant D-alanine racemase. An exemplary selection scheme utilizes a drug to arrest growth of a host cell. Those cells that are successfully transformed with a heterologous gene produce a protein conferring drug resistance and thus survive the selection regimen. Other selectable markers for use in bacterial or eukaryotic (including mammalian) systems are well-known in the art.

Expression construct for producing polypeptides contain a suitable control region that is recognized by the host organism and is operably linked to the nucleic acid encoding the polypeptide of interest. Promoters used in the constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences can interact with proteins or other biomolecules to carry out (turn on/off, regulate, modulate, etc.) transcription. “Constitutive” promoters are those that drive expression continuously under most environmental conditions and states of development or cell differentiation. “Inducible” or “regulatable” promoters direct expression of the nucleic acid of the invention under the influence of environmental conditions or developmental conditions. Examples of environmental conditions that may affect transcription by inducible promoters include anaerobic conditions, elevated temperature, drought, or the presence of light.

Promoters suitable for use with prokaryotic hosts include the beta-lactamase and lactose promoter systems (Chang et al., (1978) Nature, 275: 615; Goeddel et al., (1979) Nature, 281: 544), the arabinose promoter system (Guzman et al., (1992) J. Bacteriol., 174: 7716-7728), alkaline phosphatase, a tryptophan (trp) promoter system (Goeddel, (1980) Nucleic Acids Res., 8: 4057 and EP 36,776) and hybrid promoters such as the tac promoter (deBoer et al., (1983) Proc. Natl. Acad. Sci. USA, 80: 21-25). Other exemplary bacterial promoters include lad, lacZ, T3, T7, gpt, lambda PR, and PL. Other bacterial promoters suitable for expression vectors are also well known in the art. Exemplary eukaryotic promoters include CMV immediate early, HSV thymidine kinase, early and late SV40, LTRs from retrovirus, and mouse metallothionein I. The nucleotide sequences of these and many other promoters have been published, thereby enabling a skilled worker to operably ligate them to DNA encoding the polypeptide of interest (Siebenlist et al, (1980) Cell, 20: 269) using linkers or adaptors to supply any required restriction sites. See also, e.g., Sambrook et al., Molecular Cloning: A Laboratory Manual, Cold Spring Harbor Laboratory Press, Cold Spring Harbor, N.Y. (1989); and Current Protocols in Molecular Biology, Ausubel et al., eds, Green Publishers Inc. and Wiley and Sons, N.Y (1994), both of which are incorporated by reference in their entirety for all purposes. The bisabolol responsive control regions are used in the expression construct to produce constructs that express a nucleic acid (e.g., encoding a heterologous polypeptide) in response to bisabolol.

Control regions for use in bacterial systems also generally contain a Shine-Dalgarno (S.D.) sequence operably linked to the DNA encoding the polypeptide of interest. The Shine-Dalgarno sequence and the initiating ATG codon are used in the initiation of translation by the ribosome in bacterial systems.

Expression constructs typically have promoter elements, e.g., enhancers, to regulate the frequency of transcriptional initiation. Typically, these are located in the region 30-110 base pairs upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 base pairs apart before activity begins to decline. Depending on the promoter, it appears that individual elements can function either cooperatively or independently to activate transcription.

The nucleic acid encoding a polypeptide can be easily prepared from an amino acid sequence of the polypeptide of interest using the genetic code. The nucleic acid encoding a polypeptide can be prepared using a standard molecular biological and/or chemical procedure. For example, based on the base sequence, a nucleic acid can be synthesized, and the nucleic acid of the present invention can be prepared by combining DNA fragments which are obtained from a cell or other nucleic acid using a polymerase chain reaction (PCR).

The nucleic acids encoding polypeptides can be linked to other nucleic acid so the a suitable promoter is operably linked to the nucleic acid encoding the polypeptide. The nucleic acids encoding a polypeptide can be also linked to, in order to attain efficient transcription of the nucleic acid, other regulatory elements that cooperate with a promoter or a transcription initiation site, for example, a nucleic acid comprising an enhancer sequence, or a terminator sequence. In addition, a gene that can be a marker for confirming expression of the nucleic acid (e.g. a drug resistance gene, a gene encoding a reporter enzyme, or a gene encoding a fluorescent protein) may be incorporated in the nucleic acid.

When the nucleic acid of the present invention is introduced into a host cell, the nucleic acid may be combined with a substance that promotes transference of the nucleic acid into a cell, for example, a reagent for introducing a nucleic acid such as a liposome or a cationic lipid, in addition to the aforementioned excipients.

A nucleic acid may encode the polypeptide of any one or more of SEQ ID NOs: 1-36. Nucleic acids may include those that hybridize under stringent hybridization conditions to a nucleic acid encoding one or more of SEQ ID NOs: 1-36. The nucleic acids can encode a polypeptide that has 70%, 80%, 90%, 95% or 99% sequence identity with one of SEQ ID NOs: 1-36. The nucleic acids can encode one of SEQ ID NOs: 1-36, hybridize under stringent hybridization conditions with a nucleic acid encoding one of SEQ ID NOs: 1-36, or encode a polypeptide that has 70%, 80%, 90%, 95% or 99% sequence identity with one of SEQ ID NOs: 1-36. A nucleic acid of the invention may encode the polypeptide of one of SEQ ID NOs: 1-2 or 18-22. Nucleic acids may include those that hybridize under stringent hybridization conditions to a nucleic acid encoding one or more of SEQ ID NOs: 1-2 or 18-22. The nucleic acids may encode a polypeptide that has 70%, 80%, 90%, 95% or 99% sequence identity with one of SEQ ID NOs: 1-2 or 18-22. The nucleic acids may encode a polypeptide that has 70%, 80%, 90%, 95% or 99% sequence identity with one of SEQ ID NOs: 1-2 or 20. The nucleic acids may encode one of SEQ ID NOs: 1-2 or 18-22, hybridize under stringent hybridization conditions with a nucleic acid encoding one of SEQ ID NOs: 1-2 or 18-22, or can encode a polypeptide that has 70%, 80%, 90%, 95% or 99% sequence identity with one of SEQ ID NOs: 1-2 or 18-22.

Polynucleotides may include polynucleotides that encode polypeptides having 70%, 80%, 90%, 95% or 99% sequence identity with one of SEQ ID NOs: 1-36. Polypeptides may include polypeptides encoded by nucleic acids that hybridize under stringent hybridization conditions with a nucleic acid encoding one of SEQ ID NOs: 1-36. The polypeptides can include one of SEQ ID NOs: 1-36, polypeptides encoded by nucleic acids that hybridize under stringent hybridization conditions with a nucleic acid encoding one of SEQ ID NOs: 1-36 or polypeptides that have 70%, 80%, 90%, 95% or 99% sequence identity with one of SEQ ID NOs: 1-36. Polynucleotides can include poly nucleotides that encode polypeptides having 70%, 80%, 90%, 95% or 99% sequence identity with one of SEQ ID NOs: 1-2 or 18-22. Polynucleotides can encode polypeptides having 70%, 80%, 90%, 95% or 99% sequence identity with one of SEQ ID NOs: 1-2 or 18-22. Polynucleotides can encode polypeptides having 70%, 80%, 90%, 95% or 99% sequence identity with one of SEQ ID NOs: 1 or 20. Polynucleotides can include polynucleotides that hybridize under stringent hybridization conditions with a polynucleotide encoding one of SEQ ID NOs: 1-2 or 18-22. The polynucleotides can hybridize under stringent hybridization conditions with a nucleic acid encoding one of SEQ ID NOs: 1-2 or 18-22. The polynucleotides can hybridize under stringent hybridization conditions with a nucleic acid encoding one of SEQ ID NOs: 1 or 20.

The bisabolol responsive control region includes SEQ ID NOs: 41-50, 136 from Pseudomonas, SEQ ID NOs: 62-135 from Bradyrhizobium, and/or SEQ ID NOs: 51-60. The bisabolol responsive control region can be found in SEQ ID NO: 38 and can include the promoters found at nucleotides 6202 to 6230, 6206 to 6234, 6215 to 6243, 6220 to 6248, or 6364-6393. The bisabolol responsive control region can be found in SEQ ID NO: 61 and includes the promoters found at nucleotides 724 to 752, 1865 to 1894, 1866 to 1894, 1867 to 1895, 1867 to 1897, 1867 to 1896, 2378 to 2408, 2489 to 2519, 2505 to 2533, 5485 to 5515, 5489 to 5518, 6744 to 6772, 7608 to 7636, 8858 to 8888, 9375 to 9403, 9375 to 9404, 9711 to 9740, 9794 to 9824, 9794 to 9822, 9795 to 9824, 9795 to 9823, 12854 to 12882, 13275 to 13305, 13276 to 13305, 13689 to 13719, 13694 to 13723, 13704 to 13733, 13704 to 13734, 14675 to 14704, 15024 to 15052, 15043 to 15073, 15045 to 15073, 16384 to 16414, 16386 to 16414, 16405 to 16435, 17181 to 17210, 17727, 1717757, 19865 to 19894, 19865 to 19895, 21013 to 21042, 21022 to 21050, 23046 to 23076, 23750 to 23778, 24341 to 24370, 24409 to 24438, 26357 to 26386, 29660 to 29688, or 29660 to 29690. The bisabolol responsive control region can include at least one of SEQ ID NOs: 41-47, 99-102, or 136. The bisabolol responsive control region can be SEQ ID NO: 136. The bisabolol responsive control region can be SEQ ID NO: 99. The bisabolol responsive control regions can include those nucleic acids that hybridize under stringent hybridization conditions with SEQ ID NOs: 41-47, 99-102, or 136.

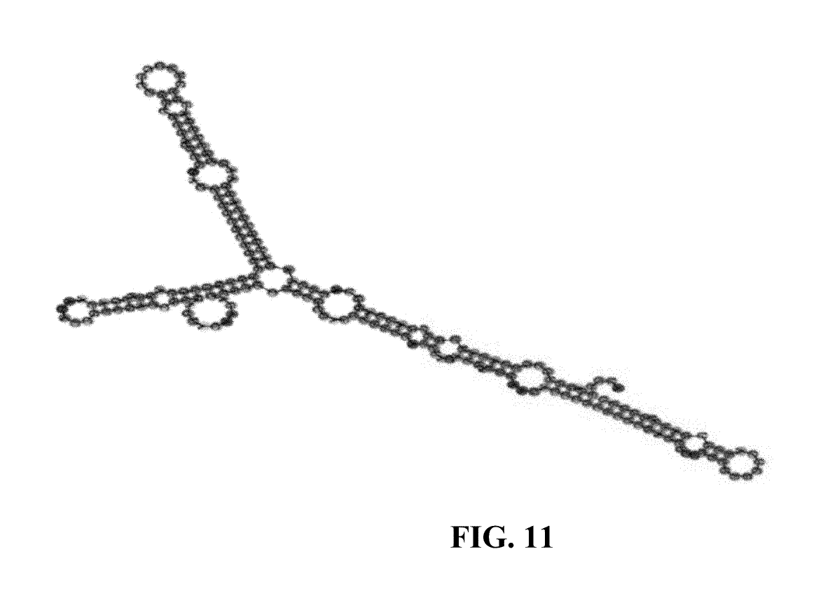

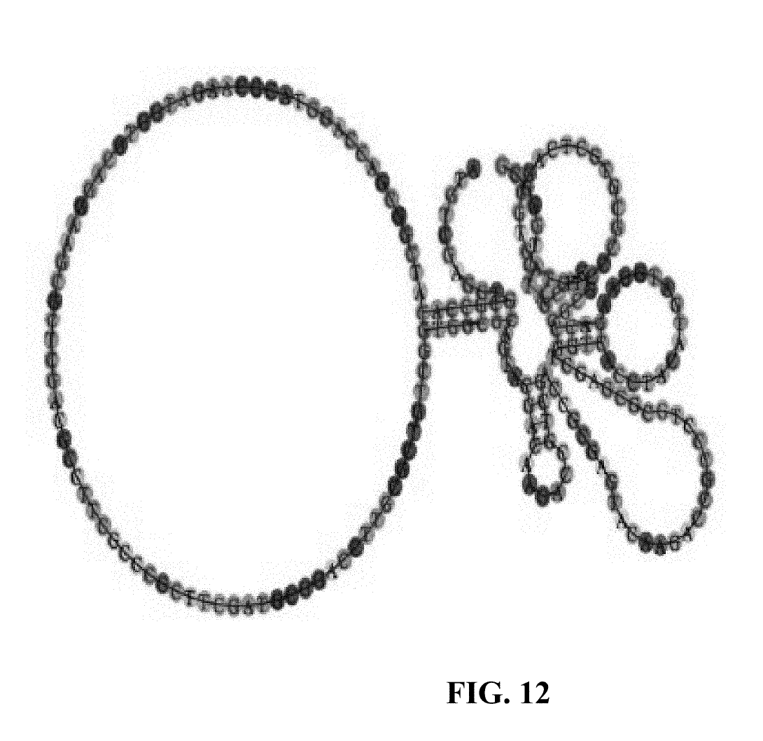

The bisabolol responsive control region can include control elements in the RNA transcript which are encoded in SEQ ID NO: 37. For example, FIG. 11 depicts a MFE structure and FIG. 12 depicts a centroid structure.

### Host Cells

Various host cells can be used with the polynucleotides and polypeptides described herein. The host cell may be any of the host cells familiar to those skilled in the art, including prokaryotic cells and eukaryotic cells, such as bacterial cells, fungal cells, yeast cells, mammalian cells, insect cells, or plant cells. Suitable prokaryotic host cells for expression of the biosensors and/or metagenomic libraries of the invention are well known in the art. Suitable prokaryote host cells include bacteria, e.g., eubacteria, such as Gram-negative or Gram-positive organisms, for example, any species of Acidovorax, Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas, including, e.g., E. coli, B. subtilis, P. aeruginosa, Salmonella typhimurium, Bacillus cereus, Pseudomonas fluorescens, Serratia marcescens, Clostridium acetobutylicum, Clostridium Beijerinckii, Clostridium saccharoperbutylacetonicum, Clostridium saccharobutylicum, Clostridium aurantibutyricum, or Clostridium tetanomorphum.

One example of an E. coli host is E. coli 294 (ATCC 31,446). Other strains such as EPI300 E. coli, E. coli B, E. coli X1776 (ATCC 31,537), and E. coli W3110 (ATCC 27,325) are also suitable. These examples are illustrative rather than limiting. Strain W3110 is a typical host because it is a common host strain for recombinant DNA product fermentations. In one aspect of the invention, the host cell should secrete minimal amounts of proteolytic enzymes. For example, strain W3110 may be modified to affect a genetic mutation in the genes encoding proteins, with examples of such hosts including E. coli W3110 strains 1A2, 27A7, 27B4, and 27C7 described in U.S. Pat. No. 5,410,026 issued Apr. 25, 1995, which is incorporated by reference in its entirety for all purposes.

The host cells can be plant cells. The plant cells can be cells of monocotyledonous or dicotyledonous plants, including, but not limited to, alfalfa, almonds, asparagus, avocado, banana, barley, bean, blackberry, brassicas, broccoli, cabbage, canola, carrot, cauliflower, celery, cherry, chicory, citrus, coffee, cotton, cucumber, eucalyptus, hemp, lettuce, lentil, maize, mango, melon, oat, papaya, pea, peanut, pineapple, plum, potato (including sweet potatoes), pumpkin, radish, rapeseed, raspberry, rice, rye, sorghum, soybean, spinach, strawberry, sugar beet, sugarcane, sunflower, tobacco, tomato, turnip, wheat, zucchini, and other fruiting vegetables (e.g. tomatoes, pepper, chili, eggplant, cucumber, squash etc.), other bulb vegetables (e.g., garlic, onion, leek etc.), other pome fruit (e.g. apples, pears etc.), other stone fruit (e.g., peach, nectarine, apricot, pears, plums etc.), Arabidopsis, woody plants such as coniferous and deciduous trees, an ornamental plant, a perennial grass, a forage crop, flowers, other vegetables, other fruits, other agricultural crops, herbs, grass, or perennial plant parts (e.g., bulbs; tubers; roots; crowns; stems; stolons; tillers; shoots; cuttings, including un-rooted cuttings, rooted cuttings, and callus cuttings or callus-generated plantlets; apical meristems etc.). The term “plants” refers to all physical parts of a plant, including seeds, seedlings, saplings, roots, tubers, stems, stalks, foliage and fruits.

The host cells can be algal and/or photosynthetic, including but not limited to algae or photosynthetic cells of the genera Agmenellum, Amphora, Anabaena, Ankistrodesmus, Botryococcus, Boekelovia, Borodinella, Botryococcus, Carteria, Chaetoceros, Chlamydomonas, Chlorella, Chlorococcum, Chlorogonium, Chrysosphaera, Cricosphaera, Cryptomonas, Cyclotella, Dunaliella, Ellipsoidon, Eremosphaera, Euglena, Fragilaria, Gleocapsa, Gloeothamnion, Hymenomonas, Isochrysis, Lepocinclis, Monoraphidium, Nannochloris, Nannochloropsis, Navicula, Nephrochloris, Nitschia, Nitzschia, Ochromonas, Oocystis, Oscillatoria, Nitzschia, Pascheria, Phagus, Phormidium, Platymonas, Pleurochrysis Prototheca, Pyrobotrys Scenedesmus, Spirogyra, Tetraedron, Tetraselmis, or Volvox. The host cell can be Botryococcus braunfi, Prototheca krugani, Prototheca moriformis, Prototheca portoricensis, Prototheca stagnora, Prototheca wickerhamii, or Prototheca zopfii.

The eukaryotic cells can be fungi cells, including, but not limited to, fungi of the genera Aspergillus, Candida, Chlamydomonas, Chrysosporium, Cryotococcus, Debaromyces, Fusarium, Hansenula, Kluyveromyces, Neotyphodium, Neurospora, Penicillium, Pichia, Saccharomyces, Schizosaccharomyce, Trichoderma, Xanthophyllomyces, Yarrowia, and Zygosaccharomyces. Exemplary fungi cells include Saccharomyces cerevisiae, Saccharomyces carlsbergensis, Saccharomyces lactis, Schizosaccharomyces pompe, Kluyveromyces lactis, Pichia pastoris, Hansenula polymorpha, or filamentous fungi, e.g. Trichoderma, Aspergillus sp., including Aspergillus niger, Aspergillus phoenicis, Aspergillus carbonarius.

Exemplary insect cells include any species of Spodoptera or Drosophila, including Drosophila S2 and Spodoptera Sf9. Exemplary animal cells include CHO, COS or Bowes melanoma or any appropriate mouse or human cell line known to person of skill in the art.

### Introduction of Polynucleotides to Host Cells

The nucleic acids can encode the bisabolol responsive biosensors and can be inserted into a construct(s) that is introduced into a plurality of cells. The constructs can encode a bisabolol responsive control region operably linked to a nucleic acid encoding a heterologous polypeptide. The constructs can encode a gene from which the bisabolol responsive control region can be derived. For example, the gene may be selected from SEQ ID NOs: 1-36.

The nucleic acid(s) described herein can be introduced to the eukaryotic cell by transfection (e.g., Gorman, et al. Proc. Natl. Acad. Sci. 79.22 (1982): 6777-6781, which is incorporated by reference in its entirety for all purposes), transduction (e.g., Cepko and Pear (2001) Current Protocols in Molecular Biology unit 9.9; DOI: 10.1002/0471142727.mb0909s36, which is incorporated by reference in its entirety for all purposes), calcium phosphate transformation (e.g., Kingston, Chen and Okayama (2001) Current Protocols in Molecular Biology Appendix 1C; DOI: 10.1002/0471142301.nsa01cs01, which is incorporated by reference in its entirety for all purposes), cell-penetrating peptides (e.g., Copolovici, Langel, Eriste, and Langel (2014) ACS Nano 2014 8 (3), 1972-1994; DOI: 10.1021/nn4057269, which is incorporated by reference in its entirety for all purposes), electroporation (e.g Potter (2001) Current Protocols in Molecular Biology unit 10.15; DOI: 10.1002/0471142735.im1015s03 and Kim et al (2014) Genome 1012-19. doi:10.1101/gr.171322.113, Kim et al. 2014 describe the Amaza Nucleofector, an optimized electroporation system, both of these references are incorporated by reference in their entirety for all purposes), microinjection (e.g., McNeil (2001) Current Protocols in Cell Biology unit 20.1; DOI: 10.1002/0471143030.cb2001s18, which is incorporated by reference in its entirety for all purposes), liposome or cell fusion (e.g., Hawley-Nelson and Ciccarone (2001) Current Protocols in Neuroscience Appendix 1F; DOI: 10.1002/0471142301.nsa01fs10, which is incorporated by reference in its entirety for all purposes), mechanical manipulation (e.g. Sharon et al. (2013) PNAS 2013 110(6); DOI: 10.1073/pnas.1218705110, which is incorporated by reference in its entirety for all purposes) or other well-known techniques for delivery of nucleic acids to host cells. Once introduced, the nucleic acids can be expressed episomally, or can be integrated into the genome of the host cell using well known techniques such as recombination (e.g., Lisby and Rothstein (2015) Cold Spring Harb Perspect Biol. Mar. 2; 7(3). pii: a016535. doi: 10.1101/cshperspect.a016535, which is incorporated by reference in its entirety for all purposes), non-homologous integration (e.g., Deyle and Russell (2009) Curr Opin Mol Ther. 2009 August; 11(4):442-7, which is incorporated by reference in its entirety for all purposes) or transposition (as described above for mobile genetic elements). The efficiency of homologous and non-homologous recombination can be facilitated by genome editing technologies that introduce targeted double-stranded breaks (DSB). Examples of DSB-generating technologies are CRISPR/Cas9, TALEN, Zinc-Finger Nuclease, or equivalent systems (e.g., Cong et al. Science 339.6121 (2013): 819-823, Li of al. Nucl. Acids Res (2011): gkr188, Gaj et al. Trends in Biotechnology 31.7 (2013): 397-405, all of which are incorporated by reference in their entirety for all purposes), transposons such as Sleeping Beauty (e.g., Singh et al (2014) Immunol Rev. 2014 January; 257(1):181-90. doi: 10.1111/imr.12137, which is incorporated by reference in its entirety for all purposes), targeted recombination using, for example, FLP recombinase (e.g., O'Gorman, Fox and Wahl Science (1991) 15:251(4999):1351-1355, which is incorporated by reference in its entirety for all purposes), CRE-LOX (e.g., Sauer and Henderson PNAS (1988): 85; 5166-5170), or equivalent systems, or other techniques known in the art for integrating the nucleic acids of the invention into the eukaryotic cell genome.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

Techniques for transforming a wide variety of higher plant species are well known and described in the technical and scientific literature. See, e.g., Weising (1988) Ann. Rev. Genet. 22:421-477; U.S. Pat. No. 5,750,870, which are both incorporated by reference in their entirety for all purposes.

### Methods for Using Bisabolol Responsive Biosensors

The biosensors can be used to identify when bisabolol is present in an environment. The biosensor can be in a host cell and the host cell can be placed in an environment. The host cell with the biosensor can provide a reporter signal when bisabolol is present. The bisabolol can be made by the host cell, by other cells (e.g., microorganisms) in the environment, or can be introduced into the environment from an exogenous (to the environment) source. The reporter signal from the biosensor can be used to quantify the amount of bisabolol in the environment. The reporter can provide real-time measurements of the bisabolol in the environment.

The biosensor and host cell can be used to screen for genes and/or gene products involved in bisabolol metabolism. The bisabolol gene and/or gene products can be involved in bisabolol synthesis, catabolism, regulation (e.g., regulation of expression, and/or allosteric and other regulation of enzymes in pathways) and/or energy metabolism. The biosensor and host cell can be used to identify clones that produce more bisabolol than other clones.

The copy number of the bisabolol responsive biosensor can be changed (e.g., increased) to change the response curve of the biosensor to bisabolol. For example, increasing the copy number of the biosensor increases the number of biosensors in the cell. The cells with the biosensor can be grown with high amounts of spectinomycin (e.g., 200-500 μg/ml) with the stimulus. The higher spectinomycin reduces the background level of reporter expression in the control samples and allows detection of reporter activity at lower amounts of stimulus. The higher spectinomycin and copy number can be used individually or together to change the response of the biosensor to bisabolol. The higher spectinomycin and/or copy number can be used to increase the sensitivity of detection with low amounts of bisabolol or high amounts of bisabolol. The higher spectinomycin and/or copy number can be used to increase the dynamic range of bisabolol detection by the biosensor.

The clones with functional biosensors can be used in a cell free protein synthesis format. Such cell free systems include, for example, those described in Chong, Curr. Protoc. Mol. Biol. 108:16.30.1-16.30.11 (2015); Swartz, Nat. Biotechnol. 27:731-732 (2009); Zemella et al., ChemBioChem 16:2420-2431 (2015), all of which are incorporated by reference in their entirety for all purposes. Such cell free systems can be used to test samples for bisabolol.

The bisabolol responsive biosensors can be used to screen a group of cells for cells that respond to bisabolol. The group of cells can be a community of microorganisms from an environment, or a selection of microorganisms from different environments. Nucleic acids encoding the bisabolol responsive biosensor can be placed into the cells in the group, the cells are grown under a desired set of conditions, and then the cells are screened or selected for expression by the bisabolol responsive biosensor. Screened or selected cells have expressed the reporter or selective marker of the biosensor above background and so are likely making bisabolol. The bisabolol responsive biosensor can be used to screen or select for bisabolol responsive clones from groups of cells having 10,000, 1,000, or 100 different types of cells in the group. Nucleic acids encoding the bisabolol responsive biosensors can be used to screen or select from 100,000,000, 1,000,000, 100,000, 10,000, 1,000, or 100 different types of cells per day. The cells with the bisabolol responsive biosensor can be screened individually or can be screened in groups of 10, 100, 1,000, 10,000, 100,000, 1,000,000, or 100,000,000.

### Methods for Using Bisabolol Responsive Control Regions and Genes

The control regions that can be responsive to bisabolol can be used as inducible expression systems for the recombinant expression of polypeptides in a host cell. These control regions can be responsive to bisabolol and can be used to express nucleic acids encoding polypeptides involved in bisabolol metabolism in the host cell.

The genes and/or gene products can be responsive to bisabolol and can be used to engineer host cells to have biochemical pathways that use and/or make bisabolol. These biochemical pathways may be anabolic pathways that make other products that use bisabolol as an intermediate. These biochemical pathways may also be catabolic pathways that used bisabolol as an intermediate.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1. Bisabolol Responsive Biosensors

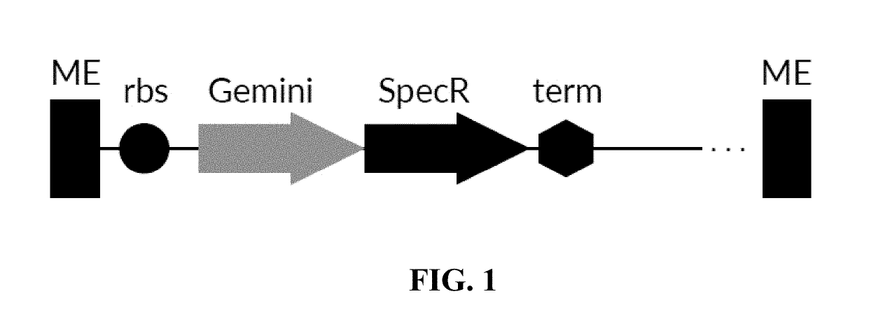

A metagenomic library made from environmental samples obtained from hots springs, permafrost, sea water, soil, and hydrocarbon resource samples (from soil) was made using the Meta-G-Nome™ DNA Isolation Kit and CopyControl™ Fosmid Library Production Kit obtained from Epicentre. 300-400 fosmid clones from this library were obtained and mixed with a Tn5 transposase and sufficient biosensor construct (FIG. 1) was added to give about 10 biosensor inserts per fosmid. The transposase randomly integrated the biosensor construct into the fosmid vector, and the modified fosmid library with biosensor constructs was transformed into E. coli strain EPI300™ (commercially sold by Epicentre). The E. coli EPI300™ transformed with the modified fosmid library was plated on LB media with 20 μg/ml spectinomycin and 0.01% arabinose. This selected for fosmid clones that had a basal or constitutive level of biosensor transcription sufficient to express enough Spec^{r} so the host cells could grow on media with 20 μg/ml spectinomycin.

About 20,000 fosmid clones with biosensors were found to have sufficient basal transcription in E. coli EPI300™ to provide spectinomycin resistance. The individual clones were picked and placed into 96 well plates with 20 μg/ml spectinomycin. Some of these clones were checked for basal expression of the Gemini reporter and no detectable fluorescence was found for the tested clones.

These functional biosensors were interrogated with (−)-α-bisabolol and clones that produced reporter fluorescence above control in response to the (−)-α-bisabolol were selected. Two bisabolol positive clones were selected and designated PB2 and PB3. PB2 was obtained from a fosmid with an insert originating from Pseudomonas, and PB3 was obtained from a fosmid with an insert originating from Bradyrhizobium.

The sequence of PB2 is found in SEQ ID NOs: 37-39 with the biosensor (SEQ ID NO: 39) oriented so that PB2 upstream (SEQ ID NO: 37) is located upstream of the coding sequence in the biosensor insert, and PB2 downstream (SEQ ID NO: 38) is located downstream of the coding sequence for the biosensor insert. The PB2 upstream sequence in SEQ ID NO: 37 is for the antisense strand (in relation to the biosensor insert, SEQ ID NO: 39) and so the complementary strand (sense strand in relation to the biosensor insert) is joined with the sense strand of the biosensor insert (SEQ ID NO: 39). Fosmid pCC1fos (SEQ ID NO: 40) contains the PB2 insert at the Eco721 site (CACGTG) of the Fosmid. The PB2 insert is in reverse orientation to SEQ ID NO: 40, so the biosensor coding strand (SEQ ID NO: 39) in PB2 is in the complementary strand relative to plasmid strand having the sequence of SEQ ID NO: 40.

The sequence of PB3 is found in SEQ ID NO: 61 and the biosensor (SEQ ID NO: 39) is inserted near base pair 14,600 of SEQ ID NO: 61. The biosensor insert is oriented in PB3 so that the coding strand (sense strand) for the biosensor insert is in the complementary strand relative to the PB3 sequence in SEQ ID NO: 61.

### Example 2. Response of Bisabolol Biosensors with High and Low Copy Number

As described above, the copy number of the fosmids with the PB2 biosensor and the PB3 biosensor can by increased in E. coli EPI300™ host cells that are treated with arabinose to induce an increase in copy number of the fosmid. E. coli EPI300™ with the PB2 or PB3 biosensors were treated (high copy number) or not treated (low copy number) with arabinose and then screened for reporter activity (Gemini reporter fluorescence from the GFP portion).

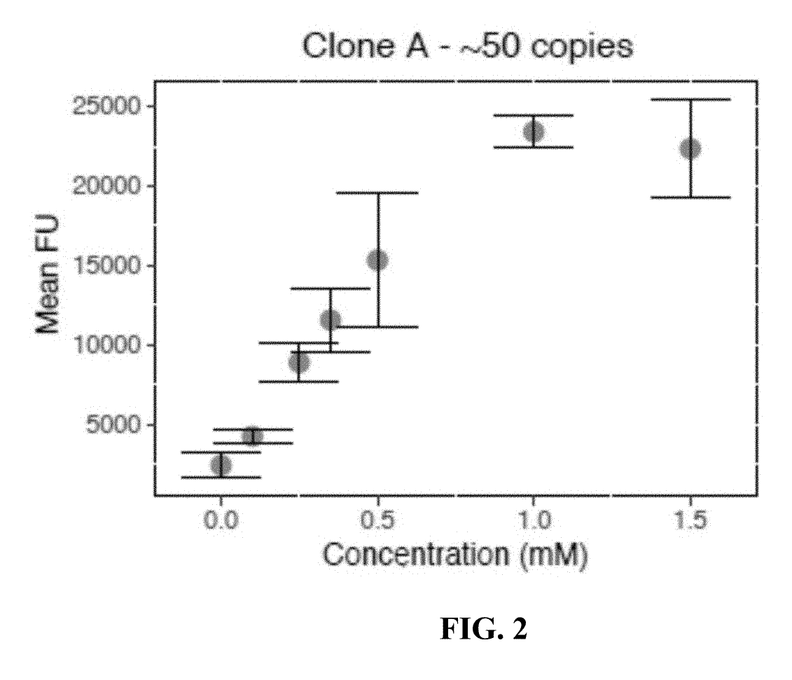

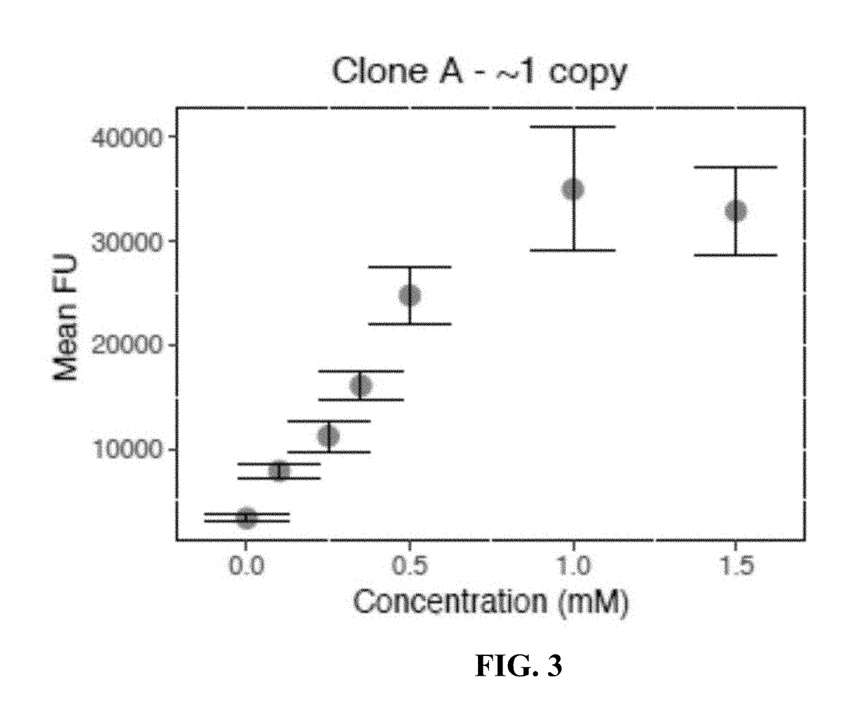

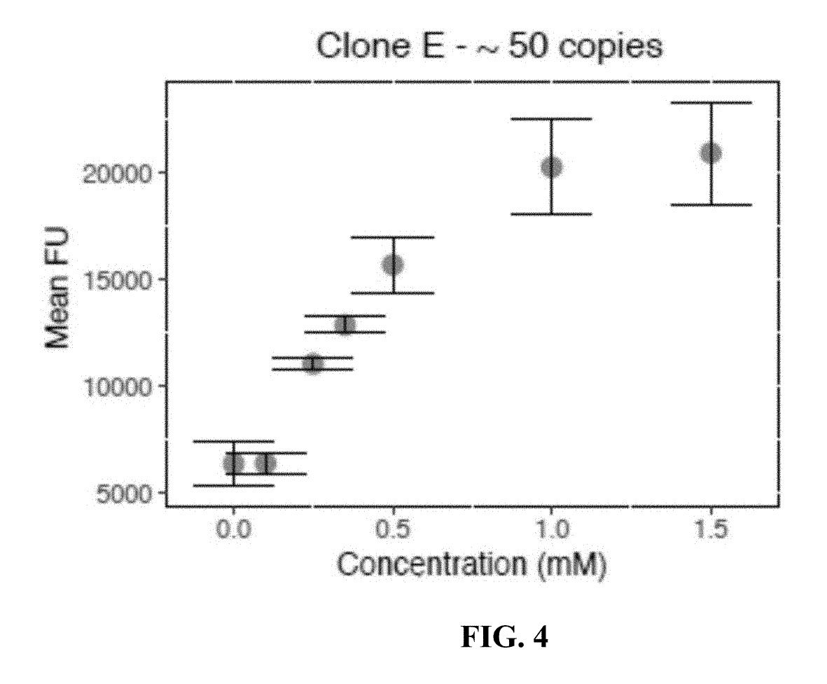

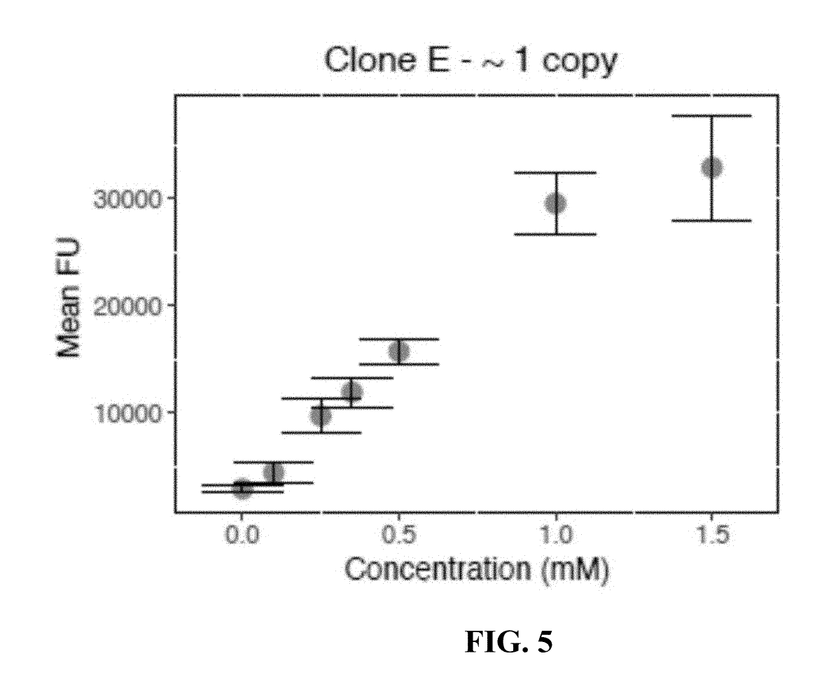

FIG. 2-5 show the fluorescence results for E. coli EPI300™ with high copy number PB2 or PB3, or low copy number PB2 or PB3. Both the PB2 and PB3 biosensors at both high and low copy number showed a dose response of fluorescence activity when the E. coli EPI300™ with PB2 or PB3 were treated with 0.0, 0.1, 0.25, 0.4, 0.5, 1.0 and 1.5 mM (−)-α-bisabolol. Both biosensors show a dose response between 0.05 and 1.0 mM at which point the fluorescence plateaus.

### Example 3. Response of Bisabolol Biosensors at Low Copy Number and with Antibiotic

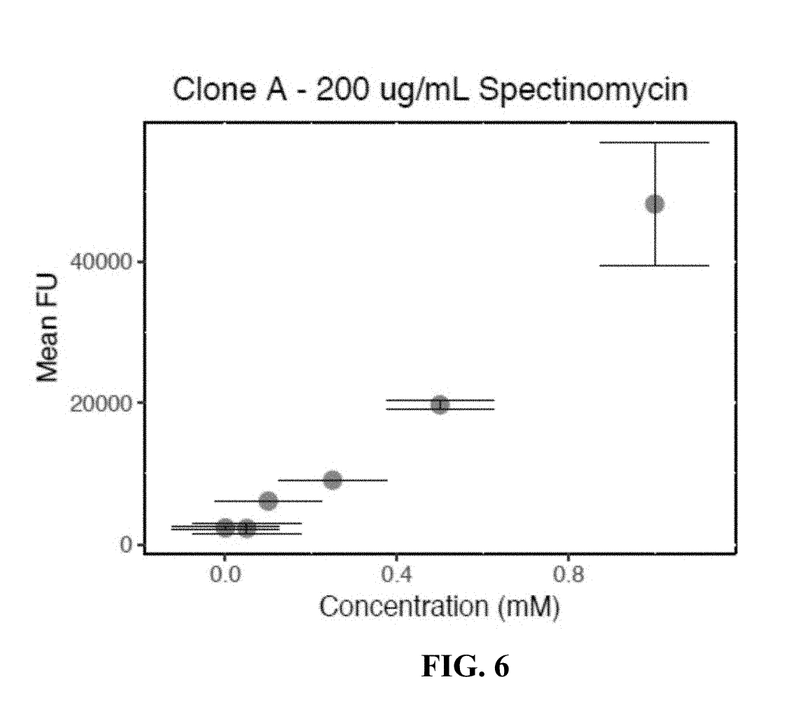

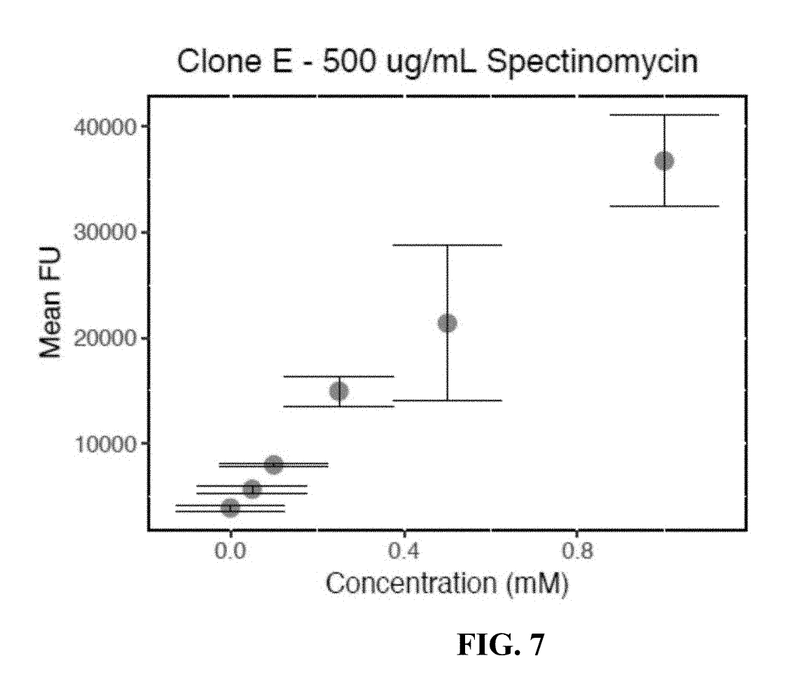

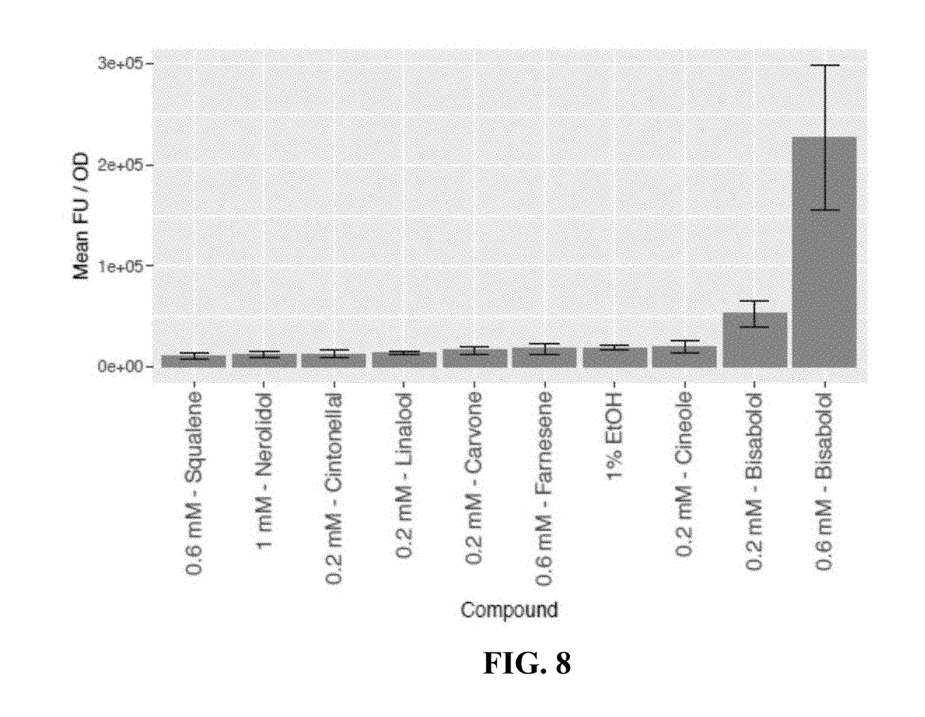

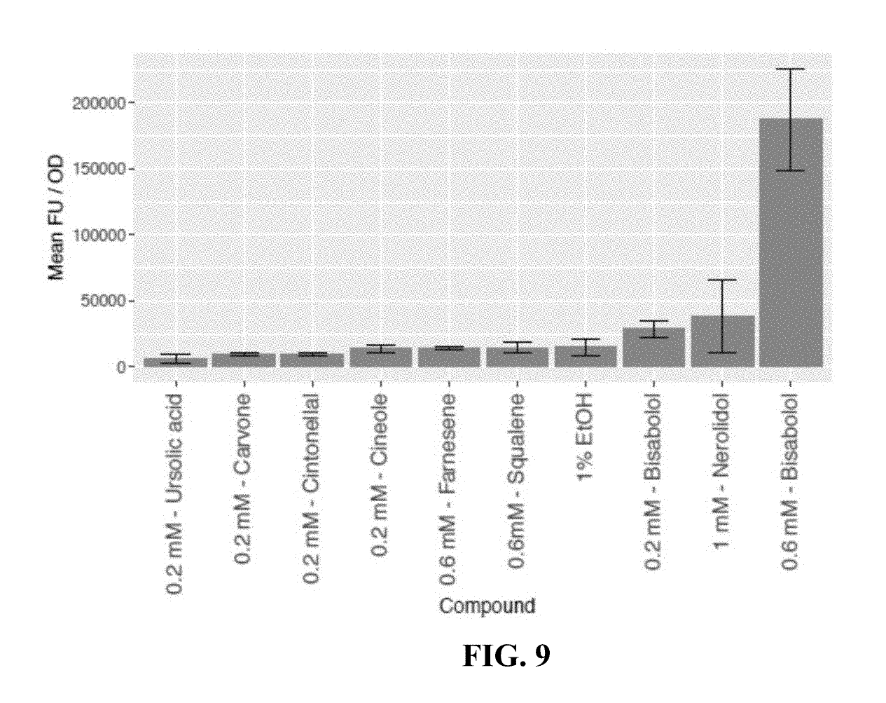

E. coli EPI300™ with fosmids containing PB2 or PB3 were grown without arabinose (low copy number) and in the presence of spectinomycin (200 μg/ml for PB2 and 500 μg/ml for PB3). The host cells were treated with different concentrations of (−)-α-bisabolol (0.0, 0.1, 0.25, 0.4, 0.5, 1.0 and 1.5 mM). FIGS. 6 and 7 show the fluorescence results for PB2 and PB3. Both biosensors show a dose response between 0.05 and 1.0 mM (−)-α-bisabolol.

### Example 4. Response of Biosensors to (−)-α-Bisabolol and Other Sesquiterpenes

E. coli EPI300™ with fosmids containing PB2 or PB3 were grown with 0.01% arabinose for high copy number of the biosensor, or grown in the absence of arabinose for low copy number of the biosensor, and then treated with a sesquiterpene: (−)-α-bisabolol, squalene, nerolidol, cintonellal, carvone, farnesene, or cineole. PB2 was also tested against linalool and PB3 was tested against ursolic acid.

E. coli EPI300™ with PB2 did not respond above background to squalene, nerolidol, cintonellal, linalool, carvone, farnesene, or cineole. E. coli EPI300™ with PB3 did not respond above background to ursolic acid, carvone, cintonellal, cineole, farnesene, and squalene. E. coli EPI300™ with PB3 did response to 1 mM nerolidol with comparable activity to PB3 treated with 0.2 mM (−)-α-bisabolol.

Among the compounds tested, PB2 shows high specificity for induction by (−)-α-bisabolol. In comparison, PB3 shows stimulation by (−)-α-bisabolol and nerolidol.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. Citation of references herein is not an admission that such references are prior art to an embodiment of the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

We claim:

1. A method for monitoring (−)-α-bisabolol, comprising the steps of: providing a host cell, wherein the host cell comprises a polynucleotide, wherein the polynucleotide comprises a nucleic acid encoding a reporter, a nucleic acid encoding a selection marker, a nucleic acid encoding a first ribosome binding site, a nucleic acid encoding a second ribosome binding site, and a control region that increases expression in response to a (−)-α-bisabolol wherein the control region includes at least one of a SEQ ID NOs: 41-60, 62-136, wherein the nucleic acid encoding the first ribosome binding site is operably linked to the nucleic acid encoding the reporter, wherein the nucleic acid encoding the second ribosome binding site is operably linked to the nucleic acid encoding the selection marker, and wherein the nucleic acid encoding the reporter and the nucleic acid encoding the selection marker are expressed from the control region that increases expression in response to the (−)-α-bisabolol; expressing the nucleic acid encoding the reporter when a (−)-α-bisabolol is present; and detecting the reporter.

2. The method of claim 1, wherein the host cell is a bacterium.

3. The method of claim 2, wherein the bacterium is an Escherichia coli.

4. The method of claim 28, wherein the (−)-α-bisabolol is present in a media.

5. The method of claim 1, wherein the (−)-α-bisabolol is present in the host cell.

6. The method of claim 5, wherein the (−)-α-bisabolol is made in the host cell.

7. The method of claim 5, wherein the (−)-α-bisabolol is made by other cells.

8. The method of claim 1, wherein the (−)-α-bisabolol is added to the media.

9. The method of claim 1, wherein the (−)-α-bisabolol is present in a complex mixture.

10. The method of claim 9, wherein the complex mixture is a sample or an extract from a complex source.

11. The method of claim 10, wherein the complex source is a food, an aqueous environment, a soil, a plant, or a gas.

12. The method of claim 1, further comprising the steps of adding a selective agent to the host cell and selecting for the host cell that expresses the selection marker.

13. The method of claim 1, wherein the selection marker is selected from the group consisting of an antibiotic resistance gene, a polypeptide that complements an auxotrophic deficiency, and a polypeptide that makes a critical nutrient.

14. The method of claim 13, wherein the selection marker is an antibiotic resistance gene.

15. The method of claim 14, wherein the antibiotic resistance is a spectinomycin resistance.

16. The method of claim 1, wherein the reporter is selected from the group consisting of an optical reporter, a fluorescent reporter, a bioluminescent reporter, a fusion protein reporter, an enzyme, and combinations of the foregoing.

17. The method of claim 16, wherein the reporter is an optical reporter.

18. The method of claim 17, wherein the reporter is a Gemini reporter.

19. The method of claim 1, wherein the control region includes at least one of SEQ ID NO: 41-47, 90-93, 99-102, 131 or 136.

20. The method of claim 19, wherein the control region includes one of a SEQ ID NO: 99, a SEQ ID NO: 131, or a SEQ ID NO: 136.
